SEQUENCE LISTING (11 sequences)

SEQ ID NO: 1 - protein, 193 aa, Mus musculus
MARARQEGSSPEPVEGLARDSPRPFPLGRLMPSAVSCSLCEPGLPAAPAAPALLPAAYLCAPTAPPAVTAALGGPRWPGGHRSRPRGPRPDGPQPSLSPAQQHLESPVPSAPEALAGGPTQAAPGVRVEEEEWAREIGAQLRRMADDLNAQYERRRQEEQHRHRPSPWRVMYNLFMGLLPLPRDPGAPEMEPN

SEQ ID NO: 2 - DNA, 720 bp, synthetic construct
  source 1..720: mol_type = other DNA
atggtgtctaagggcgaggaattgtttacaggtgtggtgcccatcctggtggagcttgatggcgatgtaaatggacacaaattctccgttagtggggaaggcgaaggggatgccacctacggtaagcttacgctgaaattcatctgcaccactggtaaactccccgtgccatggccaaccctggtcacgacccttacttatggggtgcagtgtttttcaaggtaccccgaccatatgaaacaacatgatttcttcaagtccgccatgccggaggggtacgtccaggagagaacaatctttttcaaagatgacgggaactacaagactcgcgcagaagtcaagtttgagggagacactctcgtaaaccgaattgaactgaaaggaattgactttaaggaagacggtaatatactgggccacaagctggagtataattataacagccataatgtgtatatcatggcagacaagcaaaagaacggcattaaagtgaacttcaagatccgtcacaatatcgaggatggcagcgtccagctggctgaccactaccagcagaacacacctattggagacggcccagttttactacctgacaaccactatctcagtacacagagcgccctctctaaggaccctaatgaaaagcgggatcatatggttttactggagtttgtcacagctgcgggaataaccttgggcatggatgagttgtacaaatga

SEQ ID NO: 3 - DNA, 1653 bp, synthetic construct
  source 1..1653: mol_type = other DNA
atggaagacgccaagaacattaagaagggccctgcacccttctacccactggaagacggtactgcaggggagcagctgcacaaggccatgaagcggtatgccctcgttcctggcactatcgccttcacagatgcccacatcgaagtagatatcacctatgctgagtactttgagatgagtgtgagactggcagaggcaatgaaacgttatggactgaacaccaaccatagaatcgtagtgtgctctgagaacagcttgcagttcttcatgcctgtcttaggagcactgttcataggcgtcgccgtggcaccagccaatgacatttacaatgagagggagctcctgaatagtatgggcataagccagccaacagtggttttcgtttccaagaaagggcttcaaaaaatcctgaatgtgcaaaagaagctccctatcatccagaagatcattataatggactcaaagactgattaccagggcttccagtccatgtatacatttgtcaccagccaccttccaccaggcttcaatgaatatgatttcgtgcctgagtcatttgacagggacaaaacaattgcactcattatgaattcttccgggtcaacaggtttgcccaagggggtggcgctaccacacagaacggcgtgtgtgcgctttagccatgctcgagaccccatcttcgggaatcagatcattcccgacactgccatcttgtctgtcgtcccttttcaccatggtttcggtatgttcaccacgttgggctacctgatctgtggtttccgggtagtactgatgtacaggtttgaagaagagctcttcctgcggagcctacaggactacaagatccagagcgcactgctggtgcccacccttttttcgttctttgccaaatccaccctgattgataaatatgacctatccaaccttcatgagatagcatctggaggtgctcctctgagtaaagaagtcggagaagctgtagccaagaggttccacctgccaggcattcgccaaggatatggcctgacagagactacaagtgccattttaataactccagagggagatgacaagcctggggctgtgggcaaagttgttccgttcttcgaagctaaggtggttgacctggacacaggaaaaaccctgggcgtcaaccagcgtggggaactctgcgtccgagggcccatgatcatgtctggctacgtgaacaaccccgaggccaccaatgccctcattgacaaggatggctggctccattcaggagacattgcctactgggacgaggatgaacacttttttattgtggacaggctcaagtcgcttatcaagtacaaaggctaccaggtggctcctgctgaattggaatccatcttacttcagcaccccaacatatttgatgcgggtgtggccggtctaccggatgatgatgcaggagagctgcccgctgctgttgttgtgctggagcatggtaagaccatgactgagaaggagattgtggactatgtagcgtctcaagtcacgaccgctaaaaaactaagagggggtgtggtctttgtggatgaggtcccaaaaggattgactgggaagctggatgctcgcaaaataagagaaatcctcatcaaagcaaagaagggagggaaaattgctgtctga

SEQ ID NO: 4 - DNA, 1032 bp, synthetic construct
  source 1..1032: mol_type = other DNA
atgtctaatctcctcactgtgcatcagaatcttccagctttaccggtagacgccacgtctgatgaagtgcgcaaaaatctcatggacatgttcagggaccggcaagccttcagtgagcacacatggaagatgttgttgtctgtgtgtcgctcctgggctgcctggtgcaaacttaacaacaggaagtggttccctgcagagcctgaggacgtcagagactatctgctctacttgcaagcacgaggactcgcggtaaagaccatccagcagcacctgggccagctgaacatgctgcacaggaggtctgggctgccccgaccaagtgactcaaatgctgtgtctctggtcatgagacgcatccgcaaggagaatgtggatgccggagaacgagccaagcaggctctggcttttgaacggacagactttgatcaggtgagatccctgatggagaactcagatagatgccaggacatccggaacctggcctttcttgggatagcctacaacaccttgctgaggatagcagaaattgccagaattcgggtcaaggacattagcaggacagatgggggcaggatgctcatccacattggccggactaaaacccttgtttcaactgcaggcgtggaaaaagccttgagcttaggtgtcaccaagctggtggagagatggatcagcgtctccggagttgcagacgacccaaataattatctcttctgtcgtgttcggaagaacggagttgcagcgccctcggctaccagccaactaagcacgagagctctggagggcatttttgaggccactcatcgcctgatctatggagcaaaagatgactccgggcagagatacctggcatggagtggtcatagtgctcgtgtcggtgctgcaagagatatggcccgggctggggtttccatacctgaaatcatgcaggctggtggctggacaaacgtgaacattgtgatgaactacatcaggaatctagattctgagacaggagccatggtgcgattactggaagatggcgattga

SEQ ID NO: 5 - RNA, 582 nt, synthetic construct
  source 1..582: mol_type = other RNA
atggccagagcaaggcaagaaggcagcagtccagaacctgtggaaggattggccagggactccccgagaccatttcctctcggccggctcatgcccagtgctgtcagctgctcattgtgtgagccaggccttccagcagcaccagctgcacccgccctgttgccagcagcttacctgtgcgcccctactgccccccctgccgtgaccgcggcacttggtggaccccgctggcctggtgggcacagaagcagaccgcgaggccccagaccagatggcccccagccttccctgtctcccgcgcagcagcacctggagtcccctgtaccatcagccccggaagctctggcagggggtcctacacaagccgctcctggggttcgggtggaggaagaagagtgggcccgagagatcggggctcagctgcgtaggatggctgatgacctcaatgctcagtatgaacgccggcgtcaggaggagcagcataggcatcgcccctctccctggagggtcatgtacaacctattcatgggattactgcctctccctcgggacccaggagccccagagatggagcccaactga

SEQ ID NO: 6 - RNA, 630 nt, synthetic construct
  source 1..630: mol_type = other RNA
atggcctccggccagggccccggcccccccaaggtgggctgcgacgagtccccctccccctccgagcagcaggtggcccaggacaccgaggaggtgttccgctcctacgtgttctacctgcaccagcaggagcaggagacccagggcgccgccgcccccgccaaccccgagatggacaacctgcccctggagcccaactccatcctgggccaggtgggccgccagctggccctgatcggcgacgacatcaaccgccgctacgacaccgagttccagaacctgctggagcagctgcagcccaccgccggcaacgcctacgagctgttcaccaagatcgcctcctccctgttcaagtccggcatctcctggggccgcgtggtggccctgctgggcttcggctaccgcctggccctgtacgtgtaccagcgcggcctgaccggcttcctgggccaggtgacctgcttcctggccgacatcatcctgcaccactacatcgcccgctggatcgcccagcgcggcggctgggtggccgccctgaacttccgccgcgaccccatcctgaccgtgatggtgatcttcggcgtggtgctgctgggccagttcgtggtgcaccgcttcttccgctcctag

SEQ ID NO: 7 - RNA, 834 nt, synthetic construct
  source 1..834: mol_type = other RNA
atggagaacaacaagacctccgtggactccaagtccatcaacaacttcgaggtgaagaccatccacggctccaagtccgtggactccggcatctacctggactcctcctacaagatggactaccccgagatgggcatctgcatcatcatcaacaacaagaacttccacaagtccaccggcatgtcctcccgctccggcaccgacgtggacgccgccaacctgcgcgagaccttcatgggcctgaagtaccaggtgcgcaacaagaacgacctgacccgcgaggacatcctggagctgatggactccgtgtccaaggaggaccactccaagcggtcctccttcgtgtgcgtgatcctgtcccacggcgacgagggcgtgatctacggcaccaacggccccgtggagctgaagaagctgacctccttcttccgcggcgactactgccgctccctgaccggcaagcccaagctgttcatcatccaggcctgccgcggcaccgagctggactgcggcatcgagaccgactccggcaccgacgaggagatggcctgccagaagatccccgtggaggccgacttcctgtacgcctactccaccgcccccggctactactcctggcgcaactccaaggacggctcctggttcatccagtccctgtgctccatgctgaagctgtacgcccacaagctggagttcatgcacatcctgacccgcgtgaaccgcaaggtggccaccgagttcgagtccttctccctggactccaccttccacgccaagaagcagatcccctgcatcgtgtccatgctgaccaaggagctgtacttctaccactag

SEQ ID NO: 8 - RNA, 912 nt, synthetic construct
  source 1..912: mol_type = other RNA
atgaccgacgaccaggactgcgccgccgagctggagaaggtggactcctcctccgaggacggcgtggacgccaagcccgaccgctcctccatcatctcctccatcctgctgaagaagaagcgcaacgcctccgccggccccgtgcgcaccggccgcgaccgcgtgcccacctacctgtaccgcatggacttccagaagatgggcaagtgcatcatcatcaacaacaagaacttcgacaaggccaccggcatggacgtgcgcaacggcaccgacaaggacgccggcgccctgttcaagtgcttccagaacctgggcttcgaggtgaccgtgcacaacgactgctcctgcgccaagatgcaggacctgctgcgcaaggcctccgaggaggaccactccaactccgcctgcttcgcctgcgtgctgctgtcccacggcgaggaggacctgatctacggcaaggacggcgtgacccccatcaaggacctgaccgcccacttccgcggcgaccgctgcaagaccctgctggagaagcccaagctgttcttcatccaggcctgccgcggcaccgagctggacgacggcatccaggccgactccggccccatcaacgacatcgacgccaacccccgcaacaagatccccgtggaggccgacttcctgttcgcctactccaccgtgcccggctactactcctggcgcaaccccggcaagggctcctggttcgtgcaggccctgtgctccatcctgaacgagcacggcaaggacctggagatcatgcagatcctgacccgcgtgaacgaccgcgtggcccgccacttcgagtcccagtccgacgacccccgcttcaacgagaagaagcagatcccctgcatggtgtccatgctgaccaaggagctgtacttctcccgctag

SEQ ID NO: 9 - RNA, 185 nt, synthetic construct
  source 1..185: mol_type = other RNA
tgataatagcaaacaccattgtcacactccagctggagcctcggtggccatgcttcttgccccttgggcccaaacaccattgtcacactccatccccccagcccctcctccccttcctgcacccgtaccccccaaacaccattgtcacactccagtggtctttgaataaagtctgagtgggcggc

SEQ ID NO: 10 - DNA, 4818 bp, synthetic construct
  source 1..4818: mol_type = other DNA
atgaaacggacagccgacggaagcgagttcgagtcaccaaagaagaagcggaaagtctctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaactccggaggatctagcggaggctcctctggctctgagacacctggcacaagcgagagcgcaacacctgaaagcagcgggggcagcagcggggggtcagacaagaagtacagcatcggcctgaccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatggtgaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcattatcccccaccagatccacctgggagagctgcacgccattctgcggcggcagggcgatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggctgagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggcggccacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagggcaacagcgataagctgatcgccagaaagaaggactgggaccctaagaagtacggcggcttcaacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgtgctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcaaccggaagcaatacaacacgaccaaagaggtgctggacgccaccctgatccgtcagagcatcaccggcctgtacgagacacggatcgacctgtctcagctgggaggtgactctggcggctcaaaaagaaccgccgacggcagcgaattcgagcccaagaagaagaggaaagtctaa

SEQ ID NO: 11 - RNA, 20 nt, synthetic construct
  source 1..20: mol_type = other RNA
ttctccacaggagtcaggtg

(19) United States
(12) Patent Application Publication
(10) Pub. No.: US 2026/0191929 A1
(43) Pub. Date: Jul. 9, 2026

(54) HSC targeted LNP for delivery of pro-apoptotic mRNA and methods of use thereof

(71) Applicants: The Trustees of the University of Pennsylvania, Philadelphia, PA (US); The Children's Hospital of Philadelphia, Philadelphia, PA (US)

(72) Inventors: Hamideh Parhiz, Gladwyne, PA (US); Tyler Papp, Philadelphia, PA (US); Stefano Rivella, Philadelphia, PA (US); Laura Breda, Philadelphia, PA (US); Michael Triebwasser, Philadelphia, PA (US)

(21) Appl. No.: 19/136,642

(22) PCT Filed: Dec. 8, 2023
(86) PCT No.: PCT/US2023/083139

§ 371 Date: Jun. 6, 2025

Related U.S. Application Data
(60) Provisional application No. 63/386,754, filed on Dec. 9, 2022.

(51) Int. Cl. A61K 38/17; A61K 9/51; A61K 45/06; A61K 47/68; A61K 47/69; A61P 7/00

(52) U.S. Cl. CPC A61K 38/1709; A61K 9/5123; A61K 45/06; A61K 47/6849; A61K 47/6929; A61P 7/00

(57) ABSTRACT

The present invention relates to CD117 targeted LNP molecules for delivery of pro-apoptotic mRNA to hematopoietic stem cells (HSCs) and methods of use thereof for preconditioning a subject for hematopoietic stem cell therapy or to deplete the subject's HSC population for the treatment of a disease or disorder.

### CROSS REFERENCE TO RELATED APPLICATIONS

This application claims priority to U.S. Provisional Application No. 63/386,754, filed Dec. 9, 2022, which is hereby incorporated by reference herein in its entirety.

### BACKGROUND OF THE INVENTION

Allogeneic human stem cell transplantation (AlloHSCT) is the only cure for a diverse set of genetic disorders of the bone marrow, from immunodeficiency, bone marrow failure, to sickle cell disease (SCD). In the US, approximately 800 AlloHSCT procedures were done for non-malignant conditions in 2017. The success of AlloHSCT is limited by high mortality (3-10%) and morbidity due to myeloablative treatment and immune system incompatibility between the donor and recipient. This is important because less than 70% of SCD patients have an immunologically matched donor in the USA. Of those with a match, <25% will have a matched sibling donor, which carries the lowest risk of graft versus host disease complications (Mentzer et al., 1994, Am J Pediatr Hematol Oncol, 16 (1): p. 27-9).

For patients without a compatible donor, gene therapy (GT) is an emerging curative option. In GT the genetic defect in one's own HSC is corrected ex vivo and gene modified HSC are returned as an autologous HSCT (AutoHSCT) after conditioning.

Both Allo- and Auto-HSCT require the use of genotoxic chemotherapy drugs, such as Busulfan, to deplete existing HSC from the niche and make space for healthy donor cells or patients' corrected cells. This is a blunt tool that damages the DNA and cells throughout the body. In the peri-transplant period, mucositis, which causes pain and main necessitate parental nutrition, and organ toxicity can lead to acute liver, kidney or pulmonary failure. The spectrum of long-term consequences from conditioning includes growth impairment, endocrine deficit and infertility, multiple organ toxicity (such as cardiac and pulmonary disease), secondary malignant neoplasms and an increased risk of early death (Leiper, 2002, Br J Haematol, 118 (1): p. 3-22; Leiper, 2002, Br J Haematol, 118 (1): p. 23-43; Cohen et al., 2008, Bone Marrow Transplant, 2008. 41 Suppl 2: p. S43-8; Chemaitilly et al., 2010, Endocr Relat Cancer, 17 (3): p. R141-59). Given the severity of these morbidities, a less toxic method of depleting HSC from the bone marrow is urgently needed and, if successful, such a method would have invaluable health benefits for patients undergoing HSCT.

Efforts have been made to reduce the chemotherapy-related morbidity and mortality for HSCT, such as choosing less toxic chemotherapy or lower doses. Reduced-intensity conditioning, which uses lower doses of chemotherapy, is an option in conditions like immunodeficiencies or SCD where there does not need to be 100% replacement by the donor (Oved et al., 2019, Biol Blood Marrow Transplant, 25(3): p. 549-555). However, for the purpose of GT, most studies have only shown benefit when ≥80% HSC are replaced by gene modified HSC, thus necessitating fully myeloablative conditioning (Mansilla-Soto et al., 2016, Hum Gene Ther, 27(4): p. 295-304). Most recently, experimental approaches to reduce the “off target” toxicity associated with chemotherapy have focused on the use of antibodies against HSC markers. One includes targeting HSC using anti-CD45 and anti-CD117 (stem cell factor receptor, cKit) antibodies conjugated to a toxic drug (Palchaudhuri et al., 2016, Nat Biotechnol, 34(7): p. 738-45; Czechowicz et al., 2019, Nat Commun, 10(1): p. 617), but this approach has incomplete engraftment in a GT model and resulting hepatotoxicity (Gao et al., 2019, Blood Adv, 3(18): p. 2700-2711).

Hematopoietic stem cells (HSCs) divide throughout life allowing them to give rise to the blood and immune system due to their self-renewal ability. Their multipotency enables the formation of myeloid, including erythroid, megakaryocytic, and myeloid immune as well as lymphoid progenitors. HSCs are dependent on stromal-derived factors, including stem cell factor (SCF), which binds to the receptor c-Kit (CD117). CD117 is expressed on both short- and long-term HSCs and some hematopoietic progenitors. CD117 is internalized after binding of SCF, which may facilitate or augment LNP internalization. Replacement of diseased HSCs with healthy donor allogeneic HSCs can cure non-malignant hematopoietic disorders (NMHD), such as hemoglobinopathies and immunodeficiencies. Gene therapy for NMHD disorders is currently done ex vivo with lentiviral transduction of HSC or electroporation of purified genome editing reagents, but still requires HSCT with gene-modified, autologous HSC. Conditioning regimens, which include high-dose chemotherapy or radiation, are required for HSCs depletion to prepare a patient for BM transplant (BMT). These procedures carry significant acute and chronic systemic toxicities, including sterility and secondary malignancies due to accumulated DNA damage. Some NMHD, such as radiosensitive severe combined immunodeficiency or Fanconi anemia, are due to DNA repair pathway mutations. These patients experience excessive acute toxicity with alkylating chemotherapy or radiation as well as long-term increased rates of malignancy. Therefore, a novel methodology was developed to address two major challenges: modify HSC in vivo and establish a non-genotoxic alternative method.

Thus, there is a need in the art for improved targeted therapeutics for the treatment of diseases and disorders. The present invention addresses this need.

### SUMMARY OF THE INVENTION

In one embodiment, the invention relates to a composition for targeted delivery of an apoptosis-inducing agent to a target hematopoietic stem cell (HSC), the composition comprising an agent and a delivery vehicle, wherein the delivery vehicle comprises a CD117 targeting moiety specific for binding to the HSC, and wherein the agent comprises an mRNA molecule encoding an apoptosis inducing protein. In one embodiment, the apoptosis inducing protein is p53 upregulated modulator of apoptosis (PUMA), BCL2 Antagonist/Killer 1 (Bak-1), Caspase 3 (Cas-3) or Caspase 9 (Cas-9), or a functional variant or fragment thereof. In one embodiment, the mRNA molecule comprises SEQ ID NO:5, SEQ ID NO:6, SEQ ID NO:7 or SEQ ID NO:8 or a functional variant or fragment thereof.

In one embodiment, the mRNA molecule is an isolated nucleoside-modified mRNA molecule.

In one embodiment, the mRNA molecule comprises a binding site for miR-122.

In one embodiment, the miR-122 binding site is in the 3′UTR of the mRNA molecule.

In one embodiment, the isolated nucleoside-modified RNA comprises at least one selected from the group consisting of pseudouridine and 1-methylpseudouridine.

In one embodiment, the delivery vehicle comprises a lipid nanoparticle (LNP).

In one embodiment, the mRNA is encapsulated within the LNP.

In one embodiment, the invention relates to a method of treating a disease or disorder in a subject in need thereof, the method comprising administering a composition for targeted delivery of an apoptosis-inducing agent to a target hematopoietic stem cell (HSC), the composition comprising an agent and a delivery vehicle, wherein the delivery vehicle comprises a CD117 targeting moiety specific for binding to the HSC, and wherein the agent comprises an mRNA molecule encoding an apoptosis inducing protein to the subject. In one embodiment, the apoptosis inducing protein is p53 upregulated modulator of apoptosis (PUMA), BCL2 Antagonist/Killer 1 (Bak-1), Caspase 3 (Cas-3) or Caspase 9 (Cas-9), or a functional variant or fragment thereof. In one embodiment, the mRNA molecule comprises SEQ ID NO:5, SEQ ID NO: 6, SEQ ID NO:7 or SEQ ID NO:8 or a functional variant or fragment thereof.

In one embodiment, the disease or disorder is a blood monogenic disorder, genetic defect (e.g., beta thalassemia or sickle cell disease), bone marrow genetic defect, cancer, platelet disorder, red cell disorder, immunodeficiency, metabolic disease or an autoimmune disease.

In one embodiment, the composition is administered by intravenous (IV), intraosseous infusion (IO), intraperitoneal (IP), intradermal, subcutaneous, inhalation, intranasal, or intramuscular delivery.

In one embodiment, the invention relates to a method of preconditioning a subject for hematopoietic stem cell therapy (HSCT), the method comprising administering a composition for targeted delivery of an apoptosis-inducing agent to a target hematopoietic stem cell (HSC), the composition comprising an agent and a delivery vehicle, wherein the delivery vehicle comprises a CD117 targeting moiety specific for binding to the HSC, and wherein the agent comprises an mRNA molecule encoding an apoptosis inducing protein to the subject. In one embodiment, the apoptosis inducing protein is p53 upregulated modulator of apoptosis (PUMA), BCL2 Antagonist/Killer 1 (Bak-1), Caspase 3 (Cas-3) or Caspase 9 (Cas-9), or a functional variant or fragment thereof. In one embodiment, the mRNA molecule comprises SEQ ID NO: 5, SEQ ID NO:6, SEQ ID NO:7 or SEQ ID NO:8 or a functional variant or fragment thereof.

In one embodiment, the HSCT is autologous HSCT, including genetically modified autologous HSC transplantation (i.e., gene therapy).

In one embodiment, the composition is administered by intravenous (IV), intraosseous infusion (IO), intraperitoneal (IP), intradermal, subcutaneous, inhalation, intranasal, or intramuscular delivery.

In one embodiment, the method further comprises administering at least one of an antihistamine agent and an anti-inflammatory agent in combination with the composition comprising an agent and a delivery vehicle, wherein the delivery vehicle comprises a CD117 targeting moiety specific for binding to the HSC, and wherein the agent comprises an mRNA molecule encoding a pro-apoptotic protein (e.g., PUMA, Bak-1, Cas-3 or Cas-9). In one embodiment, the method comprises administering an antihistamine agent and an anti-inflammatory agent in combination with the composition comprising an agent and a delivery vehicle, wherein the delivery vehicle comprises a CD117 targeting moiety specific for binding to the HSC, and wherein the agent comprises an mRNA molecule encoding a pro-apoptotic protein (e.g., PUMA, Bak-1, Cas-3 or Cas-9). In one embodiment, the antihistamine agent is administered 1 week to 1 minute prior to administration of the composition comprising an agent and a delivery vehicle, wherein the delivery vehicle comprises a CD117 targeting moiety specific for binding to the HSC, and wherein the agent comprises an mRNA molecule encoding a pro-apoptotic protein (e.g., PUMA, Bak-1, Cas-3 or Cas-9), and the anti-inflammatory agent is administered 1 minute to 1 week following administration of the composition comprising an agent and a delivery vehicle, wherein the delivery vehicle comprises a CD117 targeting moiety specific for binding to the HSC, and wherein the agent in comprises an mRNA molecule encoding a pro-apoptotic protein (e.g., PUMA, Bak-1, Cas-3 or Cas-9). In one embodiment, the antihistamine agent is administered 1 day to 1 minute prior to administration of the composition comprising an agent and a delivery vehicle, wherein the delivery vehicle comprises a CD117 targeting moiety specific for binding to the HSC, and wherein the agent comprises an mRNA molecule encoding a pro-apoptotic protein (e.g., PUMA, Bak-1, Cas-3 or Cas-9), and the anti-inflammatory agent is administered 1 minute to 1 day following administration of the composition comprising an agent and a delivery vehicle, wherein the delivery vehicle comprises a CD117 targeting moiety specific for binding to the HSC, and wherein the agent comprises an mRNA molecule encoding a pro-apoptotic protein (e.g., PUMA, Bak-1, Cas-3 or Cas-9). In one embodiment, the antihistamine agent is administered 1 hour to 1 minute prior to administration of the composition comprising an agent and a delivery vehicle, wherein the delivery vehicle comprises a CD117 targeting moiety specific for binding to the HSC, and wherein the agent comprises an mRNA molecule encoding a pro-apoptotic protein (e.g., PUMA, Bak-1, Cas-3 or Cas-9), and the anti-inflammatory agent is administered 1 minute to 1 hour following administration of the composition comprising an agent and a delivery vehicle, wherein the delivery vehicle comprises a CD117 targeting moiety specific for binding to the HSC, and wherein the agent comprises an mRNA molecule encoding a pro-apoptotic protein (e.g., PUMA, Bak-1, Cas-3 or Cas-9).

In one embodiment, the invention relates to a method of depleting HSCs in a subject in need thereof, the method comprising administering a composition for targeted delivery of an apoptosis-inducing agent to a target hematopoietic stem cell (HSC), the composition comprising an agent and a delivery vehicle, wherein the delivery vehicle comprises a CD117 targeting moiety specific for binding to the HSC, and wherein the agent comprises an mRNA molecule encoding an apoptosis inducing protein to the subject. In one embodiment, the apoptosis inducing protein is p53 upregulated modulator of apoptosis (PUMA), BCL2 Antagonist/Killer 1 (Bak-1), Caspase 3 (Cas-3) or Caspase 9 (Cas-9), or a functional variant or fragment thereof. In one embodiment, the mRNA molecule comprises SEQ ID NO:5, SEQ ID NO:6, SEQ ID NO:7 or SEQ ID NO: 8 or a functional variant or fragment thereof.

In one embodiment, the disease or disorder is a blood monogenic disorder, genetic defect (e.g., beta thalassemia or sickle cell disease), bone marrow genetic defect, cancer, platelet disorder, red cell disorder, immunodeficiency, metabolic disease or an autoimmune disease.

In one embodiment, the composition is administered by intravenous (IV), intraosseous infusion (IO), intraperitoneal (IP), intradermal, subcutaneous, inhalation, intranasal, or intramuscular delivery.

In one embodiment, the method further comprises administering at least one of an antihistamine agent and an anti-inflammatory agent in combination with the composition comprising an agent and a delivery vehicle, wherein the delivery vehicle comprises a CD117 targeting moiety specific for binding to the HSC, and wherein the agent comprises an mRNA molecule encoding a pro-apoptotic protein (e.g., PUMA, Bak-1, Cas-3 or Cas-9). In one embodiment, the method comprises administering an antihistamine agent and an anti-inflammatory agent in combination with the composition comprising an agent and a delivery vehicle, wherein the delivery vehicle comprises a CD117 targeting moiety specific for binding to the HSC, and wherein the agent comprises an mRNA molecule encoding a pro-apoptotic protein (e.g., PUMA, Bak-1, Cas-3 or Cas-9). In one embodiment, the antihistamine agent is administered 1 hour to 1 minute prior to administration of the composition comprising an agent and a delivery vehicle, wherein the delivery vehicle comprises a CD117 targeting moiety specific for binding to the HSC, and wherein the agent comprises an mRNA molecule encoding a pro-apoptotic protein (e.g., PUMA, Bak-1, Cas-3 or Cas-9), and the anti-inflammatory agent is administered 1 minute to 1 hour following administration of the composition comprising an agent and a delivery vehicle, wherein the delivery vehicle comprises a CD117 targeting moiety specific for binding to the HSC, and wherein the agent comprises an mRNA molecule encoding a pro-apoptotic protein (e.g., PUMA, Bak-1, Cas-3 or Cas-9).

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1A through FIG. 1G depict representative data demonstrating in vitro targeting of whole bone marrow or hematopoietic progenitors (lin-) cells incubated with LNPs encapsulating luciferase (CD117/LNP-Luc) or Cre recombinase (CD117/LNP-Cre) mRNAs. FIG. 1A depicts the luciferase activity normalized by total protein in whole bone marrow cells incubated with varying doses (indicated in figure) of targeted or control LNP-luc for 18 hours in vitro (N=3). FIG. 1B depicts LNP-Luc treatment of Lineage negative (Lin−) bone marrow cells (N=3). FIGS. 1C-1G depict an assessment of ZsGreen+ reporter induction after CD117/LNP-Cre treatment in Ai6 cells triggered by removal of loxP flanked stop cassette by Cre. Treatment dose in bone marrow cells (FIG. 1C, FIG. 1E, FIG. 1G) or Lin-cells (FIG. 1D, FIG. 1F) and subsequent culture intervals stated in figure. All data represent mean±SD of 3 biological replicas.

FIG. 2A through FIG. 2D depict exemplary experimental data demonstrating delivery to antigen positive cells in whole bone marrow and viability in LNP-Cre treated bone marrow cells. FIG. 2A depicts the luciferase activity normalized to protein in cell lysate (cell number surrogate) and the frequency of antigen positive cells in whole bone marrow (WBM) cells (anti-CD45 [85%], anti-CD117 [2.8%], or unnormalized for control IgG/LNP. FIG. 2B depicts the viability of Ai6 WBM cells after 6, (FIG. 2C) 18 and (FIG. 2D) 18 hour+72 hours culture in vitro exposure to increasing doses of LNP (up to 1 μg) and assessed by AOPI staining.

FIG. 3A through FIG. 3H depict exemplary experimental data demonstrating CD117/LNP-Cre treatment ex vivo leads to near-complete tdTomato gene editing upon transplantation. (FIG. 3A and FIG. 3B) Percentage tdTomato marking in myeloid (Gr1+) (A) and lymphoid (CD3+, left, and B220+, right) (FIG. 3B) cells measured at 16 weeks after HSCT in lethally irradiated congenic CD45.1 recipients who received Ai14 BM treated ex vivo with 0.1 and 1 mg of control IgG/LNP-Cre or CD117/LNPCre. In (FIG. 3A) and (FIG. 3B), data represent mean±SEM of n=4 (for IgG/LNP-Cre at 1 mg only) or n=5 experimental animals per cohort. P values are from Tukey's multiple comparison test after one-way ANOVA. In (FIG. 3A), ****P<0.0001. In (FIG. 3B), *P<0.05, ****P<0.0001. (FIG. 3C) Kinetic analysis of erythroid editing measured up to 16 weeks after HSCT. Data represent mean±SD of n=4 or 5 experimental animals per cohort. (FIG. 3D) tdTomato marking in the BM and BM subsets: c-Kit+ (Lin-c-Kit+), LSK, and LT-HSCs (SLAM). Data represent mean±SEM of n=4 or 5 experimental animals per cohort [same animals as in (FIG. 3A) and (FIG. 3B)]. P values are Tukey's multiple comparison test after one-way ANOVA. ***P<0.001, ****P<0.0001. (FIG. 3E) CFU assay from Ai14 BM treated ex vivo with 0.1 mg or 1 mg of control IgG/LNP-Cre or CD117/LNP-Cre formulations or untreated. (FIG. 3F and FIG. 3G) Semiquantitative PCR of (FIG. 3F) BM and

(FIG. 3G) spleen genomic DNA isolated from the groups in (FIG. 3A) to (FIG. 3C) at 4 months after BMT. **271 base pair (bp) Cre-recombinase-edited genomic DNA (gDNA) region and *1142 bp unedited region are indicated.

FIG. 4A through FIG. 4F depict data demonstrating that ex vivo CD117-LNP/Cre treated HSC retain multi-lineage engraftment. (FIG. 4A) Peripheral blood donor chimerism in lethally irradiated congenic (CD45.1) recipients of ex vivo treated of bone marrow cells from Ai14 donor mice (CD45.2) with CD117/LNP-Cre (left) or Control IgG/LNP-Cre (right) across a 100-fold range in dose. (FIG. 4B) tdTomato+ cell frequency in red blood cells (RBC), white blood cells (CD45.2+), and granulocytes (Gr1+) cells from 8-16 weeks post-transplant after ex vivo treatment of Ai14 bone marrow cells with (FIG. 4B) CD117/LNP-Cre or (FIG. 4C) control IgG/LNP-Cre. (FIG. 4D) tdTomato+ cell frequency in peripheral blood myeloid (Gr1+) and lymphoid cells (CD3+ [T-cells], B220+ [B-cells]) and in bone marrow (BM) subsets (c-Kit, Lin-c-Kit subset, LSK, Lin-c-Kit Scal 1, SLAM, LSK CD150+CD48−) at 4 months after 0.01 or 0.05 mg of CD117/LNP-Cre or control IgG/LNP-Cre. For the groups in A-D, N=4 to 6, except untreated Ai14 (N=3). Mean+/−SEM are shown in A-D. P-values are Tukey's multiple comparison test after one-way ANOVA *p<0.05, **p<0.01 ***p<0.001, ****p<0.0001 (FIG. 4E) Colony forming unit assay from bone marrow at 4 months after transplantation with ex vivo treated Ai14 BM at the doses shown. (FIG. 4F) Percentage of total CFU that are tdTomato+16 weeks post-transplant with ex vivo treated BM. Quantification of images in E. N=4 independent samples for each group. Shown are the mean+/−SEM. P-value by paired t-test ****p<. 0001.

FIG. 5A through FIG. 5E depict exemplary data demonstrating that ex vivo CD117/LNP-Cre edited HSC persist upon secondary transplantation. tdTomato+ cell frequency in peripheral blood (FIG. 5A) red blood cells, (FIG. 5B) myeloid cells (Gr1+), and (FIG. 5C) lymphoid cells (CD3+ [T-cells], B220+ [B-cells]) 4 months after transplantation with bone marrow from primary chimeras of ex vivo CD117 or control IgG/LNP-Cre (0.01 μg mRNA) treated bone marrow (1 mouse from each in vivo treated cohort was used to engraft 7 secondary recipient mice). (FIG. 5D) Donor chimerism in peripheral blood at 4 months in secondary transplants. (FIG. 5E) Frequency of gene edited cells in whole bone marrow (BM) and bone marrow subsets (c-Kit, Lin-c-Kit+subset, LSK, Lin-c-Kit+Scal−, SLAM, LSK CD150+CD48−) in secondary chimeras at 16 weeks posttransplant. N=7 for each group in all panels. Mean+/−range are shown. P-value by Mann-Whitney test. ***p<0.001.

FIG. 6A through FIG. 6L depict exemplary data demonstrating CD117/LNP-Cre formulations lead to >50% tdTomato marking in LT-HSCs after in vivo injection. (FIG. 6A) Biodistribution of intravenous injection of 1 mg of targeted LNP-mRNA expression in vivo by means of luminescence imaging at 24 hours. A representative sample set of dissected mouse organs were analyzed 5 min after the administration of D-luciferin. (FIG. 6B to FIG. 6D) tdTomato+ cell frequency in peripheral blood (FIG. 6B) myeloid (Gr1+) and (FIG. 6C) lymphoid cells [CD3+ (T cells), B220+ (B cells)] and in (FIG. 6D) BM subsets (c-Kit, LSK, SLAM/LT-HSCs) at 4 months after in vivo treatment with 5 mg of CD117/LNP-Cre or control IgG/LNP-Cre. In (FIG. 6B), (FIG. 6C), and (FIG. 6D), data represent mean±SEM of n=5 experimental animals per cohort. P values are reported from paired t test. **P<0.01, ***P<0.001, ****P<0.0001. (FIG. 6E to FIG. 6G) tdTomato+ cell frequency in peripheral blood (FIG. 6E) myeloid, (FIG. 6F) lymphoid cells, and (FIG. 6G) BM subsets at 4 months after in vivo treatment with 5 or 1 mg of CD117/LNP-Cre. In (FIG. 6E), (FIG. 6F), and (FIG. 6G), data represent mean±SEM of n=7 (1 mg) and n=5 (5 mg) experimental animals per cohort. P values are reported from t test. ***P<0.001, ****P<0.0001. (FIG. 6H and FIG. 6I) Edited RBC frequency over time in Ai9 mice treated in vivo with (FIG. 6H) 5 mg of CD117/LNP-Cre or control IgG/LNP-Cre or with (FIG. 6I) 1 or 5 mg of CD117/LNP-Cre. In (H), data represent mean±SD of n=experimental animal per cohort. P values are reported from paired t test. ****P<0.0001. In (FIG. 6I), data represent mean±SD of n=7 (1 mg) and 5 (5 mg) experimental animals per cohort. P values are reported from t test. ****P<0.0001. (FIG. 6J) CFU assay from BM at 4 months after in vivo treatment with 5 mg control IgG/LNP-Cre (top), no treatment (middle), or 5 mg CD117/LNP-Cre (bottom). (FIG. 6K and FIG. 6L) Semiquantitative PCR of (FIG. 6K) BM and (FIG. 6L) spleen genomic DNA isolated from the animals in (FIG. 6B) to (FIG. 6D) at 4 months after BMT. **271 bp Cre-recombinase-edited gDNA region and *1142 bp unedited region are indicated.

FIG. 7A through FIG. 7H depict data demonstrating the CD117/LNP-Cre mediated in vivo editing of the dormant EPCR+HSC population and colony forming cells. (FIG. 7A) Gating scheme used to quantitate live whole bone marrow, cKit+Lin−, LSK, and SLAM cells and (FIG. 7B) relative frequency of EPCR+ cells within each subpopulation. (FIG. 7C) Representative contour plots of the EPCR+subpopulation within the SLAM population (left) and relative ZsGreen expression in the EPCR+ population (right) one month after in vivo treatment with control IgG or CD117/LNP-Cre (FIG. 7D). (FIG. 7E) Percentages of in vivo edited LT-HSC SLAM and LT-HSC EPCR+SLAM in Control IgG or CD117/LNP-Cre in Ai6 mice. N=5 experimental mice in each group. Mean+/−range shown. P-values are Tukey's multiple comparison test after one-way ANOVA ***p<0.0001. (FIG. 7F) Absolute number of CFU and (FIG. 7G) ZsGreen+CFU of BM from primary chimeras (N=3 per group) generated from in vivo treated Ai6 donors. Results are the average of three technical replicates for each animal (nine total CFU per group). (FIG. 7H) Percentage of ZsGreen+CFU in each cohort. p-value by t-test. *p-value<0.05, **p-value<0.01.

FIG. 8A through FIG. 8E depict data demonstrating the CD117/LNP-Cre edited HSC persist upon primary transplantation of BM from in vivo treated donors. tdTomato+ cell frequency in peripheral blood (FIG. 8A) red blood cells, (FIG. 8B) myeloid cells (Gr1+), and (FIG. 8C) lymphoid cells (CD3+ [T-cells], B220+[B-cells]) 4 months after transplantation with bone marrow from Ai9 mice injected in vivo with control IgG/LNP-Cre or CD117/LNPCre formulations (1 mouse from each in vivo treated cohort was used to engraft 7 primary recipient mice). (FIG. 8D) Donor chimerism in the primary chimeras at 16 weeks posttransplant. (FIG. 8E) Frequency of gene edited cells in whole bone marrow (BM) and bone marrow subsets (c-Kit, Lin-c-Kit+subset, LSK, Lin-c-Kit_{|}Scal_{|}, SLAM, LSK CD150_{|} CD48−) in primary chimeras at 16 weeks posttransplant. N=7 experimental mice per group for A-E. Shown is mean+/−SEM. p-value calculated t-test. *p<0.05, **p<0.01 ***p<0.001.

FIG. 9A and FIG. 9B depict data demonstrating the absolute cell counts of BM subpopulation in ex vivo and in vivo treated animals are consistent among cohorts. (FIG. 9A) Absolute cell number in BM after red blood cell lysis (upper left), cKit+Lin− (upper right), LSK (lower left) and SLAM (lower right) cells quantified by flow cytometry, upon exclusion of dead (7AAD+) cells. Samples obtained upon crushing one femur and leg from each mouse in ex vivo and (FIG. 9B) in vivo treated mice. N=5 to 7 animals per group. Mean+/−standard deviation shown. P-values are Tukey's multiple comparison test after one-way ANOVA *p<0.05 **p<0.01.

FIG. 10A through FIG. 10E depict data demonstrating the in vivo editing after CD117/LNP treatment. (FIG. 10A) Percentage of total CFU that are tdTomato+ at 4 months post i.v. treatment. N=2 technical replicates for each group. Mean+/−standard deviation shown. p-value by t-test. *p<0.05. (FIG. 10B) Gene editing frequency in non-hematopoietic organs/cells of the liver and (FIG. 10C) lung 16 weeks post in vivo treatment with CD117/LNP-Cre (1 μg and 5 μg) and control IgG/LNP-Cre (5 μg) assessment by flow cytometry. Shown mean+/−standard deviation (N=5 to 7); p-value calculated using Tukey's multiple comparison test after one-way ANOVA (p<0.05) for (FIG. 10A) and (FIG. 10B). Comparisons to control liver not shown. ****p<0.0001. (FIG. 10D) Frequency of cKit+ cells in the lung and frequency of gene editing in those cKit+ lung cells 6d after in vivo CD117/LNP-Cre treatment (3.5 μg) N=5. Shown mean+/−SD. (FIG. 10E) Gene editing frequency in non-hematopoietic organs/cells of the testis 16 weeks post in vivo treatment with CD117/LNP-Cre (5 μg and 1 μg) and control IgG/LNP-Cre (5 μg) assessment by flow cytometry. Shown mean+/−standard deviation (N=5 to 7); p-value calculated using Dunn's multiple comparison test after one-way ANOVA (p<0.05).

FIG. 11A and FIG. 11D depict data demonstrating the analyses of genome editing, cell viability and proliferation in human erythroid cells treated with CD117/LNP-ABE and CD117/LNP-sgRNA. (FIG. 11A) Representative sequences and (FIG. 11B) quantification of C editing of control unedited (top) and edited (bottom) genomic DNA extracted from SCD cells after treatment with anti-human CD117/LNP formulations carrying an adenine base editor and a sgRNA aimed at converting the pathogenic codon 6 (highlighted in blue, GAG,) to non-pathogenic variant (GCG) named HBB_{G-Makassar}. (FIG. 11C) Quantification of viability (AO/PI staining) from unedited (n=4) and edited (n=7) early erythroid progenitors cultured independently. (FIG. 11D) Proliferation rate of specimens in C calculated by measuring the cell count fold increase from DO to D7/8 in differentiation media. Shown is mean. P-value by Mann-Whitney; alpha=0.05.

FIG. 12A through FIG. 12D depict data demonstrating the base editing of the E6V sickle cell mutation with human CD117 targeted LNP. (FIG. 12A) Representative reverse-phase (RP) high-performance liquid chromatography (HPLC) chromatograms of in vitro differentiated sickle cell disease (SCD) erythroid progenitor lysates untreated (left) and after treatment with anti-human CD117 (hCD117)/LNP-NRCH Cas9 ABE-8e mRNA and hCD117/LNP gRNA (middle and right). Base editing yields nonpathogenic HBBG (β^{G}), which elutes before pathogenic HBB^{S} (β^{S}) and the a-globin protein (α). Percent shown is β^{G}/(β^{G}+β^{S})*100. (FIG. 12B) Representative images of sickling of in vitro-differentiated erythroid progenitors under hypoxic conditions at the treatments in (FIG. 12A). Arrowheads indicate sickled morphology. Scale bar, 20 mm. (FIG. 12C) Percentage of sickled cells from unedited and edited (varying mRNA doses) sickling assays. Data represent mean±SD of n=10 high-powered fields (hpf) (unedited specimens) and n=30 hpf (edited specimens). P values are reported from unpaired t test. ****P<0.0001. (FIG. 12D) Correlation of % β^{G} by RP-HPLC (protein) to base edited allele frequency (DNA).

FIG. 13A and FIG. 13C depict data demonstrating the identification of human PUMA as the most effective pro-apoptotic mRNA in HSC (FIG. 13A, left) Viability of mouse bone marrow cells 48 hours after in vitro treatment with CD117/LNP carrying pro-apoptotic mRNAs and GFP mRNA at escalating doses. Assessment with flow cytometry and 7-AAD stain. Shown mean+/−SEM (n=3). (FIG. 13A, right) Frequency of Lin−Scal+c-Kit− subset of bone marrow cells six days after in vitro treatment with LNP-mRNA. (FIG. 13B) Frequency of viable c-Kit+ cells, LSK cells, and SLAM (CD150+CD48-subset of LSK) cells two or six days after in vivo treatment of C57BL/6 mice with CD117/LNP-PUMA (n=3). Shown are mean+/−SEM. p-value calculated with Bonferroni's multiple comparison test after two-way ANOVA (p<0.05). *p<0.05. (FIG. 13C) Competitive transplantation schema for comparing residual engraftment capacity of CD45.2 bone marrow cells treated ex vivo with CD117/LNP-PUMA for 18 hours against varying ratios of untreated GFP+, CD45.2+ donor cells. Control groups of 50:50 mixture of untreated cells (GFP+ and GFP− cells) (group 1) and 100% CD117/LNP-PUMA treated cells without any untreated GFP+ cells (group 4). Recipients are lethally irradiated CD45.1+ mice.

FIG. 14A through FIG. 14C depict data demonstrating the HSC depletion and transplantation conditioning with CD117/LNPPUMA. (FIG. 14A to FIG. 14D) GFP+ granulocytes (FIG. 14A) and RBCs in peripheral blood (FIG. 14B), as well as percentage of GFP+CD45+ splenocytes (FIG. 14C) and BM cells (FIG. 14D) of C57BL/6 CD45.1 chimeras competitively transplanted with indicated proportion of GFP+C57BL/6 BM untreated and C57BL/6 (GFP−) BM treated with CD117/LNP-PUMA. Data represent mean±SD of n=4 (recipients of a 25:75 ratio of GFP:C57BL/6+CD117/LNP-PUMA BM), n=8 (recipients of a 50:50 ratio of GFP:C57BL/6+CD117/LNP-PUMA BM), and n=4 (recipients of a 50:50 ratio of GFP:C57BL/6 untreated BM) experimental animals per cohort. P values calculated by means of Dunnett's multiple comparison test after one-way ANOVA. ****P<0.0001. (FIG. 14E) Donor chimerism 4 months post-HSCT. Chimerism calculated as CD45.2%/(CD45.1%+CD45.2%). Data represent mean±SD of the same cohorts indicated in (FIG. 14A) to (FIG. 14C). One-way ANOVA not significant (P>0.05). (FIG. 14F) RBC, (FIG. 14G) granulocyte, and (FIG. 14H) hematopoietic cells of the BM, BM subsets, and spleen in recipients conditioned with 0.05 mg/kg CD117/LNP-PUMA and receiving 10×10⁶ GFP+C57BL/6 BM cells at 6.5 days after treatment. Data in (FIG. 14F) to (FIG. 14H) represent mean±SD of n=3 recipient animals. Levels of GFP+ granulocytes and RBCs in unconditioned controls (n=2) were nearly undetected (0.06±0.03 and 0.05±0.02, respectively) 2 months after BMT. (FIG. 14I) Persistence upon secondary transplantation of CD117/LNP-PUMA-conditioned GFP+ donor BM in lethally irradiated congenic mice. Data represent mean±SD of n=8 recipient animals generated from 3 primary chimeras.

FIG. 15A through FIG. 15C depict data demonstrating the imaging of mice and livers upon i.v. administration of CD117/LNP-Luc versus CD117/LNP-Luc-miRt formulations. Biodistribution upon i.v. injection of 1 μg of targeted LNP-mRNA expression in vivo by luminescence imaging at 24 hours. A representative sample set of mice (FIG. 15A) and corresponding livers dissected from these animals (FIG. 15B) were analyzed 5 min after the administration of D-luciferin. (FIG. 15C) Quantification of liver bioluminescence in FIG. 15B.

FIG. 16 depicts GFP+ cells after 1 and 2 months from BMT, post IV or IO injections with CD117/LNP3-PUMA.

FIG. 17 depicts the proportion of GFP+ cells in BL/6 mice preinjected with IP with Benadryl and preconditioned with 0.075 mg/kg of CD117/LNP3-PUMA or unconjugated LNP-PUMA particles prior to unrelated stem cell transplantation (USCT) with GFP donor BM.

FIG. 18 shows Top: Percentage of GFP+ red blood cells (Flow analyses) in a representative mouse (854-RR) preconditioned with ^{CD117}LNP-PUMA^{miR122} at 2.5 μg/mouse, using new protocol (pre/post treatments with Benadryl/Dexamethasone), followed by injection of healthy GPB BM; Middle and Bottom: Percentage of GFP+ white blood cells, including myeloid (Gr1), and lymphoid (B and T cells) (Flow analyses) in a representative mouse (854-RR) preconditioned with ^{CD117}LNP-PUMA^{miR122} at 2.5 μg/mouse (data at 4 month post BMT), using new protocol (pre/post treatments with Benadryl/Dexamethasone).

FIG. 19 shows that there is an improvement of BT features in thalassemic mice 854-RR and RL treated with GFP BM after ^{CD117}LNP-PUMA^{miR122} preconditioning: Hemoglobin, Hematocrit and RBC counts are increased, while reticulocyte % (a hallmark of BT) are reduced.

FIG. 20 shows that there is amelioration of morphology in RBC analyzed after erythroid Giemsa staining. Comparison between WT, BT and BT mice transplanted with GFP-BM after ^{CD117}LNP-PUMA^{miR122} preconditioning. In treated mouse (854RL), RBC's size, shape, color and number are improved, resembling features similar to those seeing in WT RBC

FIG. 21 shows a summary of % GFP+ red blood cells (Flow analyses) over 4 months in 3 cohorts: 1) mice preconditioned with ^{CD117}LNP-PUMA^{miR122} at 2.5 μg/mouse, using new protocol (pre/post treatments with Benadryl/Dexamethasone) (n=5, represented by solid circles, with 3 out of 5 animals up to 2 months); 2) mice preconditioned mobilized with a combination of bortezomib and plerixafor 18 hours prior to preconditioning with ^{CD117}LNP-PUMA^{miR122} at 2.5 μg/mouse, using new protocol (pre/post treatments with Benadryl/Dexamethasone) (n=2, represented by solid squares, 3 additional mice are in progress); 3) mice preconditioned with ^{CD117}LNP-PUMA^{miR122} at 3 consecutive doses of 1.5 μg/mouse, over 3 days (injected 24 hours apart) using new protocol (pre/post treatments with Benadryl/Dexamethasone) (n=3, represented by solid triangles, with 2 out of 3 animals followed up to 2 weeks), All animals have been injected with 10E+06 GFP+BM cells from donor healthy mice 6.5 days after ^{CD117}LNP-PUMA^{miR122} preconditioning.

### DETAILED DESCRIPTION

The present invention relates to compositions comprising CD117 targeted LNP molecules comprising a PUMA mRNA for preconditioning of Hematopoietic stem cells (HSCs).

The present invention also relates to methods of use of the compositions described herein in a non-genotoxic conditioning regiment for HSCs as well as methods of treating diseases or disorders in subjects including, but not limited to, bone marrow genetic defects. In some embodiments, the bone marrow genetic defect is leukemia, aplastic anemia, myeloproliferative disorders, an inherited bone marrow failure syndrome (IBMFS) such as Fanconi anemia, dyskeratosis congenital, Shwachman-Diamond syndrome, Diamond-Blackfan anemia, severe congenital neutropenia, a primary immunodeficiency such as X1-SCID and Wiskott-Aldrich syndrome, an erythroid disorder such as sickle cell disease (SCD), pyruvate kinase deficiency, or a lysosomal storage diseases such as Fabry disease and Pompe disease.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “adjuvant” as used herein means an agent that modifies or boosts the strength and longevity of a desired therapeutic response, and/or broadens the therapeutic response to a concomitantly administered agent.

The term “antibody,” as used herein, refers to an immunoglobulin molecule, which specifically binds with an antigen or epitope. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic-specificity determining variable regions of an intact antibody. Examples of antibody fragments include, but are not limited to, Fab, Fab′, F(ab′)₂, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. k and 1 light chains refer to the two major antibody light chain isotypes.

By the term “synthetic antibody” as used herein, is meant an antibody, which is generated using recombinant DNA technology, such as, for example, an antibody expressed by a bacteriophage. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art. The term should also be construed to mean an antibody, which has been generated by the synthesis of an RNA molecule encoding the antibody. The RNA molecule expresses an antibody protein, or an amino acid sequence specifying the antibody, wherein the RNA has been obtained by transcribing DNA (synthetic or cloned) or other technology, which is available and well known in the art.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

An “effective amount” as used herein, means an amount which provides a therapeutic or prophylactic benefit.

The term “physiologically effective dosage” refers to an amount of an agent that produces a measurable biologic or physiologic effect in the recipient subject that is related to the activity of the agent(s). The physiologically effective dosage will vary depending on the compound, the age, weight, etc., of the subject being administered the agent, and the biologic or physiologic effect being measured.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or CDNA.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) RNA, and viruses (e.g., lentiviruses, retroviruses, adenoviruses, and adeno-associated viruses) that incorporate the recombinant polynucleotide.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared ×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleosides (nucleobase bound to ribose or deoxyribose sugar via N-glycosidic linkage) are used. “A” refers to adenosine, “C” refers to cytidine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns. In addition, the nucleotide sequence may contain modified nucleosides that are capable of being translation by translational machinery in a cell. For example, an mRNA where all of the uridines have been replaced with pseudouridine, 1-methyl pseudouridine, or another modified nucleoside.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA or RNA sequences are contiguous and, where necessary to join two protein coding regions, in the same reading frame.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

In certain instances, the polynucleotide or nucleic acid of the invention is a “nucleoside-modified nucleic acid,” which refers to a nucleic acid comprising at least one modified nucleoside. A “modified nucleoside” refers to a nucleoside with a modification. For example, over one hundred different nucleoside modifications have been identified in RNA (Rozenski, et al., 1999, The RNA Modification Database: 1999 update. Nucl Acids Res 27:196-197).

In certain embodiments, “pseudouridine” refers, in another embodiment, to m¹acp³Y (1-methyl-3-(3-amino-3-carboxypropyl) pseudouridine. In another embodiment, the term refers to m¹Y (1-methylpseudouridine). In another embodiment, the term refers to Ym (2′-O-methylpseudouridine. In another embodiment, the term refers to m⁵D (5-methyldihydrouridine). In another embodiment, the term refers to m³Y (3-methylpseudouridine). In another embodiment, the term refers to a pseudouridine moiety that is not further modified. In another embodiment, the term refers to a monophosphate, diphosphate, or triphosphate of any of the above pseudouridines. In another embodiment, the term refers to any other pseudouridine known in the art. Each possibility represents a separate embodiment of the present invention.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence. For example, the promoter that is recognized by bacteriophage RNA polymerase and is used to generate the mRNA by in vitro transcription.

By the term “specifically binds,” as used herein with respect to an affinity ligand, in particular, an antibody, is meant an antibody which recognizes a specific antigen, but does not substantially recognize or bind other molecules in a sample. For example, an antibody that specifically binds to an antigen from one species may also bind to that antigen from one or more other species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “therapeutic” as used herein means a treatment and/or prophylaxis. A therapeutic effect is obtained by suppression, diminution, remission, or eradication of at least one sign or symptom of a disease or disorder.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

To “treat” a disease as the term is used herein, means to reduce the frequency or severity of at least one sign or symptom of a disease or disorder experienced by a subject.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Alkyl” refers to a straight or branched hydrocarbon chain radical consisting solely of carbon and hydrogen atoms, which is saturated or unsaturated (i.e., contains one or more double and/or triple bonds), having from one to twenty-four carbon atoms (C₁-C₂₄ alkyl), one to twelve carbon atoms (C₁-C₁₂ alkyl), one to eight carbon atoms (C₁-C₈alkyl) or one to six carbon atoms (C₁-C₆ alkyl) and which is attached to the rest of the molecule by a single bond, e.g., methyl, ethyl, n propyl, 1-methylethyl (iso propyl), n butyl, n pentyl, 1,1 dimethylethyl (t butyl), 3 methylhexyl, 2 methylhexyl, ethenyl, prop 1 enyl, but-1-enyl, pent-1-enyl, penta-1,4-dienyl, ethynyl, propynyl, butynyl, pentynyl, hexynyl, and the like. Unless specifically stated otherwise, an alkyl group is optionally substituted.

“Alkylene” or “alkylene chain” refers to a straight or branched divalent hydrocarbon chain linking the rest of the molecule to a radical group, consisting solely of carbon and hydrogen, which is saturated or unsaturated (i.e., contains one or more double (alkenylene) and/or triple bonds (alkynylene)), and having, for example, from one to twenty-four carbon atoms (C₁-C₂₄ alkylene), one to fifteen carbon atoms (C₁-C₁₅ alkylene), one to twelve carbon atoms (C₁-C₁₂ alkylene), one to eight carbon atoms (C₁-C₈ alkylene), one to six carbon atoms (C₁-C₆ alkylene), two to four carbon atoms (C₂-C₄ alkylene), one to two carbon atoms (C₁-C₂ alkylene), e.g., methylene, ethylene, propylene, n-butylene, ethenylene, propenylene, n-butenylene, propynylene, n-butynylene, and the like. The alkylene chain is attached to the rest of the molecule through a single or double bond and to the radical group through a single or double bond. The points of attachment of the alkylene chain to the rest of the molecule and to the radical group can be through one carbon or any two carbons within the chain. Unless stated otherwise specifically in the specification, an alkylene chain may be optionally substituted.

“Cycloalkyl” or “carbocyclic ring” refers to a stable non aromatic monocyclic or polycyclic hydrocarbon radical consisting solely of carbon and hydrogen atoms, which may include fused or bridged ring systems, having from three to fifteen carbon atoms, preferably having from three to ten carbon atoms, and which is saturated or unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, 7,7 dimethyl bicyclo[2.2.1]heptanyl, and the like. Unless specifically stated otherwise, a cycloalkyl group is optionally substituted.

“Cycloalkylene” is a divalent cycloalkyl group. Unless otherwise stated specifically in the specification, a cycloalkylene group may be optionally substituted.

“Heterocyclyl” or “heterocyclic ring” refers to a stable 3- to 18-membered non-aromatic ring radical which consists of two to twelve carbon atoms and from one to six heteroatoms selected from the group consisting of nitrogen, oxygen and sulfur. Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless specifically stated otherwise, a heterocyclyl group may be optionally substituted.

The term “substituted” used herein means any of the above groups (e.g., alkyl, cycloalkyl or heterocyclyl) wherein at least one hydrogen atom is replaced by a bond to a non-hydrogen atoms such as, but not limited to: a halogen atom such as F, Cl, Br, and I; oxo groups (═O); hydroxyl groups (—OH); alkoxy groups (—OR^{a}, where R^{a} is C₁-C₁₂ alkyl or cycloalkyl); carboxyl groups (—OC(═O)R^{a} or —C(—O) OR^{a}, where R^{a} is H, C₁-C₁₂ alkyl or cycloalkyl); amine groups (—NR^{a}R^{b}, where R^{a} and R^{b} are each independently H, C₁-C₁₂ alkyl or cycloalkyl); C₁-C₁₂ alkyl groups; and cycloalkyl groups. In some embodiments the substituent is a C₁-C₁₂ alkyl group. In other embodiments, the substituent is a cycloalkyl group. In other embodiments, the substituent is a halo group, such as fluoro. In other embodiments, the substituent is a oxo group. In other embodiments, the substituent is a hydroxyl group. In other embodiments, the substituent is an alkoxy group. In other embodiments, the substituent is a carboxyl group. In other embodiments, the substituent is an amine group.

“Optional” or “optionally” (e.g., optionally substituted) means that the subsequently described event of circumstances may or may not occur, and that the description includes instances where said event or circumstance occurs and instances in which it does not. For example, “optionally substituted alkyl” means that the alkyl radical may or may not be substituted and that the description includes both substituted alkyl radicals and alkyl radicals having no substitution.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Pro-Apoptotic mRNA Delivery

In one embodiment, the CD117-LNP comprises an mRNA molecule encoding a pro-apoptotic protein. In some embodiments, the pro-apoptotic protein is p53 upregulated modulator of apoptosis (PUMA), BCL2 Antagonist/Killer 1 (Bak-1), Caspase 3 (Cas-3) or Caspase 9 (Cas-9).

In one embodiment, the pro-apoptotic protein is PUMA. In one embodiment, the mRNA encodes a PUMA protein having the amino acid sequence: MARARQEGSSPEPVEGLARDSPRPFPLGRLMPSAVSCSLCEPGLPAAPAAPALLP AAYLCAPTAPPAVTAALGGPRWPGGHRSRPRGPRPDGPQPSLSPAQQHLESPVPS APEALAGGPTQAAPGVRVEEEEWAREIGAQLRRMADDLNAQYERRRQEEQHRH RPSPWRVMYNLFMGLLPLPRDPGAPEMEPN (SEQ ID NO:1), or a functional variant thereof, or a functional fragment thereof. In some embodiments, the functional variant or functional fragment of the PUMA protein retains the ability to induce apoptosis in a cell expressing the variant PUMA protein or PUMA protein fragment. In one embodiment, the PUMA mRNA comprises SEQ ID NO: 5 or a functional variant thereof, or a functional fragment thereof.

In one embodiment, the pro-apoptotic protein is Bak-1. In one embodiment, the mRNA encodes a Bak-1 protein, or a functional variant thereof, or a functional fragment thereof. In some embodiments, the functional variant or functional fragment of the Bak-1 protein retains the ability to induce apoptosis in a cell expressing the variant Bak-1 protein or Bak-1 protein fragment. In one embodiment, the Bak-1 mRNA comprises SEQ ID NO: 6 or a functional variant thereof, or a functional fragment thereof.

In one embodiment, the pro-apoptotic protein is Cas-3. In one embodiment, the mRNA encodes a Cas-3 protein, or a functional variant thereof, or a functional fragment thereof. In some embodiments, the functional variant or functional fragment of the Cas-3 protein retains the ability to induce apoptosis in a cell expressing the variant Cas-3 protein or Cas-3 protein fragment. In one embodiment, the Cas-3 mRNA comprises SEQ ID NO: 7 or a functional variant thereof, or a functional fragment thereof.

In one embodiment, the pro-apoptotic protein is Cas-7. In one embodiment, the mRNA encodes a Cas-7 protein, or a functional variant thereof, or a functional fragment thereof. In some embodiments, the functional variant or functional fragment of the Cas-7 protein retains the ability to induce apoptosis in a cell expressing the variant Cas-7 protein or Cas-7 protein fragment. In one embodiment, the Cas-7 mRNA comprises SEQ ID NO: 8 or a functional variant thereof, or a functional fragment thereof.

mRNA molecules include nucleotide oligomers containing modified backbones or non-natural inter-nucleoside linkages. Oligomers having modified backbones include those that retain a phosphorus atom in the backbone and those that do not have a phosphorus atom in the backbone. For the purposes of this disclosure, modified oligonucleotides that do not have a phosphorus atom in their internucleoside backbone are also considered to be nucleotide oligomers. Nucleotide oligomers that have modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates, thionophosphoramidates, thionoalkylphosphonates, thionoalkylphosphotriest-ers, and boranophosphates. Various salts, mixed salts and free acid forms are also included.

### In Vitro Transcribed RNA

In one embodiment, the composition of the invention comprises in vitro transcribed (IVT) RNA. In one embodiment, the composition of the invention comprises in vitro transcribed (IVT) RNA encoding a therapeutic protein. In one embodiment, the composition of the invention comprises IVT RNA encoding a plurality of therapeutic proteins.

In one embodiment, an IVT RNA can be introduced to a cell as a form of transient transfection. The RNA is produced by in vitro transcription using a plasmid DNA template generated synthetically. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. In one embodiment, the desired template for in vitro transcription is a therapeutic protein, as described elsewhere herein.

In one embodiment, the DNA to be used for PCR contains an open reading frame. The DNA can be from a naturally occurring DNA sequence from the genome of an organism. In one embodiment, the DNA is a full-length gene of interest of a portion of a gene. The gene can include some or all of the 5′ and/or 3′ untranslated regions (UTRs). The gene can include exons and introns. In one embodiment, the DNA to be used for PCR is a human gene. In another embodiment, the DNA to be used for PCR is a human gene including the 5′ and 3′ UTRs. In another embodiment, the DNA to be used for PCR is a gene from a pathogenic or commensal organism, including bacteria, viruses, parasites, and fungi. In another embodiment, the DNA to be used for PCR is from a pathogenic or commensal organism, including bacteria, viruses, parasites, and fungi, including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

Genes that can be used as sources of DNA for PCR include genes that encode a PUMA protein or a variant thereof that induces apoptosis in a targeted HSC.

In various embodiments, a plasmid is used to generate a template for in vitro transcription of RNA which is used for transfection.

Chemical structures with the ability to promote stability and/or translation efficiency may also be used. The RNA preferably has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of RNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many RNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the RNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one preferred embodiment, the promoter is a T7 RNA polymerase promoter, as described elsewhere herein. Other useful promoters include, but are not limited to, T3 and SP6 RNA polymerase promoters. Consensus nucleotide sequences for T7, T3 and SP6 promoters are known in the art.

In a preferred embodiment, the RNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized RNA which is effective in eukaryotic transfection when it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of polyA/T stretches into a DNA template is molecular cloning. However polyA/T sequence integrated into plasmid DNA can cause plasmid instability, which can be ameliorated through the use of recombination incompetent bacterial cells for plasmid propagation.

Poly(A) tails of RNAs can be further extended following in vitro transcription with the use of a poly(A) polymerase, such as E. coli polyA polymerase (E-PAP) or yeast polyA polymerase. In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase RNA stability. Such attachment can contain modified/artificial nucleotides, aptamers and other compounds. For example, ATP analogs can be incorporated into the poly(A)tail using poly(A) polymerase. ATP analogs can further increase the stability of the RNA.

5′ caps on also provide stability to RNA molecules. In a preferred embodiment, RNAs produced by the methods to include a 5′ cap1 structure. Such cap1 structure can be generated using Vaccinia capping enzyme and 2′-O-methyltransferase enzymes (CellScript, Madison, WI). Alternatively, 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

### Nucleoside-Modified RNA

In one embodiment, the composition of the present invention comprises a nucleoside-modified nucleic acid. In one embodiment, the composition of the invention comprises a nucleoside-modified RNA encoding a PUMA protein.

For example, in one embodiment, the composition comprises a nucleoside-modified RNA. In one embodiment, the composition comprises a nucleoside-modified mRNA. Nucleoside-modified mRNA have particular advantages over non-modified mRNA, including for example, increased stability, low or absent innate immunogenicity, and enhanced translation. Nucleoside-modified mRNA useful in the present invention is further described in U.S. Pat. No. 8,278,036, which is incorporated by reference herein in its entirety.

In certain instances, expressing a protein by delivering the encoding mRNA has many benefits over methods that use protein, plasmid DNA or viral vectors. During mRNA transfection, the coding sequence of the desired protein is the only substance delivered to cells, thus avoiding all the side effects associated with plasmid backbones, viral genes, and viral proteins. More importantly, unlike DNA- and viral-based vectors, the mRNA does not carry the risk of being incorporated into the genome and protein production starts immediately after mRNA delivery. For example, high levels of circulating proteins have been measured within 15 to 30 minutes of in vivo injection of the encoding mRNA. In certain embodiments, using mRNA rather than the protein also has many advantages. Half-lives of proteins in the circulation are often short, thus protein treatment would need frequent dosing, while mRNA provides a template for continuous protein production for several days. Purification of proteins is problematic and they can contain aggregates and other impurities that cause adverse effects (Kromminga and Schellekens, 2005, Ann NY Acad Sci 1050:257-265).

In certain embodiments, the nucleoside-modified RNA comprises the naturally occurring modified-nucleoside pseudouridine. In certain embodiments, inclusion of pseudouridine makes the mRNA more stable, non-immunogenic, and highly translatable (Kariko et al., 2008, Mol Ther 16:1833-1840; Anderson et al., 2010, Nucleic Acids Res 38:5884-5892; Anderson et al., 2011, Nucleic Acids Research 39:9329-9338; Kariko et al., 2011, Nucleic Acids Research 39: e142; Kariko et al., 2012, Mol Ther 20:948-953; Kariko et al., 2005, Immunity 23:165-175).

It has been demonstrated that the presence of modified nucleosides, including pseudouridines in RNA suppress their innate immunogenicity (Kariko et al., 2005, Immunity 23:165-175). Further, protein-encoding, in vitro-transcribed RNA containing pseudouridine can be translated more efficiently than RNA containing no or other modified nucleosides (Kariko et al., 2008, Mol Ther 16:1833-1840). Subsequently, it is shown that the presence of pseudouridine improves the stability of RNA (Anderson et al., 2011, Nucleic Acids Research 39:9329-9338) and abates both activation of PKR and inhibition of translation (Anderson et al., 2010, Nucleic Acids Res 38:5884-5892). A preparative HPLC purification procedure has been established that was critical to obtain pseudouridine-containing RNA that has superior translational potential and no innate immunogenicity (Kariko et al., 2011, Nucleic Acids Research 39: e142). Administering HPLC-purified, pseudourine-containing RNA coding for erythropoietin into mice and macaques resulted in a significant increase of serum EPO levels (Kariko et al., 2012, Mol Ther 20:948-953), thus confirming that pseudouridine-containing mRNA is suitable for in vivo protein therapy.

The present invention encompasses RNA, oligoribonucleotide, and polyribonucleotide molecules comprising pseudouridine or a modified nucleoside. In certain embodiments, the composition comprises an isolated nucleic acid, wherein the nucleic acid comprises a pseudouridine or a modified nucleoside. In certain embodiments, the composition comprises a vector, comprising an isolated nucleic acid, wherein the nucleic acid comprises a pseudouridine or a modified nucleoside.

In one embodiment, the nucleoside-modified RNA of the invention is IVT RNA, as described elsewhere herein. For example, in certain embodiments, the nucleoside-modified RNA is synthesized by T7 phage RNA polymerase. In another embodiment, the nucleoside-modified mRNA is synthesized by SP6 phage RNA polymerase. In another embodiment, the nucleoside-modified RNA is synthesized by T3 phage RNA polymerase.

In one embodiment, the modified nucleoside is m¹acp³Ψ (1-methyl-3-(3-amino-3-carboxypropyl) pseudouridine. In another embodiment, the modified nucleoside is m¹Ψ (1-methylpseudouridine). In another embodiment, the modified nucleoside is I'm (2′-O-methylpseudouridine. In another embodiment, the modified nucleoside is m⁵D (5-methyldihydrouridine). In another embodiment, the modified nucleoside is m³Ψ (3-methylpseudouridine). In another embodiment, the modified nucleoside is a pseudouridine moiety that is not further modified. In another embodiment, the modified nucleoside is a monophosphate, diphosphate, or triphosphate of any of the above pseudouridines. In another embodiment, the modified nucleoside is any other pseudouridine-like nucleoside known in the art.

In another embodiment, the nucleoside that is modified in the nucleoside-modified RNA the present invention is uridine (U). In another embodiment, the modified nucleoside is cytidine (C). In another embodiment, the modified nucleoside is adenosine (A). In another embodiment, the modified nucleoside is guanosine (G).

In another embodiment, the modified nucleoside of the present invention is m⁵C (5-methylcytidine). In another embodiment, the modified nucleoside is m⁵U (5-methyluridine). In another embodiment, the modified nucleoside is m⁶A (N⁶-methyladenosine). In another embodiment, the modified nucleoside is s²U (2-thiouridine). In another embodiment, the modified nucleoside is Ψ (pseudouridine). In another embodiment, the modified nucleoside is Um (2′-O-methyluridine).

In other embodiments, the modified nucleoside is m¹A (1-methyladenosine); m²A (2-methyladenosine); Am (2′-O-methyladenosine); ms²m⁶A (2-methylthio-N⁶-methyladenosine); i⁶A (N⁶-isopentenyladenosine); ms²¹⁶A (2-methylthio-N⁶isopentenyladenosine); io⁶A (N⁶-(cis-hydroxyisopentenyl) adenosine); ms²io⁶A (2-methylthio-N⁶-(cis-hydroxyisopentenyl) adenosine); g⁶A (N⁶-glycinylcarbamoyladenosine); t⁶A (N⁶-threonylcarbamoyladenosine); ms²io⁶A (2-methylthio-N6-threonyl carbamoyladenosine); m⁶t6A (N⁶-methyl-N⁶-threonylcarbamoyladenosine); hn⁶A (N⁶-hydroxynorvalylcarbamoyladenosine); ms²hn⁶A (2-methylthio-N⁶-hydroxynorvalyl carbamoyladenosine); Ar(p) (2′-O-ribosyladenosine (phosphate)); I (inosine); m¹I (1-methylinosine); m¹Im (1,2′-O-dimethylinosine); m³C (3-methylcytidine); Cm (2′-O-methylcytidine); s²C (2-thiocytidine); ac⁴C (N⁴-acetylcytidine); f⁵C (5-formylcytidine); m⁵Cm (5,2′-O-dimethylcytidine); ac⁴Cm (N⁴-acetyl-2′-O-methylcytidine); k²C (lysidine); m¹G (1-methylguanosine); m²G (N²-methylguanosine); m⁷G (7-methylguanosine); Gm (2′-O-methylguanosine); m²₂G (N²,N²-dimethylguanosine); m²Gm (N²,2′-O-dimethylguanosine); m²²Gm (N²,N²,2′-O-trimethylguanosine); Gr(p) (2′-O-ribosylguanosine (phosphate)); yW (wybutosine); o₂yW (peroxywybutosine); OHyW (hydroxywybutosine); OHyW* (undermodified hydroxywybutosine); imG (wyosine); mimG (methylwyosine); Q (queuosine); oQ (epoxyqueuosine); galQ (galactosyl-queuosine); manQ (mannosyl-queuosine); preQ₀ (7-cyano-7-deazaguanosine); preQ₁ (7-aminomethyl-7-deazaguanosine); G⁺ (archaeosine); D (dihydrouridine); m⁵Um (5,2′-O-dimethyluridine); s⁴U (4-thiouridine); m⁵s²U (5-methyl-2-thiouridine); s²Um (2-thio-2′-O-methyluridine); acp³U (3-(3-amino-3-carboxypropyl) uridine); ho⁵U (5-hydroxyuridine); mo⁵U (5-methoxyuridine); cmo⁵U (uridine 5-oxyacetic acid); mcmo⁵U (uridine 5-oxyacetic acid methyl ester); chm⁵U (5-(carboxyhydroxymethyl) uridine)); mchm⁵U (5-(carboxyhydroxymethyl) uridine methyl ester); mcm⁵U (5-methoxycarbonylmethyluridine); mcm⁵Um (5-methoxycarbonylmethyl-2′-O-methyluridine); mcm⁵s²U (5-methoxycarbonylmethyl-2-thiouridine); nm⁵s²U (5-aminomethyl-2-thiouridine); mnm⁵U (5-methylaminomethyluridine); mnm⁵s²U (5-methylaminomethyl-2-thiouridine); mnm⁵se²U (5-methylaminomethyl-2-selenouridine); ncm⁵U (5-carbamoylmethyluridine); ncm⁵Um (5-carbamoylmethyl-2′-O-methyluridine); cmnm⁵U (5-carboxymethylaminomethyluridine); cmnm⁵Um (5-carboxymethylaminomethyl-2′-O-methyluridine); cmnm⁵s²U (5-carboxymethylaminomethyl-2-thiouridine); m⁶₂A (N⁶,N⁶-dimethyladenosine); Im (2′-O-methylinosine); m⁴C (N⁴-methylcytidine); m⁴Cm (N⁴,2′-O-dimethylcytidine); hm⁵C (5-hydroxymethylcytidine); m³U (3-methyluridine); cm⁵U (5-carboxymethyluridine); m⁶Am (N⁶,2′-O-dimethyladenosine); m⁶₂Am (N⁶,N⁶,O-2′-trimethyladenosine); m^{2,7}G (N²,7-dimethylguanosine); m^{2,2,7}G (N²,N²,7-trimethylguanosine); m³Um (3,2′-O-dimethyluridine); m⁵D (5-methyldihydrouridine); f⁵Cm (5-formyl-2′-O-methylcytidine); m¹Gm (1,2′-O-dimethylguanosine); m¹Am (1,2′-O-dimethyladenosine); τm⁵U (5-taurinomethyluridine); τm⁵s²U (5-taurinomethyl-2-thiouridine)); imG-14 (4-demethylwyosine); imG2 (isowyosine); or ac⁶A (N⁶-acetyladenosine).

In another embodiment, a nucleoside-modified RNA of the present invention comprises a combination of 2 or more of the above modifications. In another embodiment, the nucleoside-modified RNA comprises a combination of 3 or more of the above modifications. In another embodiment, the nucleoside-modified RNA comprises a combination of more than 3 of the above modifications.

In another embodiment, between 0.1% and 100% of the residues in the nucleoside-modified of the present invention are modified (e.g. either by the presence of pseudouridine or a modified nucleoside base). In another embodiment, 0.1% of the residues are modified. In another embodiment, the fraction of modified residues is 0.2%. In another embodiment, the fraction is 0.3%. In another embodiment, the fraction is 0.4%. In another embodiment, the fraction is 0.5%. In another embodiment, the fraction is 0.6%. In another embodiment, the fraction is 0.8%. In another embodiment, the fraction is 1%. In another embodiment, the fraction is 1.5%. In another embodiment, the fraction is 2%. In another embodiment, the fraction is 2.5%. In another embodiment, the fraction is 3%. In another embodiment, the fraction is 4%. In another embodiment, the fraction is 5%. In another embodiment, the fraction is 6%. In another embodiment, the fraction is 8%. In another embodiment, the fraction is 10%. In another embodiment, the fraction is 12%. In another embodiment, the fraction is 14%. In another embodiment, the fraction is 16%. In another embodiment, the fraction is 18%. In another embodiment, the fraction is 20%. In another embodiment, the fraction is 25%. In another embodiment, the fraction is 30%. In another embodiment, the fraction is 35%. In another embodiment, the fraction is 40%. In another embodiment, the fraction is 45%. In another embodiment, the fraction is 50%. In another embodiment, the fraction is 60%. In another embodiment, the fraction is 70%. In another embodiment, the fraction is 80%. In another embodiment, the fraction is 90%. In another embodiment, the fraction is 100%.

In another embodiment, the fraction is less than 5%. In another embodiment, the fraction is less than 3%. In another embodiment, the fraction is less than 1%. In another embodiment, the fraction is less than 2%. In another embodiment, the fraction is less than 4%. In another embodiment, the fraction is less than 6%. In another embodiment, the fraction is less than 8%. In another embodiment, the fraction is less than 10%. In another embodiment, the fraction is less than 12%. In another embodiment, the fraction is less than 15%. In another embodiment, the fraction is less than 20%. In another embodiment, the fraction is less than 30%. In another embodiment, the fraction is less than 40%. In another embodiment, the fraction is less than 50%. In another embodiment, the fraction is less than 60%. In another embodiment, the fraction is less than 70%.

In another embodiment, 0.1% of the residues of a given nucleoside (i.e., uridine, cytidine, guanosine, or adenosine) are modified. In another embodiment, the fraction of the given nucleotide that is modified is 0.2%. In another embodiment, the fraction is 0.3%. In another embodiment, the fraction is 0.4%. In another embodiment, the fraction is 0.5%. In another embodiment, the fraction is 0.6%. In another embodiment, the fraction is 0.8%. In another embodiment, the fraction is 1%. In another embodiment, the fraction is 1.5%. In another embodiment, the fraction is 2%. In another embodiment, the fraction is 2.5%. In another embodiment, the fraction is 3%. In another embodiment, the fraction is 4%. In another embodiment, the fraction is 5%. In another embodiment, the fraction is 6%. In another embodiment, the fraction is 8%. In another embodiment, the fraction is 10%. In another embodiment, the fraction is 12%. In another embodiment, the fraction is 14%. In another embodiment, the fraction is 16%. In another embodiment, the fraction is 18%. In another embodiment, the fraction is 20%. In another embodiment, the fraction is 25%. In another embodiment, the fraction is 30%. In another embodiment, the fraction is 35%. In another embodiment, the fraction is 40%. In another embodiment, the fraction is 45%. In another embodiment, the fraction is 50%. In another embodiment, the fraction is 60%. In another embodiment, the fraction is 70%. In another embodiment, the fraction is 80%. In another embodiment, the fraction is 90%. In another embodiment, the fraction is 100%.

In another embodiment, the fraction of the given nucleotide that is modified is less than 8%. In another embodiment, the fraction is less than 10%. In another embodiment, the fraction is less than 5%. In another embodiment, the fraction is less than 3%. In another embodiment, the fraction is less than 1%. In another embodiment, the fraction is less than 2%. In another embodiment, the fraction is less than 4%. In another embodiment, the fraction is less than 6%. In another embodiment, the fraction is less than 12%. In another embodiment, the fraction is less than 15%. In another embodiment, the fraction is less than 20%. In another embodiment, the fraction is less than 30%. In another embodiment, the fraction is less than 40%. In another embodiment, the fraction is less than 50%. In another embodiment, the fraction is less than 60%. In another embodiment, the fraction is less than 70%.

In another embodiment, a nucleoside-modified RNA of the present invention is translated in the cell more efficiently than an unmodified RNA molecule with the same sequence. In another embodiment, the nucleoside-modified RNA exhibits enhanced ability to be translated by a target cell. In another embodiment, translation is enhanced by a factor of 2-fold relative to its unmodified counterpart. In another embodiment, translation is enhanced by a 3-fold factor. In another embodiment, translation is enhanced by a 5-fold factor. In another embodiment, translation is enhanced by a 7-fold factor. In another embodiment, translation is enhanced by a 10-fold factor. In another embodiment, translation is enhanced by a 15-fold factor. In another embodiment, translation is enhanced by a 20-fold factor. In another embodiment, translation is enhanced by a 50-fold factor. In another embodiment, translation is enhanced by a 100-fold factor. In another embodiment, translation is enhanced by a 200-fold factor. In another embodiment, translation is enhanced by a 500-fold factor. In another embodiment, translation is enhanced by a 1000-fold factor. In another embodiment, translation is enhanced by a 2000-fold factor. In another embodiment, the factor is 10-1000-fold. In another embodiment, the factor is 10-100-fold. In another embodiment, the factor is 10-200-fold. In another embodiment, the factor is 10-300-fold. In another embodiment, the factor is 10-500-fold. In another embodiment, the factor is 20-1000-fold. In another embodiment, the factor is 30-1000-fold. In another embodiment, the factor is 50-1000-fold. In another embodiment, the factor is 100-1000-fold. In another embodiment, the factor is 200-1000-fold. In another embodiment, translation is enhanced by any other significant amount or range of amounts.

### Inhibitory Domain

In some embodiments, the mRNA molecule includes a modification of the 3′UTR or the 5′UTR to include an inhibitory domain to prevent expression of the mRNA molecule in one or more non-targeted tissue types.

In some embodiments, the PUMA mRNA molecule is modified to contain a modification of the 3′UTR of the therapeutic mRNA molecule to include a miR-122 binding domain. miR-122 is expressed in liver cells and inhibits translation of genes which include a miR-122 binding site.

### Delivery Vehicle

In some embodiments, the delivery vehicle is a colloidal dispersion system, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

The use of lipid formulations is contemplated for the introduction of the at least one agent into a host cell (in vitro, ex vivo or in vivo). In another aspect, the at least one agent may be associated with a lipid. The at least one agent associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/nucleic acid or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Chol”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5:505-10). However, compositions that have different structures in solution than the normal vesicular structure are also encompassed. For example, the lipids may assume a micellar structure or merely exist as nonuniform aggregates of lipid molecules. Also contemplated are lipofectamine-agent complexes. In one embodiment, delivery of the at least one agent comprises any suitable delivery method, including exemplary delivery methods described elsewhere herein. In certain embodiments, delivery of the at least one agent to a subject comprises mixing the at least one agent with a transfection reagent prior to the step of contacting. In another embodiment, a method of the present invention further comprises administering the at least one agent together with the transfection reagent. In another embodiment, the transfection reagent is a cationic lipid reagent.

In another embodiment, the transfection reagent is a lipid-based transfection reagent. In another embodiment, the transfection reagent is a protein-based transfection reagent. In another embodiment, the transfection reagent is a polyethyleneimine based transfection reagent. In another embodiment, the transfection reagent is calcium phosphate. In another embodiment, the transfection reagent is Lipofectin®, Lipofectamine®, or TransIT®. In another embodiment, the transfection reagent is any other transfection reagent known in the art.

In another embodiment, the transfection reagent forms a liposome. Liposomes, in another embodiment, increase intracellular stability, increase uptake efficiency and improve biological activity. In another embodiment, liposomes are hollow spherical vesicles composed of lipids arranged in a similar fashion as those lipids which make up the cell membrane. In some embodiments, the liposomes comprise an internal aqueous space for entrapping water-soluble compounds. In another embodiment, liposomes can deliver the at least one agent to cells in an active form.

In one embodiment, the composition comprises a lipid nanoparticle (LNP) and at least one agent.

The term “lipid nanoparticle” refers to a particle having at least one dimension on the order of nanometers (e.g., 1-1,000 nm) which includes one or more lipids. In various embodiments, the particle includes a lipid of Formula (I), (II) or (III). In some embodiments, lipid nanoparticles are included in a formulation comprising at least one agent as described herein. In some embodiments, such lipid nanoparticles comprise a cationic lipid (e.g., a lipid of Formula (I), (II) or (III)) and one or more excipient selected from neutral lipids, charged lipids, steroids and polymer conjugated lipids (e.g., a pegylated lipid such as a pegylated lipid of structure (IV), such as compound IVa). In some embodiments, the at least one agent is encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells e.g. an adverse immune response.

In various embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In one embodiment, the lipid nanoparticles have a mean diameter of about 83 nm. In one embodiment, the lipid nanoparticles have a mean diameter of about 102 nm. In one embodiment, the lipid nanoparticles have a mean diameter of about 103 nm. In some embodiments, the lipid nanoparticles are substantially non-toxic. In certain embodiments, the at least one agent, when present in the lipid nanoparticles, is resistant in aqueous solution to degradation by intra- or intercellular enzymes

The LNP may comprise any lipid capable of forming a particle to which the at least one agent is attached, or in which the at least one agent is encapsulated. The term “lipid” refers to a group of organic compounds that are derivatives of fatty acids (e.g., esters) and are generally characterized by being insoluble in water but soluble in many organic solvents. Lipids are usually divided in at least three classes: (1) “simple lipids” which include fats and oils as well as waxes; (2) “compound lipids” which include phospholipids and glycolipids; and (3) “derived lipids” such as steroids.

In one embodiment, the LNP comprises one or more cationic lipids, and one or more stabilizing lipids. Stabilizing lipids include neutral lipids and pegylated lipids.

In one embodiment, the LNP comprises a cationic lipid. As used herein, the term “cationic lipid” refers to a lipid that is cationic or becomes cationic (protonated) as the pH is lowered below the pK of the ionizable group of the lipid, but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease.

In certain embodiments, the cationic lipid comprises any of a number of lipid species which carry a net positive charge at a selective pH, such as physiological pH. Such lipids include, but are not limited to, N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3-dioleoyloxy)propyl)-N,N,N-trimethylammonium chloride (DOTAP); 3-(N—(N′,N′-dimethylaminoethane)-carbamoyl) cholesterol (DC-Chol), N-(1-(2,3-dioleoyloxy)propyl)-N-2-(sperminecarboxamido)ethyl)-N,N-dimethylammonium trifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy)propylamine (DODMA), and N-(1,2-dimyristyloxyprop-3-yl)-N,N-dimethyl-N-hydroxyethyl ammonium bromide (DMRIE). Additionally, a number of commercial preparations of cationic lipids are available which can be used in the present invention. These include, for example, LIPOFECTIN® (commercially available cationic liposomes comprising DOTMA and 1,2-dioleoyl-sn-3-phosphoethanolamine (DOPE), from GIBCO/BRL, Grand Island, N.Y.); LIPOFECTAMINE® (commercially available cationic liposomes comprising N-(1-(2,3-dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.). The following lipids are cationic and have a positive charge at below physiological pH: DODAP, DODMA, DMDMA, 1,2-dilinoleyloxy-N,N-dimethylaminopropane (DLinDMA), 1,2-dilinolenyloxy-N,N-dimethylaminopropane (DLenDMA).

In one embodiment, the cationic lipid is an amino lipid. Suitable amino lipids useful in the invention include those described in WO 2012/016184, incorporated herein by reference in its entirety. Representative amino lipids include, but are not limited to, 1,2-dilinoleyoxy-3-(dimethylamino) acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA·Cl), 1,2-dilinoleoyl-3-trimethylaminopropane chloride salt (DLin-TAP·Cl), 1,2-dilinoleyloxy-3-(N-methylpiperazino) propane (DLin-MPZ), 3-(N,N-dilinoleylamino)-1,2-propanediol (DLinAP), 3-(N,N-dioleylamino)-1,2-propanediol (DOAP), 1,2-dilinoleyloxo-3-(2-N,N-dimethylamino) ethoxypropane (DLin-EG-DMA), and 2,2-dilinoleyl-4-dimethylaminomethyl-[1,3]-dioxolane (DLin-K-DMA).

Suitable amino lipids include those having the formula:

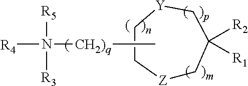

wherein R₁ and R₂ are either the same or different and independently optionally substituted C₁₀-C₂₄ alkyl, optionally substituted C₁₀-C₂₄ alkenyl, optionally substituted C₁₀-C₂₄ alkynyl, or optionally substituted C₁₀-C₂₄ acyl;
R₃ and R₄ are either the same or different and independently optionally substituted C₁-C₆ alkyl, optionally substituted C₂-C₆ alkenyl, or optionally substituted C₂-C₆ alkynyl or R₃ and R₄ may join to form an optionally substituted heterocyclic ring of 4 to 6 carbon atoms and 1 or 2 heteroatoms chosen from nitrogen and oxygen;
R₅ is either absent or present and when present is hydrogen or C₁-C₆ alkyl;
m, n, and p are either the same or different and independently either 0 or 1 with the proviso that m, n, and p are not simultaneously 0;
q is 0, 1, 2, 3, or 4; and
Y and Z are either the same or different and independently O, S, or NH.

In one embodiment, R₁ and R₂ are each linoleyl, and the amino lipid is a dilinoleyl amino lipid. In one embodiment, the amino lipid is a dilinoleyl amino lipid.

A representative useful dilinoleyl amino lipid has the formula:

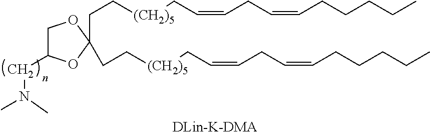

wherein n is 0, 1, 2, 3, or 4.

In one embodiment, the cationic lipid is a DLin-K-DMA. In one embodiment, the cationic lipid is DLin-KC2-DMA (DLin-K-DMA above, wherein n is 2).

In one embodiment, the cationic lipid component of the LNPs has the structure of Formula (I):

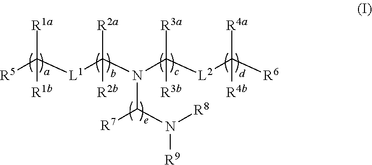

or a pharmaceutically acceptable salt, tautomer, prodrug or stereoisomer thereof, wherein:
L¹ and L² are each independently —O(C═O)—, —(C—O)O— or a carbon-carbon double bond;
R^{1a} and R^{1b} are, at each occurrence, independently either (a) H or C₁-C₁₂ alkyl, or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either (a) H or C₁-C₁₂ alkyl, or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a) H or C₁-C₁₂ alkyl, or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either (a) H or C₁-C₁₂ alkyl, or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently methyl or cycloalkyl;
R⁷ is, at each occurrence, independently H or C₁-C₁₂ alkyl;
R⁸ and R⁹ are each independently C₁-C₁₂ alkyl; or R⁸ and R⁹, together with the nitrogen atom to which they are attached, form a 5, 6 or 7-membered heterocyclic ring comprising one nitrogen atom;
a and d are each independently an integer from 0 to 24;
b and c are each independently an integer from 1 to 24; and
e is 1 or 2.

In certain embodiments of Formula (I), at least one of R^{1a}, R^{2a}, R^{3a} or R^{4a} is C₁-C₁₂ alkyl, or at least one of L¹ or L² is —O(C═O)— or —(C—O)O—. In other embodiments, R^{1a} and R^{1b} are not isopropyl when a is 6 or n-butyl when a is 8.

In still further embodiments of Formula (I), at least one of R^{1a}, R^{2a}, R^{3a} or R^{4a} is C₁-C₁₂ alkyl, or at least one of L¹ or L² is —O(C═O)— or —(C═O)O—; and

R^{1a} and R^{1b} are not isopropyl when a is 6 or n-butyl when a is 8.

In other embodiments of Formula (I), R⁸ and R⁹ are each independently unsubstituted C₁-C₁₂ alkyl; or R⁸ and R⁹, together with the nitrogen atom to which they are attached, form a 5, 6 or 7-membered heterocyclic ring comprising one nitrogen atom; In certain embodiments of Formula (I), any one of L¹ or L² may be —O(C═O)— or a carbon-carbon double bond. L¹ and L² may each be —O(C—O)— or may each be a carbon-carbon double bond.

In some embodiments of Formula (I), one of L¹ or L² is —O(C—O)—. In other embodiments, both L¹ and L² are —O(C—O)—.

In some embodiments of Formula (I), one of L¹ or L² is —(C═O)O—. In other embodiments, both L¹ and L² are —(C—O)O—.

In some other embodiments of Formula (I), one of L¹ or L² is a carbon-carbon double bond. In other embodiments, both L¹ and L² are a carbon-carbon double bond.

In still other embodiments of Formula (I), one of L¹ or L² is —O(C═O)— and the other of L¹ or L² is —(C═O)O—. In more embodiments, one of L¹ or L² is —O(C═O)— and the other of L¹ or L² is a carbon-carbon double bond. In yet more embodiments, one of L¹ or L² is —(C═O)O— and the other of L¹ or L² is a carbon-carbon double bond.

It is understood that “carbon-carbon” double bond, as used throughout the specification, refers to one of the following structures:

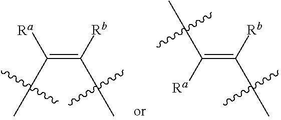

wherein R^{a} and R^{b} are, at each occurrence, independently H or a substituent. For example, in some embodiments R^{a} and R^{b} are, at each occurrence, independently H, C₁-C₁₂ alkyl or cycloalkyl, for example H or C₁-C₁₂ alkyl.

In other embodiments, the lipid compounds of Formula (I) have the following structure (Ia):

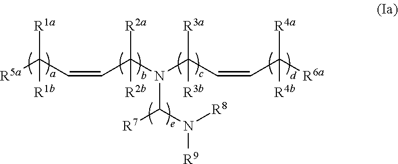

In other embodiments, the lipid compounds of Formula (I) have the following structure (Ib):

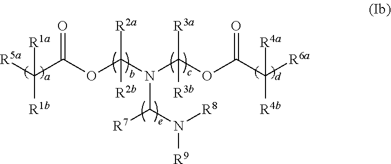

In yet other embodiments, the lipid compounds of Formula (I) have the

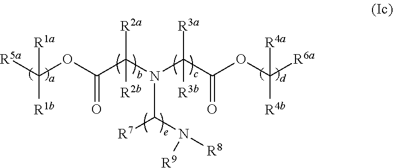

In certain embodiments of the lipid compound of Formula (I), a, b, c and d are each independently an integer from 2 to 12 or an integer from 4 to 12. In other embodiments, a, b, c and d are each independently an integer from 8 to 12 or 5 to 9. In some certain embodiments, a is 0. In some embodiments, a is 1. In other embodiments, a is 2. In more embodiments, a is 3. In yet other embodiments, a is 4. In some embodiments, a is 5. In other embodiments, a is 6. In more embodiments, a is 7. In yet other embodiments, a is 8. In some embodiments, a is 9. In other embodiments, a is 10. In more embodiments, a is 11. In yet other embodiments, a is 12. In some embodiments, a is 13. In other embodiments, a is 14. In more embodiments, a is 15. In yet other embodiments, a is 16.

In some other embodiments of Formula (I), b is 1. In other embodiments, b is 2. In more embodiments, b is 3. In yet other embodiments, b is 4. In some embodiments, b is 5. In other embodiments, b is 6. In more embodiments, b is 7. In yet other embodiments, b is 8. In some embodiments, b is 9. In other embodiments, b is 10. In more embodiments, b is 11. In yet other embodiments, b is 12. In some embodiments, b is 13. In other embodiments, b is 14. In more embodiments, b is 15. In yet other embodiments, b is 16.

In some more embodiments of Formula (I), c is 1. In other embodiments, c is 2. In more embodiments, c is 3. In yet other embodiments, c is 4. In some embodiments, c is 5. In other embodiments, c is 6. In more embodiments, c is 7. In yet other embodiments, c is 8. In some embodiments, c is 9. In other embodiments, c is 10. In more embodiments, c is 11. In yet other embodiments, c is 12. In some embodiments, c is 13. In other embodiments, c is 14. In more embodiments, c is 15. In yet other embodiments, c is 16.

In some certain other embodiments of Formula (I), dis 0. In some embodiments, dis 1. In other embodiments, dis 2. In more embodiments, d is 3. In yet other embodiments, dis 4. In some embodiments, d is 5. In other embodiments, d is 6. In more embodiments, dis 7. In yet other embodiments, d is 8. In some embodiments, d is 9. In other embodiments, dis 10. In more embodiments, dis 11. In yet other embodiments, d is 12. In some embodiments, dis 13. In other embodiments, dis 14. In more embodiments, dis 15. In yet other embodiments, dis 16.

In some other various embodiments of Formula (I), a and d are the same. In some other embodiments, b and c are the same. In some other specific embodiments, a and d are the same and b and c are the same.

The sum of a and b and the sum of c and d in Formula (I) are factors which may be varied to obtain a lipid of Formula (I) having the desired properties. In one embodiment, a and b are chosen such that their sum is an integer ranging from 14 to 24. In other embodiments, c and d are chosen such that their sum is an integer ranging from 14 to 24. In further embodiment, the sum of a and b and the sum of c and d are the same. For example, in some embodiments the sum of a and b and the sum of c and d are both the same integer which may range from 14 to 24. In still more embodiments, a, b, c and d are selected such the sum of a and b and the sum of c and d is 12 or greater.

In some embodiments of Formula (I), e is 1. In other embodiments, e is 2.

The substituents at R^{1a}, R^{2a}, R^{3a} and R^{4a} of Formula (I) are not particularly limited. In certain embodiments R^{1a}, R^{2a}, R^{3a} and R^{4a} are H at each occurrence. In certain other embodiments at least one of R^{1a}, R^{2a}, R^{3a} and R^{4a} is C₁-C₁₂ alkyl. In certain other embodiments at least one of R^{1a}, R^{2a}, R^{3a} and R^{4a} is C₁-C₈ alkyl. In certain other embodiments at least one of R^{1a}, R^{2a}, R^{3a} and R^{4a} is C₁-C₆ alkyl. In some of the foregoing embodiments, the C₁-C₈alkyl is methyl, ethyl, n-propyl, iso-propyl, n-butyl, iso-butyl, tert-butyl, n-hexyl or n-octyl.

In certain embodiments of Formula (I), R^{1a}, R^{1b}, R^{4a} and R^{4b} are C₁-C₁₂ alkyl at each occurrence.

In further embodiments of Formula (I), at least one of R^{1b}, R^{2b}, R^{3b} and R^{4b} is H or R^{1b}, R^{2b}, R^{3b} and R^{4b} are H at each occurrence.

In certain embodiments of Formula (I), R^{1b} together with the carbon atom to which it is bound is taken together with an adjacent R^{1b} and the carbon atom to which it is bound to form a carbon-carbon double bond. In other embodiments of the foregoing R^{4b} together with the carbon atom to which it is bound is taken together with an adjacent R^{4b} and the carbon atom to which it is bound to form a carbon-carbon double bond.

The substituents at R5 and R⁶ of Formula (I) are not particularly limited in the foregoing embodiments. In certain embodiments one or both of R⁵ or R⁶ is methyl. In certain other embodiments one or both of R5 or R⁶ is cycloalkyl for example cyclohexyl. In these embodiments the cycloalkyl may be substituted or not substituted. In certain other embodiments the cycloalkyl is substituted with C₁-C₁₂ alkyl, for example tert-butyl.

The substituents at R⁷ are not particularly limited in the foregoing embodiments of Formula (I). In certain embodiments at least one R⁷ is H. In some other embodiments, R⁷ is H at each occurrence. In certain other embodiments R⁷ is C₁-C₁₂ alkyl.

In certain other of the foregoing embodiments of Formula (I), one of R⁸ or R⁹ is methyl. In other embodiments, both R⁸ and R9 are methyl.

In some different embodiments of Formula (I), R⁸ and R9, together with the nitrogen atom to which they are attached, form a 5, 6 or 7-membered heterocyclic ring. In some embodiments of the foregoing, R⁸ and R⁹, together with the nitrogen atom to which they are attached, form a 5-membered heterocyclic ring, for example a pyrrolidinyl ring.

In various different embodiments, exemplary lipid of Formula (I) can include

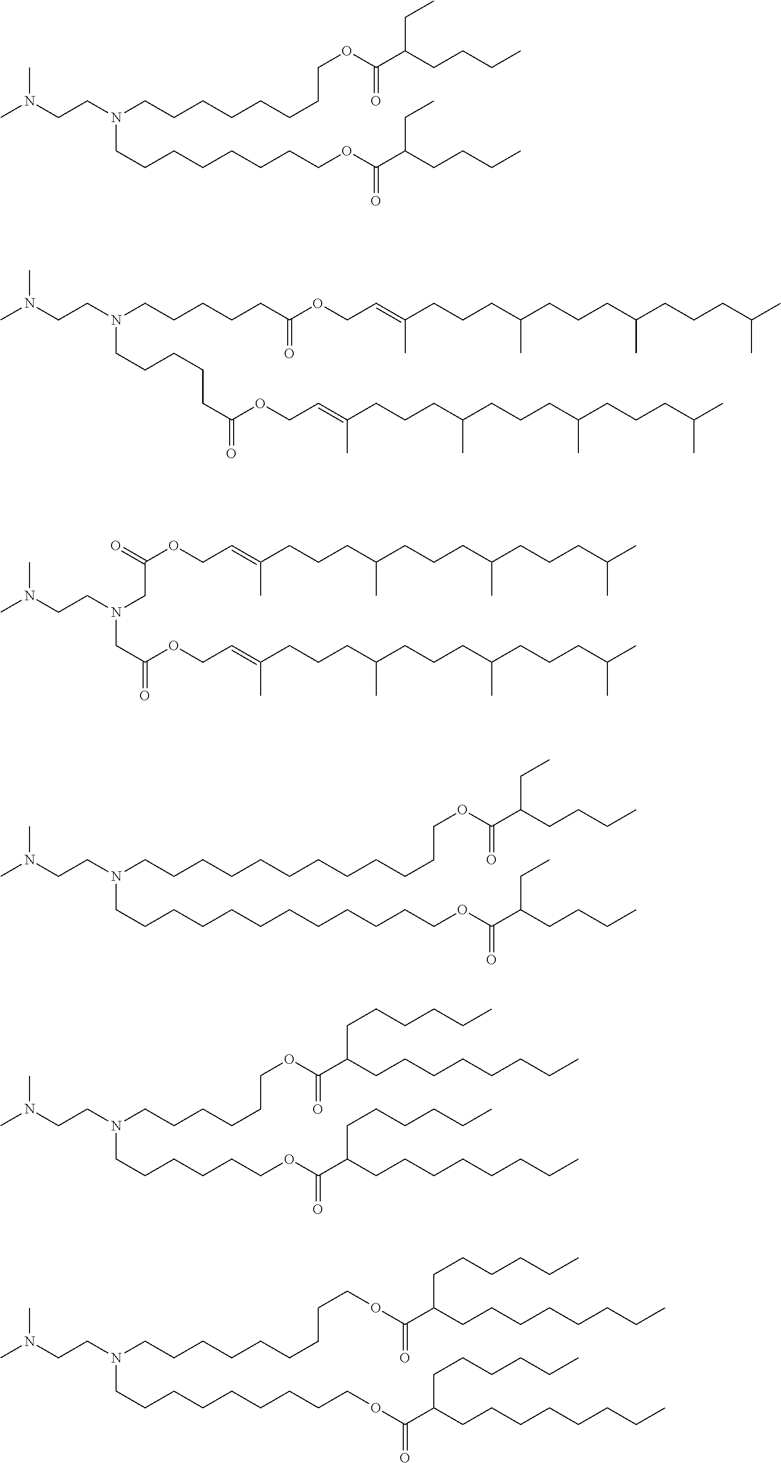

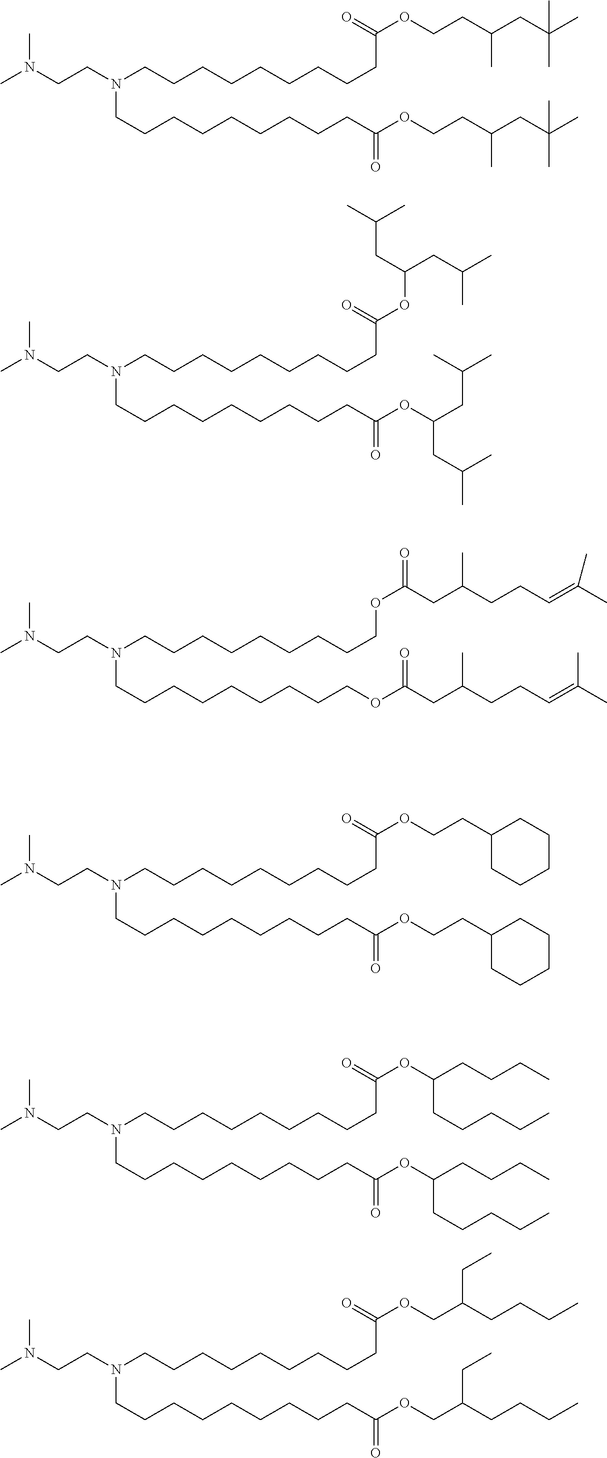

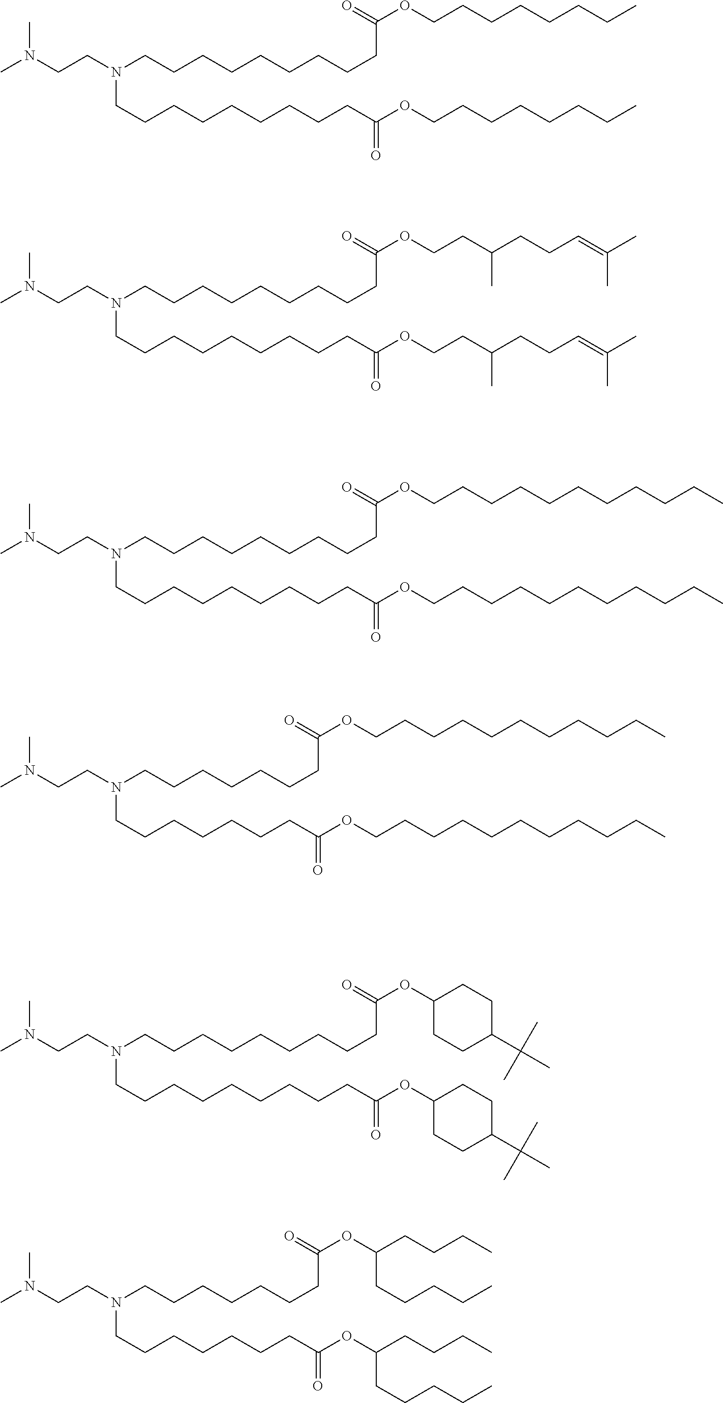

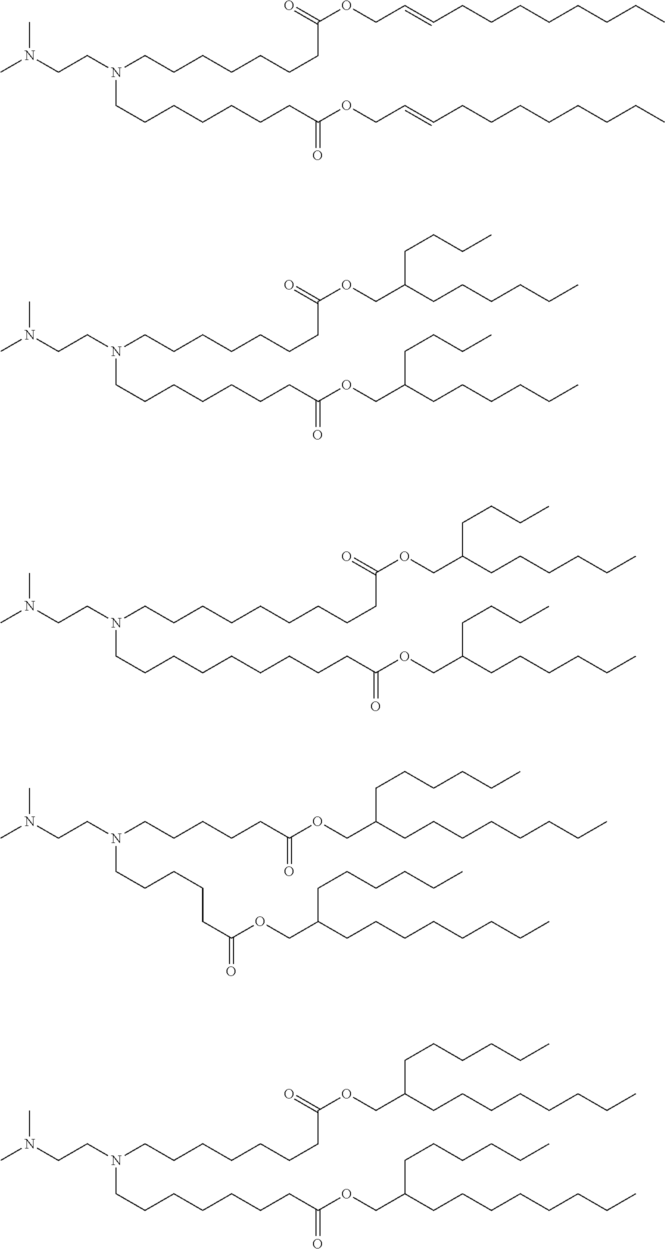

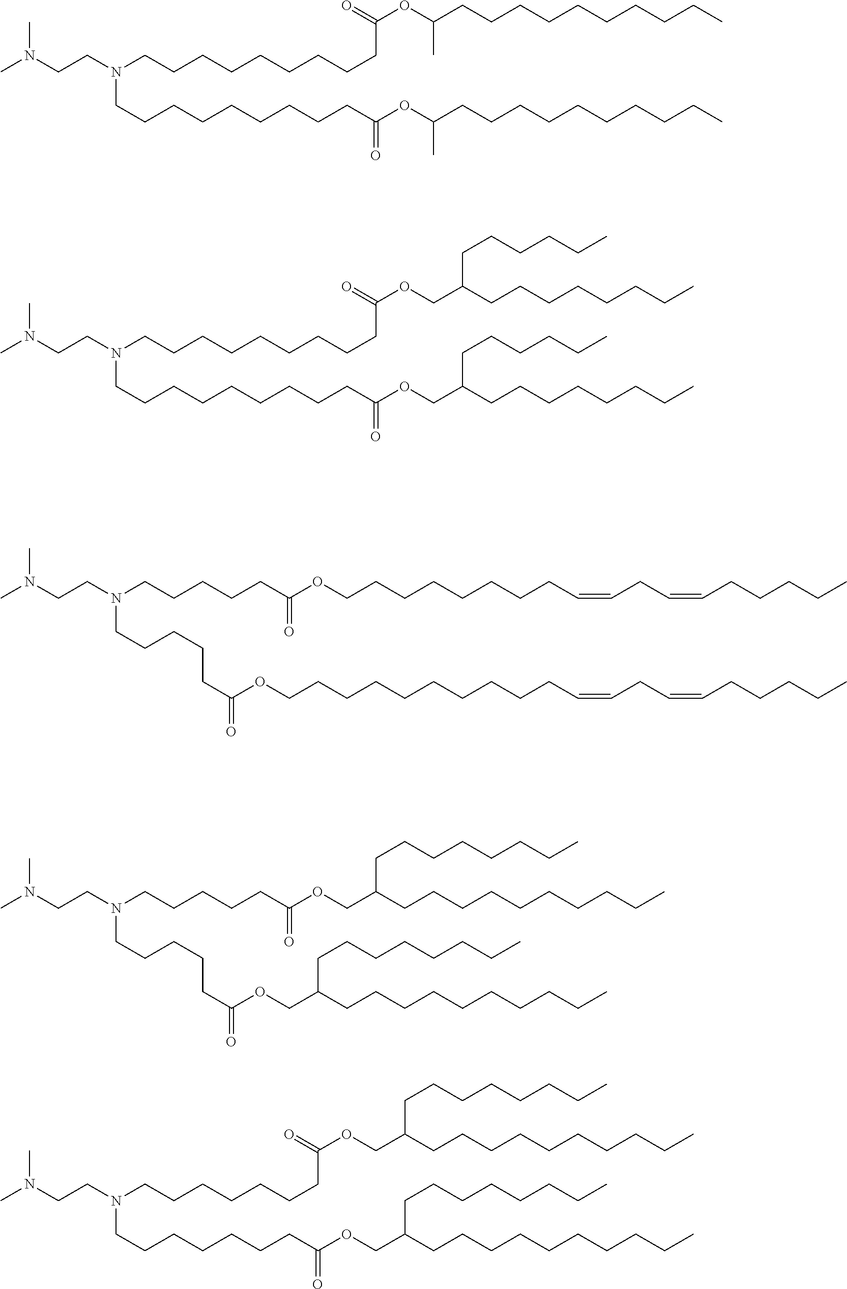

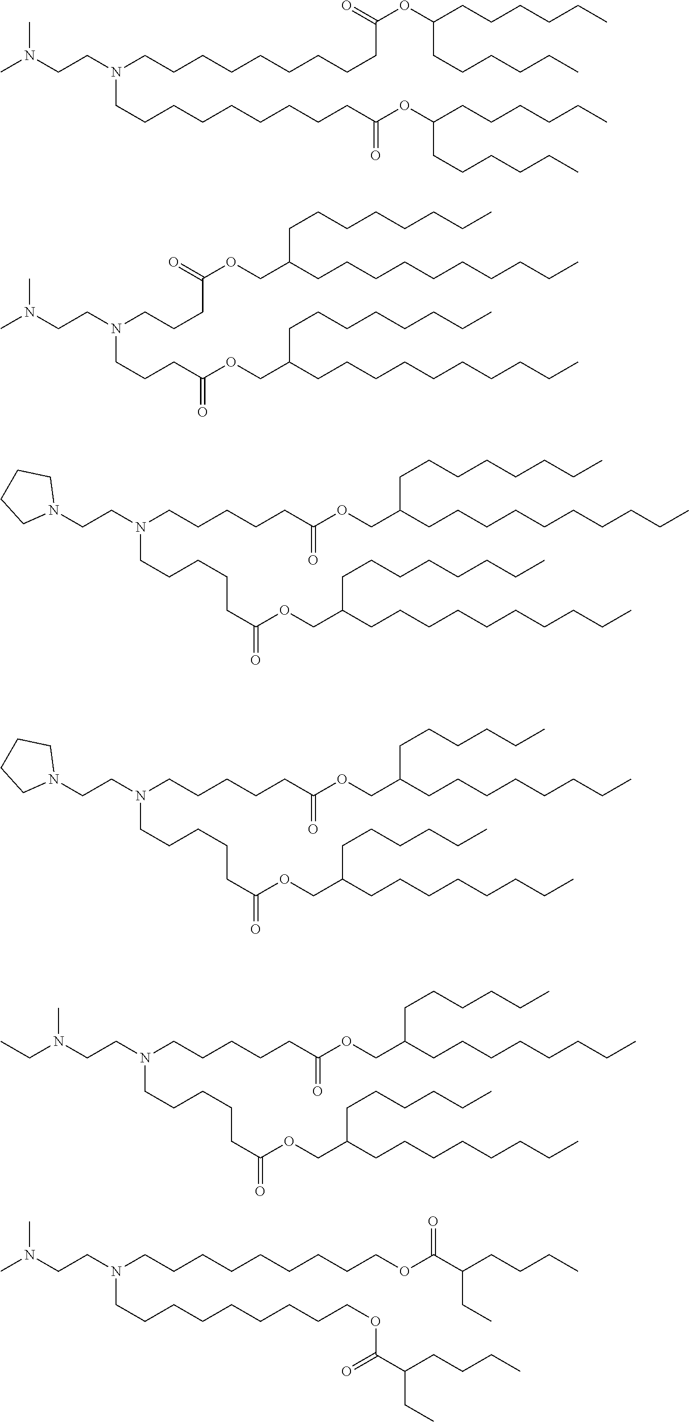

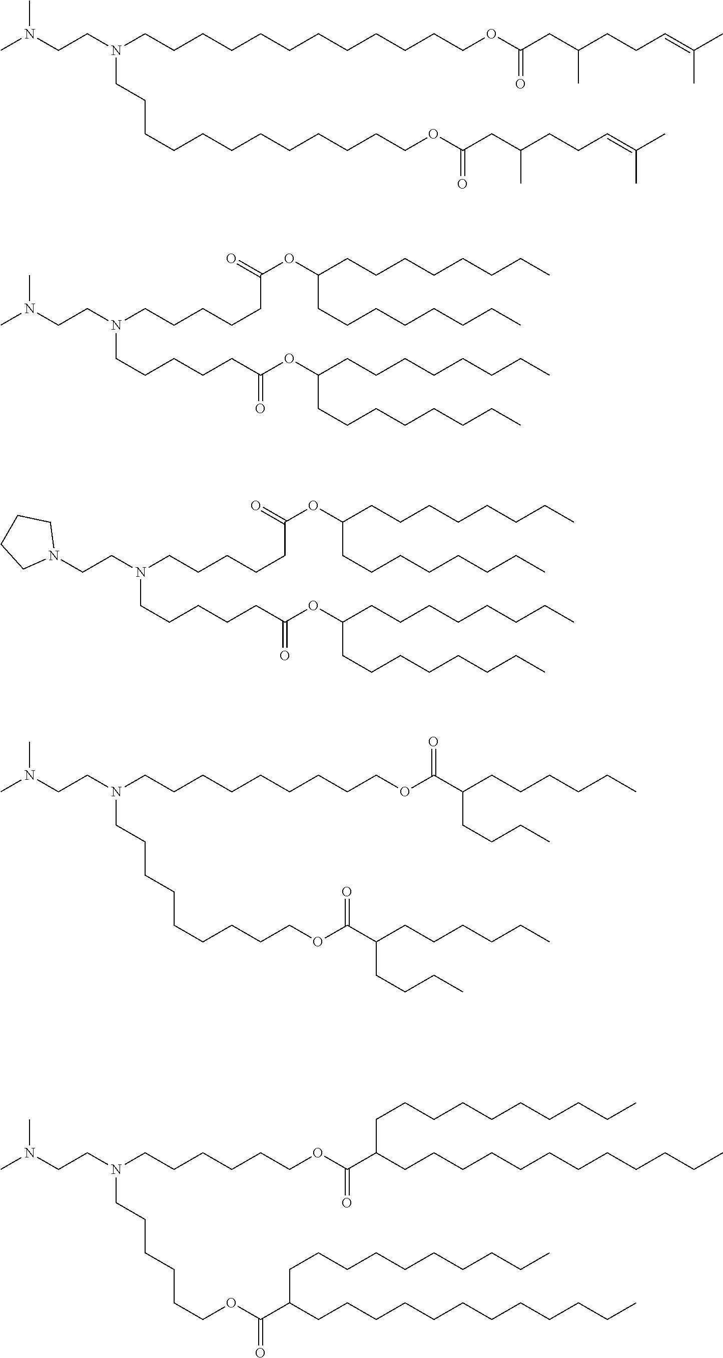

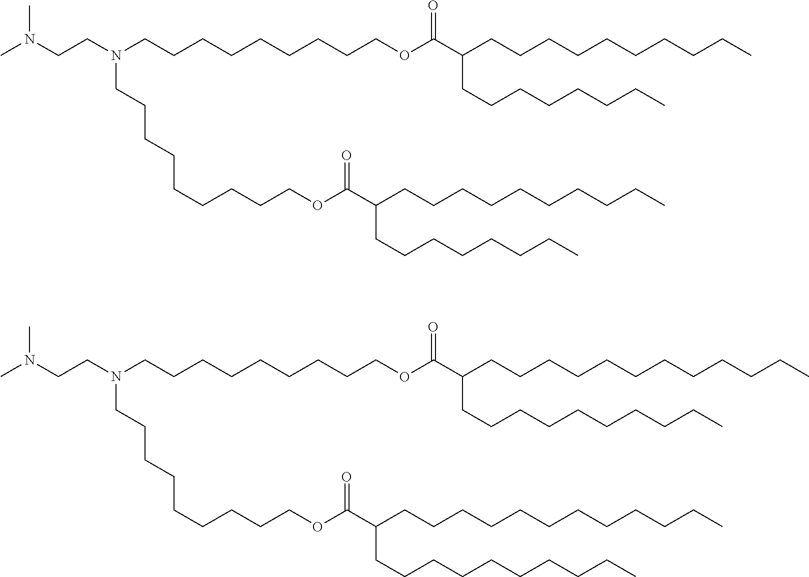

In some embodiments, the LNPs comprise a lipid of Formula (I), at least one agent, and one or more excipients selected from neutral lipids, steroids and pegylated lipids. In some embodiments the lipid of Formula (I) is compound I-5. In some embodiments the lipid of Formula (I) is compound I-6.

In some other embodiments, the cationic lipid component of the LNPs has the structure of Formula (II):

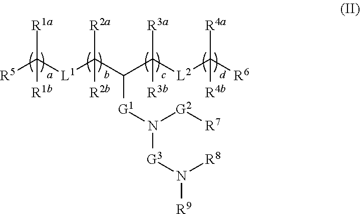

or a pharmaceutically acceptable salt, tautomer, prodrug or stereoisomer thereof, wherein:
L¹ and L² are each independently —O(C═O)—, —(C═O)O—, —C(═O)—, —O—, —S(O)ₓ—, —S—S—, —C(═O)S—, —SC(═O)—, —NR^{a}C(═O)—, —C(═O)NR^{a}—, —NR^{a}C(═O)NR^{a}, —OC(═O)NR^{a}—, —NR^{a}C(═O)O—, or a direct bond;
G¹ is C₁-C₂ alkylene, —(C═O)—, —O(C═O)—, —SC(═O)—, —NR^{a}C(═O)— or a direct bond;
G² is —C(═O)—, —(C—O)O—, —C(═O)S—, —C(═O)NR^{a} or a direct bond;
G³ is C₁-C₆ alkylene;
R^{a} is H or C₁-C₁₂ alkyl;
R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either: (a) H or C₁-C₁₂ alkyl; or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently H or methyl;
R⁷ is C₄-C₂₀ alkyl;
R⁸ and R⁹ are each independently C₁-C₁₂ alkyl; or R⁸ and R⁹, together with the nitrogen atom to which they are attached, form a 5, 6 or 7-membered heterocyclic ring;
a, b, c and d are each independently an integer from 1 to 24; and
x is 0, 1 or 2.

In some embodiments of Formula (II), L¹ and L² are each independently —O(C═O)—, —(C—O)O— or a direct bond. In other embodiments, G¹ and G² are each independently —(C—O)— or a direct bond. In some different embodiments, L¹ and L² are each independently —O(C—O)—, —(C═O)O— or a direct bond; and G¹ and G² are each independently —(C═O)— or a direct bond.

In some different embodiments of Formula (II), L¹ and L² are each independently —C(═O)—, —O—, —S(O)—, —S—S—, —C(═O)S—, —SC(═O)—, —NR^{a}—, —NRC(═O)—, —C(═O)NR^{a}—, —NR^{a}C(═O)NR^{a}, —OC(═O)NR^{a}—, —NR^{a}C(═O)O—, —NR^{a}S(O)ₓNR^{a}—, —NR^{a}S(O)ₓ— or —S(O) «NR^{a}—.

In other of the foregoing embodiments of Formula (II), the lipid compound has one of the following structures (IIA) or (IIB):

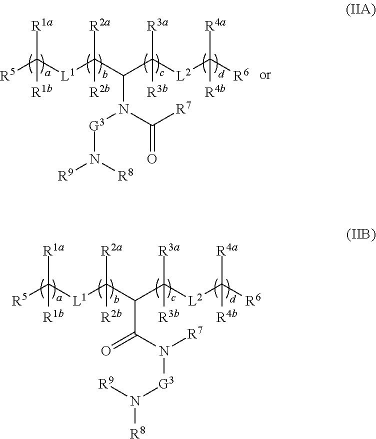

In some embodiments of Formula (II), the lipid compound has structure (IIA). In other embodiments, the lipid compound has structure (IIB).

In any of the foregoing embodiments of Formula (II), one of L¹ or L² is —O(C═O)—. For example, in some embodiments each of L¹ and L² are —O(C═O)—.

In some different embodiments of Formula (II), one of L¹ or L² is —(C═O)O—. For example, in some embodiments each of L¹ and L² is —(C═O)O—.

In different embodiments of Formula (II), one of L¹ or L² is a direct bond. As used herein, a “direct bond” means the group (e.g., L¹ or L²) is absent. For example, in some embodiments each of L¹ and L² is a direct bond.

In other different embodiments of Formula (II), for at least one occurrence of R^{1a} and R^{1b}, R^{1a} is H or C₁-C₁₂ alkyl, and R^{1b} together with the carbon atom to which it is bound is taken together with an adjacent R^{1b} and the carbon atom to which it is bound to form a carbon-carbon double bond.

In still other different embodiments of Formula (II), for at least one occurrence of R^{4a} and R^{4b}, R^{4a} is H or C₁-C₁₂ alkyl, and R^{4b} together with the carbon atom to which it is bound is taken together with an adjacent R^{4b} and the carbon atom to which it is bound to form a carbon-carbon double bond.

In more embodiments of Formula (II), for at least one occurrence of R^{2a} and R^{2b}, R^{2a} is H or C₁-C₁₂ alkyl, and R^{2b} together with the carbon atom to which it is bound is taken together with an adjacent R^{2b} and the carbon atom to which it is bound to form a carbon-carbon double bond.

In other different embodiments of Formula (II), for at least one occurrence of R^{3a} and R^{3b}, R^{3a} is H or C₁-C₁₂ alkyl, and R^{3b} together with the carbon atom to which it is bound is taken together with an adjacent R^{3b} and the carbon atom to which it is bound to form a carbon-carbon double bond.

In various other embodiments of Formula (II), the lipid compound has one of the following structures (IIC) or (IID):

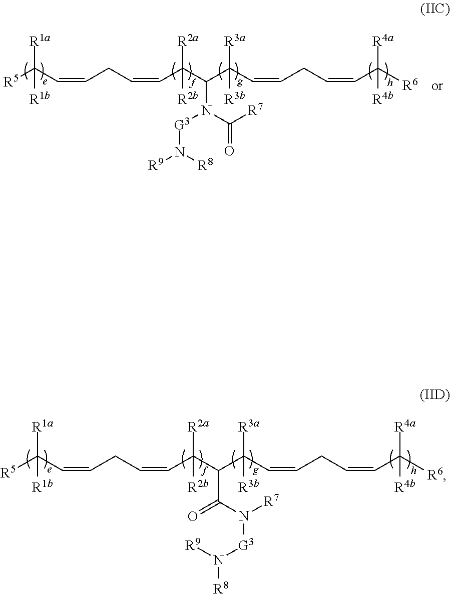

wherein e, f, g and h are each independently an integer from 1 to 12.

In some embodiments of Formula (II), the lipid compound has structure (IIC). In other embodiments, the lipid compound has structure (IID).

In various embodiments of structures (IIC) or (IID), e, f, g and h are each independently an integer from 4 to 10.

In certain embodiments of Formula (II), a, b, c and d are each independently an integer from 2 to 12 or an integer from 4 to 12. In other embodiments, a, b, c and d are each independently an integer from 8 to 12 or 5 to 9. In some certain embodiments, a is 0. In some embodiments, a is 1. In other embodiments, a is 2. In more embodiments, a is 3. In yet other embodiments, a is 4. In some embodiments, a is 5. In other embodiments, a is 6. In more embodiments, a is 7. In yet other embodiments, a is 8. In some embodiments, a is 9. In other embodiments, a is 10. In more embodiments, a is 11. In yet other embodiments, a is 12. In some embodiments, a is 13. In other embodiments, a is 14. In more embodiments, a is 15. In yet other embodiments, a is 16.

In some embodiments of Formula (II), b is 1. In other embodiments, b is 2. In more embodiments, b is 3. In yet other embodiments, b is 4. In some embodiments, b is 5. In other embodiments, b is 6. In more embodiments, b is 7. In yet other embodiments, b is 8. In some embodiments, b is 9. In other embodiments, b is 10. In more embodiments, b is 11. In yet other embodiments, b is 12. In some embodiments, b is 13. In other embodiments, b is 14. In more embodiments, b is 15. In yet other embodiments, b is 16.

In some embodiments of Formula (II), c is 1. In other embodiments, c is 2. In more embodiments, c is 3. In yet other embodiments, c is 4. In some embodiments, c is 5. In other embodiments, c is 6. In more embodiments, c is 7. In yet other embodiments, c is 8. In some embodiments, c is 9. In other embodiments, c is 10. In more embodiments, c is 11. In yet other embodiments, c is 12. In some embodiments, c is 13. In other embodiments, c is 14. In more embodiments, c is 15. In yet other embodiments, c is 16.

In some certain embodiments of Formula (II), d is 0. In some embodiments, d is 1. In other embodiments, dis 2. In more embodiments, d is 3. In yet other embodiments, dis 4. In some embodiments, d is 5. In other embodiments, d is 6. In more embodiments, dis 7. In yet other embodiments, d is 8. In some embodiments, d is 9. In other embodiments, dis 10. In more embodiments, dis 11. In yet other embodiments, dis 12. In some embodiments, dis 13. In other embodiments, dis 14. In more embodiments, dis 15. In yet other embodiments, dis 16.

In some embodiments of Formula (II), e is 1. In other embodiments, e is 2. In more embodiments, e is 3. In yet other embodiments, e is 4. In some embodiments, e is 5. In other embodiments, e is 6. In more embodiments, e is 7. In yet other embodiments, e is 8. In some embodiments, e is 9. In other embodiments, e is 10. In more embodiments, e is 11. In yet other embodiments, e is 12.

In some embodiments of Formula (II), f is 1. In other embodiments, f is 2. In more embodiments, f is 3. In yet other embodiments, f is 4. In some embodiments, f is 5. In other embodiments, f is 6. In more embodiments, f is 7. In yet other embodiments, f is 8. In some embodiments, f is 9. In other embodiments, f is 10. In more embodiments, f is 11. In yet other embodiments, f is 12.

In some embodiments of Formula (II), g is 1. In other embodiments, g is 2. In more embodiments, g is 3. In yet other embodiments, g is 4. In some embodiments, g is 5. In other embodiments, g is 6. In more embodiments, g is 7. In yet other embodiments, g is 8. In some embodiments, g is 9. In other embodiments, g is 10. In more embodiments, g is 11. In yet other embodiments, g is 12.

In some embodiments of Formula (II), h is 1. In other embodiments, e is 2. In more embodiments, h is 3. In yet other embodiments, h is 4. In some embodiments, e is 5. In other embodiments, h is 6. In more embodiments, h is 7. In yet other embodiments, h is 8. In some embodiments, h is 9. In other embodiments, h is 10. In more embodiments, h is 11. In yet other embodiments, h is 12.

In some other various embodiments of Formula (II), a and d are the same. In some other embodiments, b and c are the same. In some other specific embodiments and a and d are the same and b and c are the same.

The sum of a and b and the sum of c and d of Formula (II) are factors which may be varied to obtain a lipid having the desired properties. In one embodiment, a and b are chosen such that their sum is an integer ranging from 14 to 24. In other embodiments, c and d are chosen such that their sum is an integer ranging from 14 to 24. In further embodiment, the sum of a and b and the sum of c and d are the same. For example, in some embodiments the sum of a and b and the sum of c and d are both the same integer which may range from 14 to 24. In still more embodiments, a, b, c and d are selected such that the sum of a and b and the sum of c and dis 12 or greater.

The substituents at R^{1a}, R^{2a}, R^{3a} and R^{4a} of Formula (II) are not particularly limited. In some embodiments, at least one of R^{1a}, R^{2a}, R^{3a} and R^{4a} is H. In certain embodiments R^{1a}, R^{2a}, R^{3a} and R^{4a} are H at each occurrence. In certain other embodiments at least one of R^{1a}, R^{2a}, R^{3a} and R^{4a} is C₁-C₁₂ alkyl. In certain other embodiments at least one of R^{1a}, R^{2a}, R^{3a} and R^{4a} is C₁-C₈ alkyl. In certain other embodiments at least one of R^{1a}, R^{2a}, R^{3a} and R^{4a} is C₁-C₆ alkyl. In some of the foregoing embodiments, the C₁-C₈alkyl is methyl, ethyl, n-propyl, iso-propyl, n-butyl, iso-butyl, tert-butyl, n-hexyl or n-octyl.

In certain embodiments of Formula (II), R^{1a}, R^{1b}, R^{4a} and R^{4b} are C₁-C₁₂ alkyl at each occurrence.

In further embodiments of Formula (II), at least one of R^{1b}, R^{2b}, R^{3b} and R^{4b} is H or R^{1b}, R^{2b}, R^{3b} and R^{4b} are H at each occurrence.

In certain embodiments of Formula (II), R^{1b} together with the carbon atom to which it is bound is taken together with an adjacent R^{1b} and the carbon atom to which it is bound to form a carbon-carbon double bond. In other embodiments of the foregoing R^{4b} together with the carbon atom to which it is bound is taken together with an adjacent R^{4b} and the carbon atom to which it is bound to form a carbon-carbon double bond.

The substituents at R⁵ and R⁶ of Formula (II) are not particularly limited in the foregoing embodiments. In certain embodiments one of R⁵ or R⁶ is methyl. In other embodiments each of R⁵ or R⁶ is methyl.

The substituents at R⁷ of Formula (II) are not particularly limited in the foregoing embodiments. In certain embodiments R⁷ is C₆-C₁₆ alkyl. In some other embodiments, R⁷ is C₆-C₉ alkyl. In some of these embodiments, R⁷ is substituted with —(C═O)OR^{b}, —O(C═O)R^{b}, —C(═O)R^{b}, —OR^{b}, —S(O)ₓR^{b}, —S—SR^{b}, —C(═O)SR^{b}, —SC(═O)R^{b}, —NR^{a}R^{b}, —NR^{a}C(═O)R^{b}, —C(═O)NR^{a}R^{b}, —NR^{a}C(═O)NR^{a}R^{b}, —OC(═O)NR^{a}R^{b}, —NR^{a}C(═O)OR^{b}, —NR^{a}S(O) \NRaRb, —NR^{a}S(O)ₓR^{b} or —S(O)ₓNR^{a}R^{b}, wherein: R^{a} is H or C₁-C₁₂ alkyl; Rbis C₁-C₁₅ alkyl; and x is 0, 1 or 2. For example, in some embodiments R⁷ is substituted with —(C═O)OR^{b} or —O(C═O)R^{b}.

In various of the foregoing embodiments of Formula (II), R^{b} is branched C₁-C₁₅ alkyl. For example, in some embodiments RD has one of the following structures:

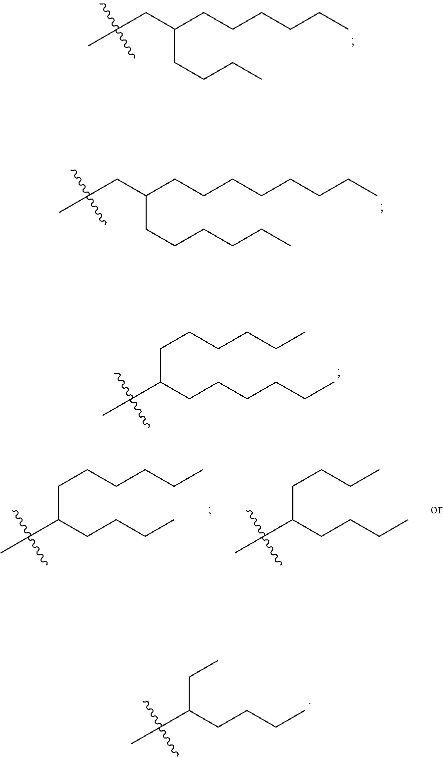

In certain other of the foregoing embodiments of Formula (II), one of R⁸ or R⁹ is methyl. In other embodiments, both R⁸ and R⁹ are methyl.

In some different embodiments of Formula (II), R⁸ and R⁹, together with the nitrogen atom to which they are attached, form a 5, 6 or 7-membered heterocyclic ring. In some embodiments of the foregoing, R⁸ and R⁹, together with the nitrogen atom to which they are attached, form a 5-membered heterocyclic ring, for example a pyrrolidinyl ring. In some different embodiments of the foregoing, R⁸ and R⁹, together with the nitrogen atom to which they are attached, form a 6-membered heterocyclic ring, for example a piperazinyl ring.

In still other embodiments of the foregoing lipids of Formula (II), G³ is C₂-C₄ alkylene, for example C₃ alkylene.

In various different embodiments, the lipid compound has one of the following structures:

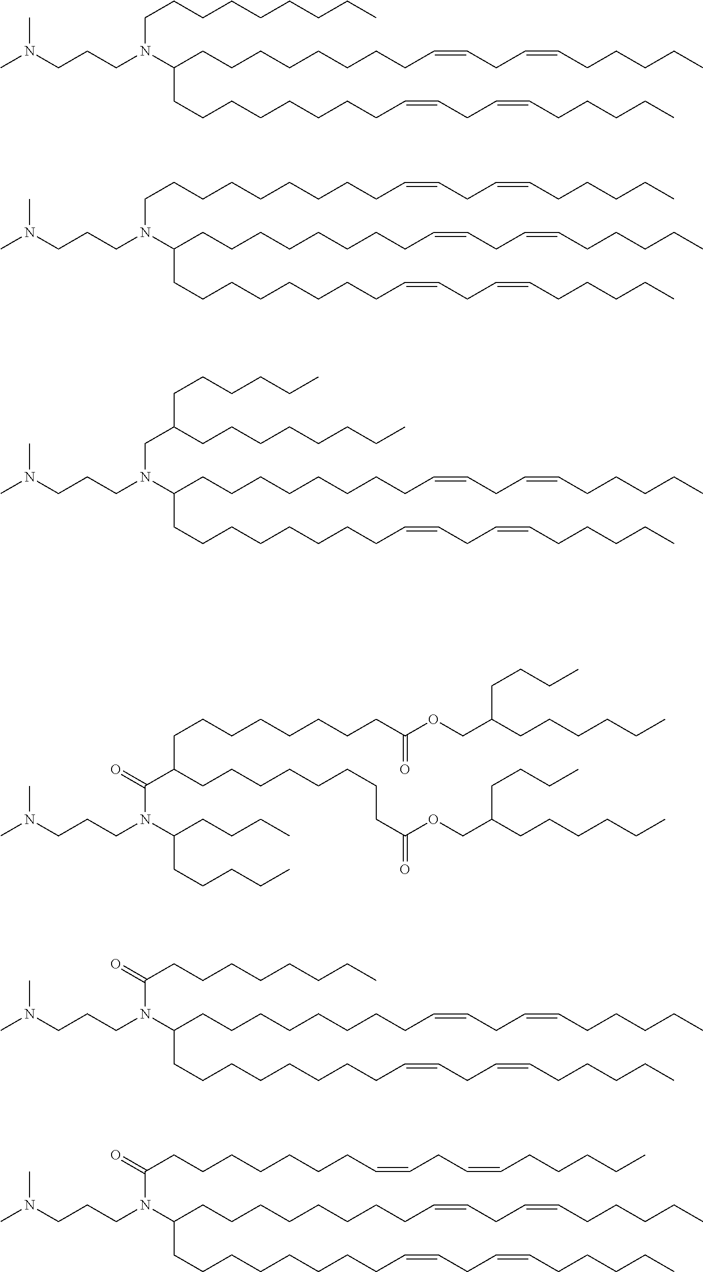

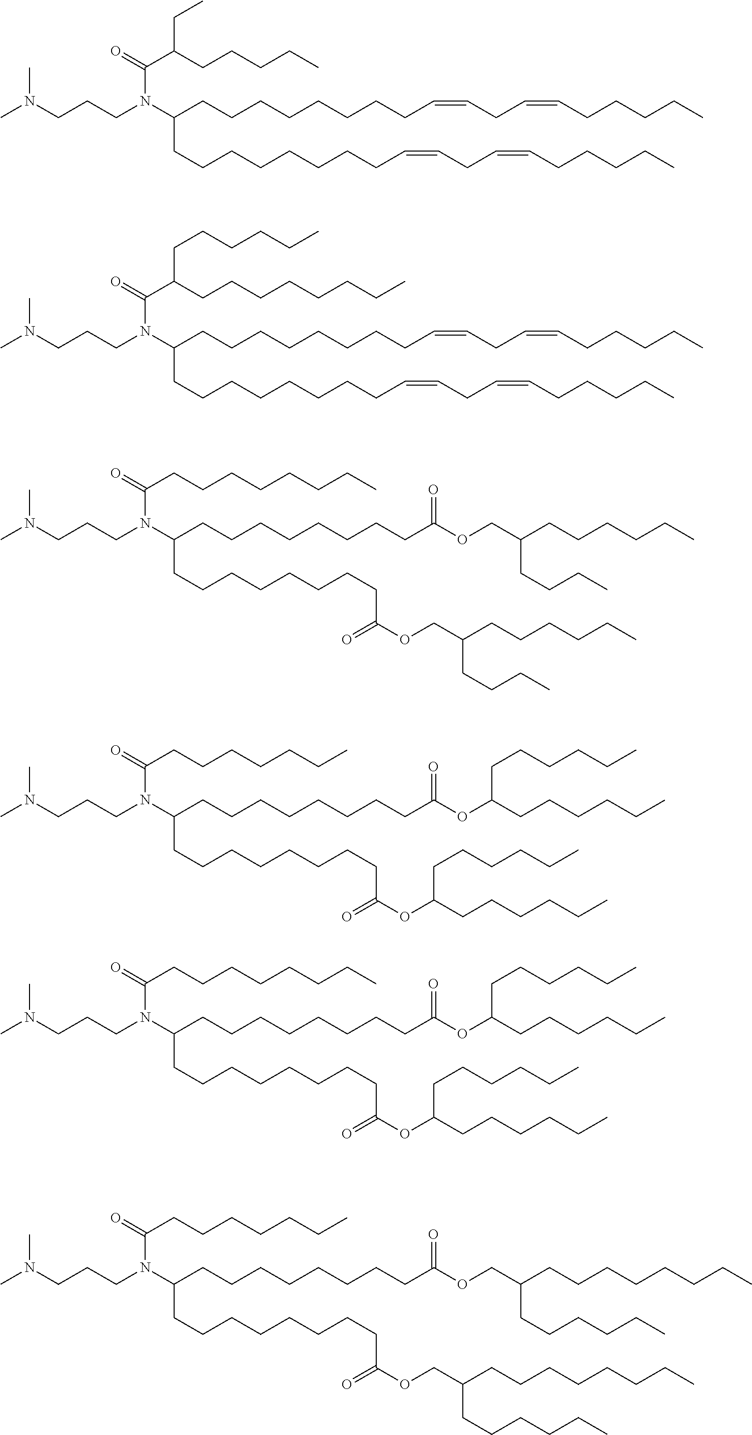

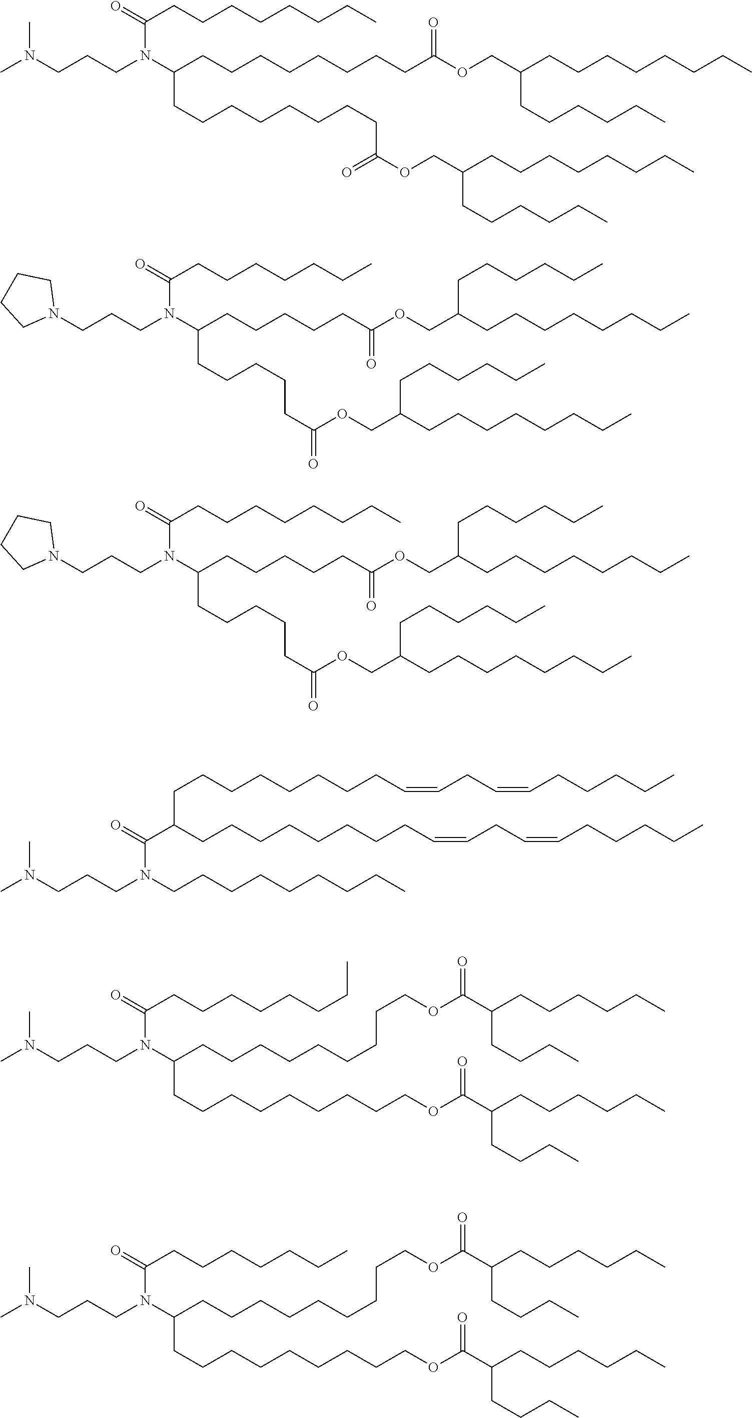

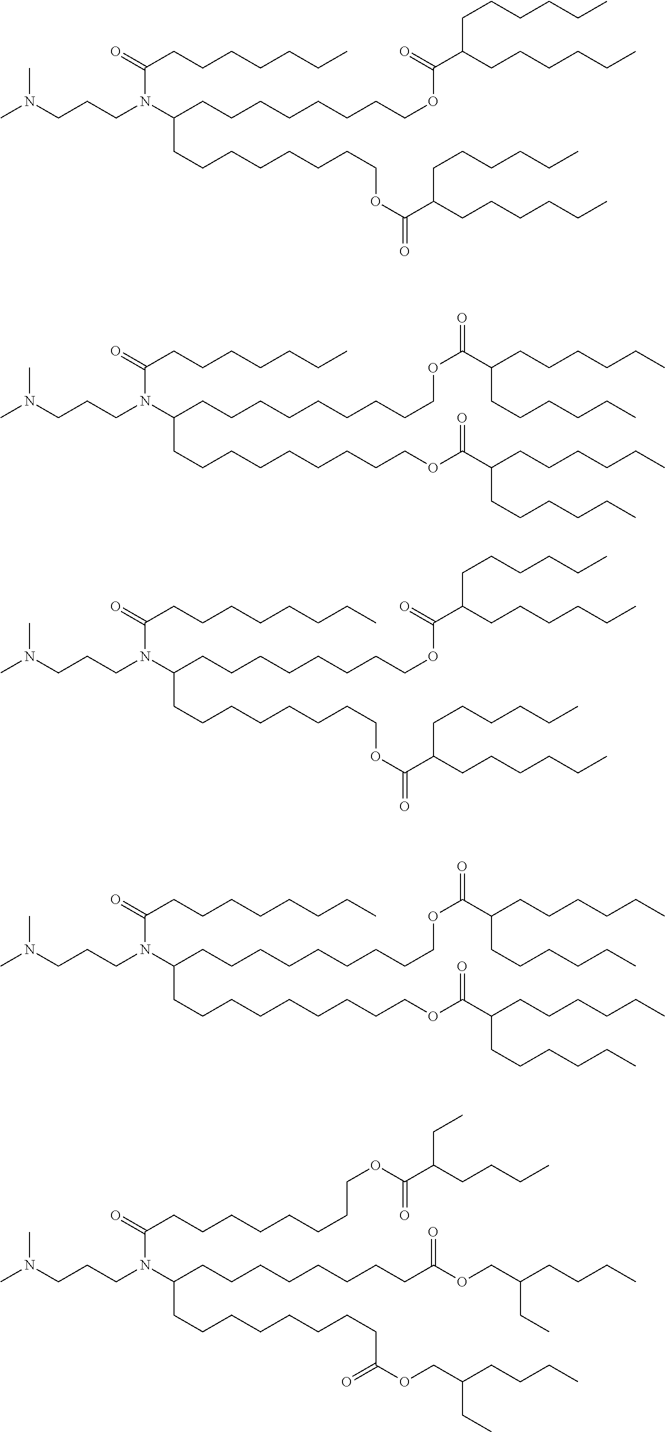

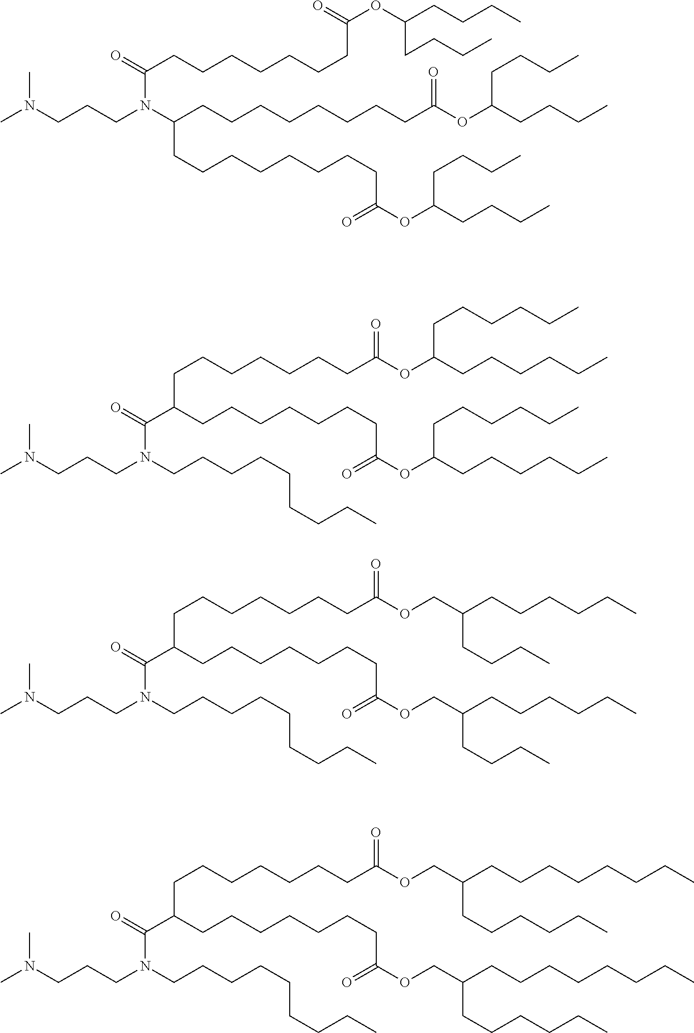

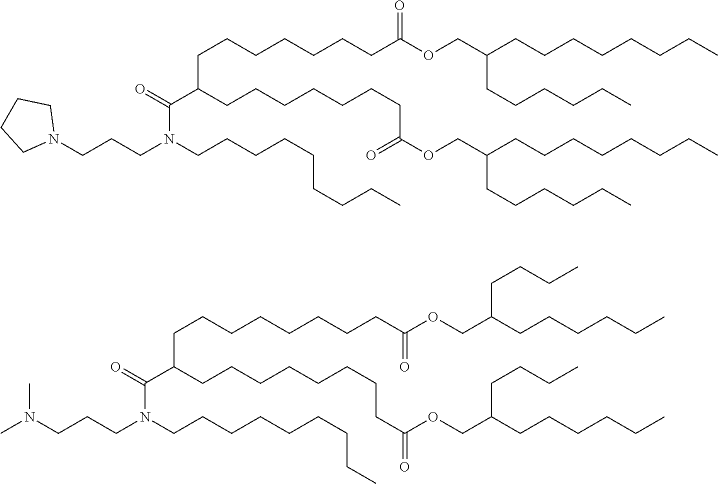

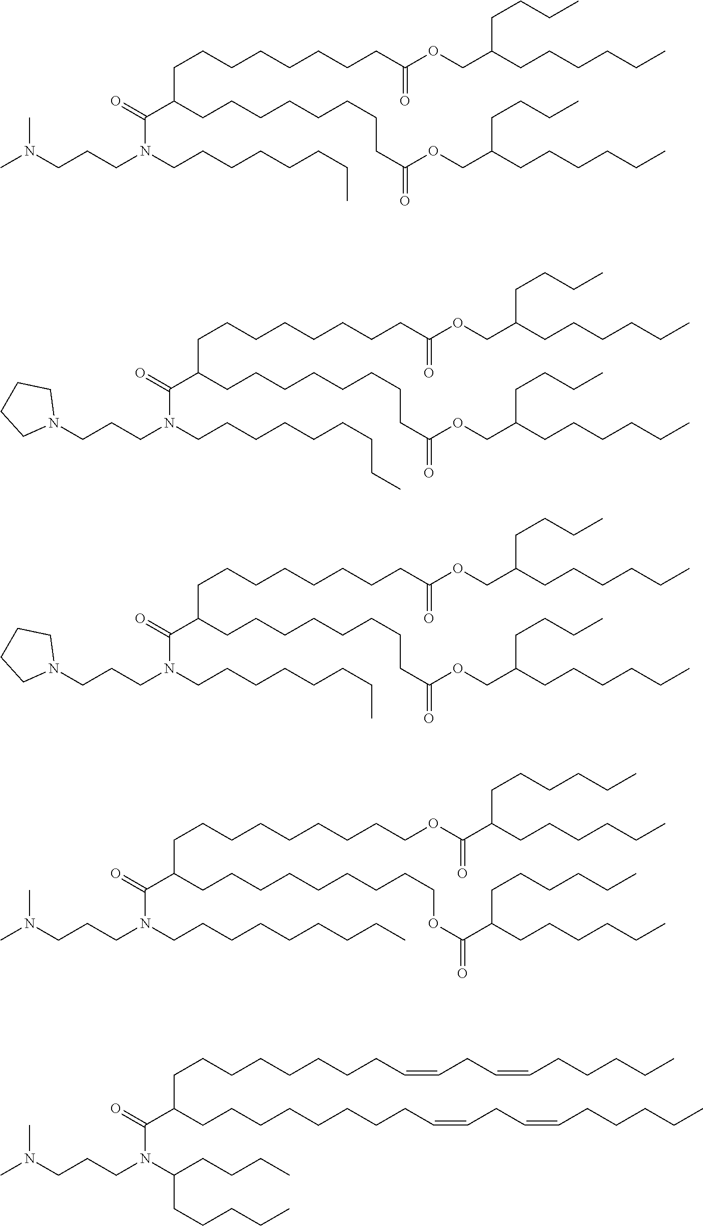

In some embodiments, the LNPs comprise a lipid of Formula (II), at least one agent, and one or more excipient selected from neutral lipids, steroids and pegylated lipids. In some embodiments, the lipid of Formula (II) is compound II-9. In some embodiments, the lipid of Formula (II) is compound II-10. In some embodiments, the lipid of Formula (II) is compound II-11. In some embodiments, the lipid of Formula (II) is compound II-12. In some embodiments, the lipid of Formula (II) is compound II-32.

In some other embodiments, the cationic lipid component of the LNPs has the structure of Formula (III):

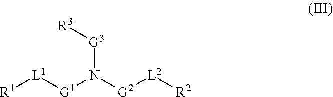

or a pharmaceutically acceptable salt, tautomer, prodrug or stereoisomer thereof, wherein:
one of L¹ or L² is —O(C═O)—, —(C—O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O—, and the other of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR³, CN, —C(═O) OR⁴, —OC(═O)R⁴ or —NR⁵C(═O)R⁴;
R⁴ is C₁-C₁₂ alkyl;
R⁵ is H or C₁-C₆ alkyl; and
x is 0, 1 or 2.

In some of the foregoing embodiments of Formula (III), the lipid has one of the following structures (IIIA) or (IIIB):

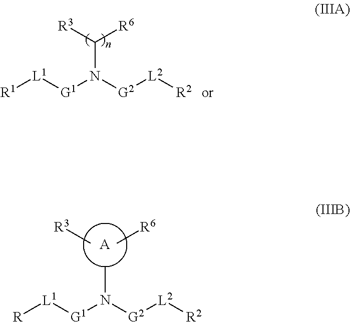

wherein:
A is a 3 to 8-membered cycloalkyl or cycloalkylene ring;
R⁶ is, at each occurrence, independently H, OH or C₁-C₂₄ alkyl;
n is an integer ranging from 1 to 15.

In some of the foregoing embodiments of Formula (III), the lipid has structure (IIIA), and in other embodiments, the lipid has structure (IIIB).

In other embodiments of Formula (III), the lipid has one of the following structures (IIIC) or (IIID):

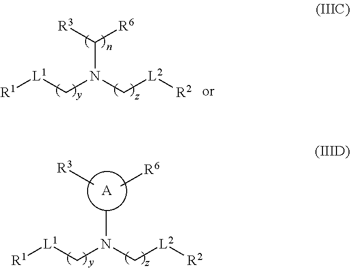

wherein y and z are each independently integers ranging from 1 to 12.

In any of the foregoing embodiments of Formula (III), one of L¹ or L² is —O(C═O)—. For example, in some embodiments each of L¹ and L² are —O(C═O)—. In some different embodiments of any of the foregoing, L¹ and L² are each independently —(C═O)O— or —O(C═O)—. For example, in some embodiments each of L¹ and L² is —(C═O)O—.

In some different embodiments of Formula (III), the lipid has one of the following structures (IIIE) or (IIIF):

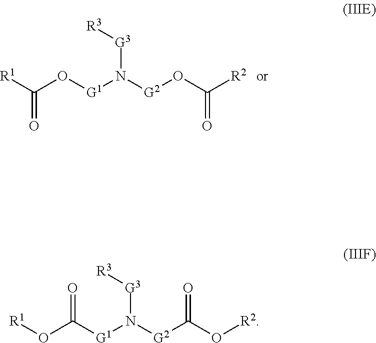

In some of the foregoing embodiments of Formula (III), the lipid has one of the following structures (IIIG), (IIIH), (IIII), or (IIIJ):

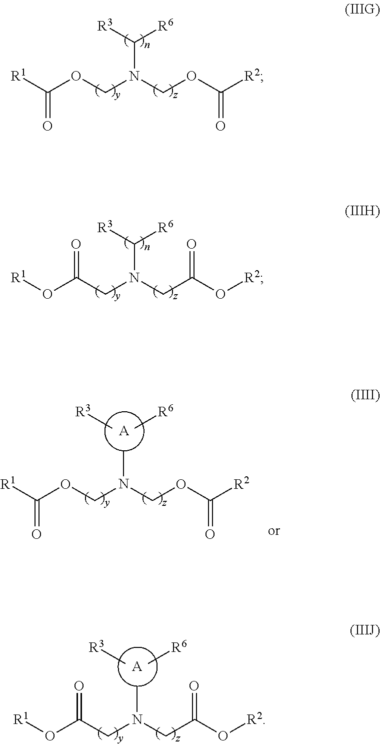

In some of the foregoing embodiments of Formula (III), n is an integer ranging from 2 to 12, for example from 2 to 8 or from 2 to 4. For example, in some embodiments, n is 3, 4, 5 or 6. In some embodiments, n is 3. In some embodiments, n is 4. In some embodiments, n is 5. In some embodiments, n is 6.

In some other of the foregoing embodiments of Formula (III), y and z are each independently an integer ranging from 2 to 10. For example, in some embodiments, y and z are each independently an integer ranging from 4 to 9 or from 4 to 6.

In some of the foregoing embodiments of Formula (III), R⁶ is H. In other of the foregoing embodiments, R⁶ is C₁-C₂₄ alkyl. In other embodiments, R⁶ is OH.

In some embodiments of Formula (III), G³ is unsubstituted. In other embodiments, G³ is substituted. In various different embodiments, G³ is linear C₁-C₂₄ alkylene or linear C₁-C₂₄ alkenylene.

In some other foregoing embodiments of Formula (III), R¹ or R², or both, is C₆-C₂₄ alkenyl. For example, in some embodiments, R¹ and R² each, independently have the following structure:

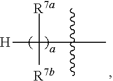

wherein:
R^{7a} and R^{7b} are, at each occurrence, independently H or C₁-C₁₂ alkyl; and
a is an integer from 2 to 12,
   wherein R^{7a}, R^{7b} and a are each selected such that R¹ and R² each independently comprise from 6 to 20 carbon atoms. For example, in some embodiments a is an integer ranging from 5 to 9 or from 8 to 12.

In some of the foregoing embodiments of Formula (III), at least one occurrence of R^{7a} is H. For example, in some embodiments, R^{7a} is H at each occurrence. In other different embodiments of the foregoing, at least one occurrence of R^{7b} is C₁-C₈ alkyl. For example, in some embodiments, C₁-C₈alkyl is methyl, ethyl, n-propyl, iso-propyl, n-butyl, iso-butyl, tert-butyl, n-hexyl or n-octyl.

In different embodiments of Formula (III), R¹ or R², or both, has one of the following structures:

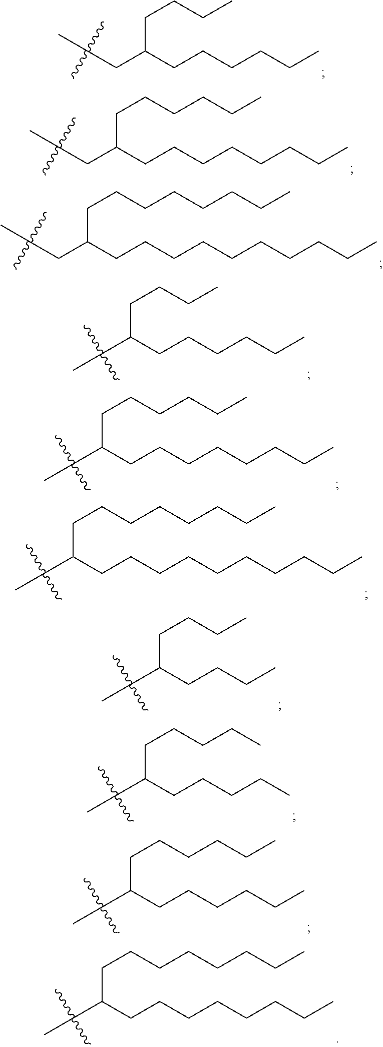

In some of the foregoing embodiments of Formula (III), R³ is OH, CN, —C(═O)OR⁴, —OC(═O)R⁴ or —NHC(═O)R⁴. In some embodiments, R⁴ is methyl or ethyl.

In various different embodiments, the cationic lipid of Formula (III) has one of the following structures:

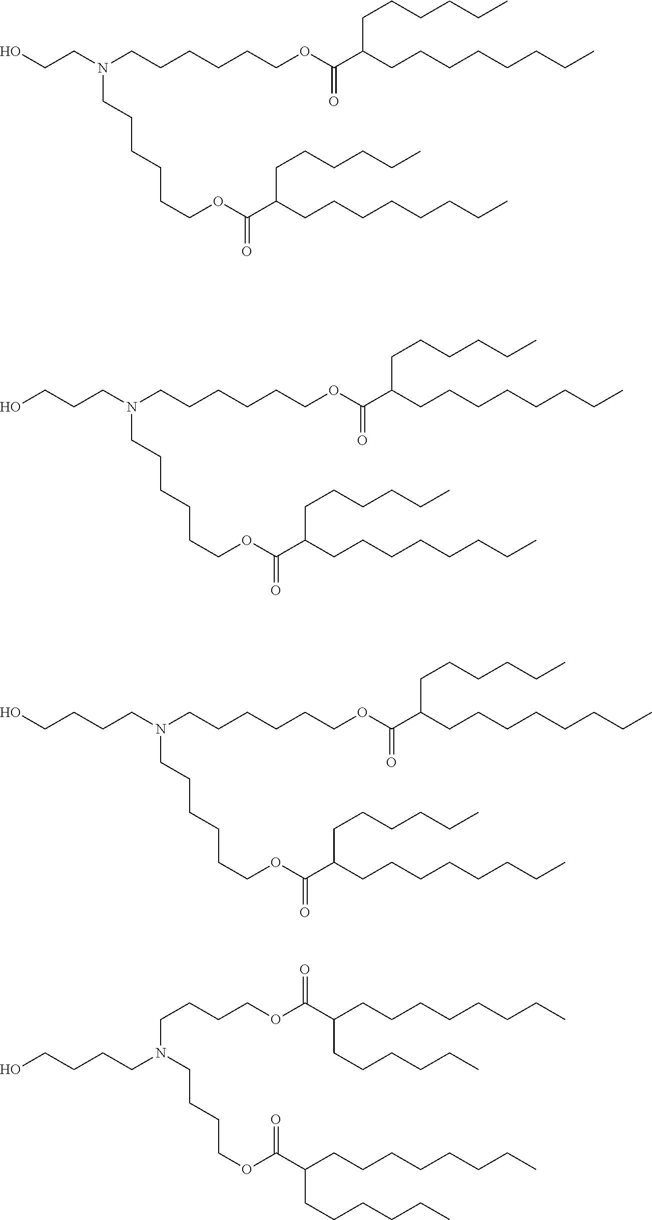 [SMILES: CCCCCCCCC(CCCCCC)C(=O)OCCCCCCN(CCO)CCCCCCOC(=O)C(CCCCCC)CCCCCCCC ; CCCCCCCCC(CCCCCC)C(=O)OCCCCCCN(CCCO)CCCCCCOC(=O)C(CCCCCC)CCCCCCCC ; CCCCCCCCC(CCCCCC)C(=O)OCCCCCCN(CCCCO)CCCCCCOC(=O)C(CCCCCC)CCCCCCCC ; CCCCCCCCC(CCCCCC)C(=O)OCCCCN(CCCCO)CCCCOC(=O)C(CCCCCC)CCCCCCCC]

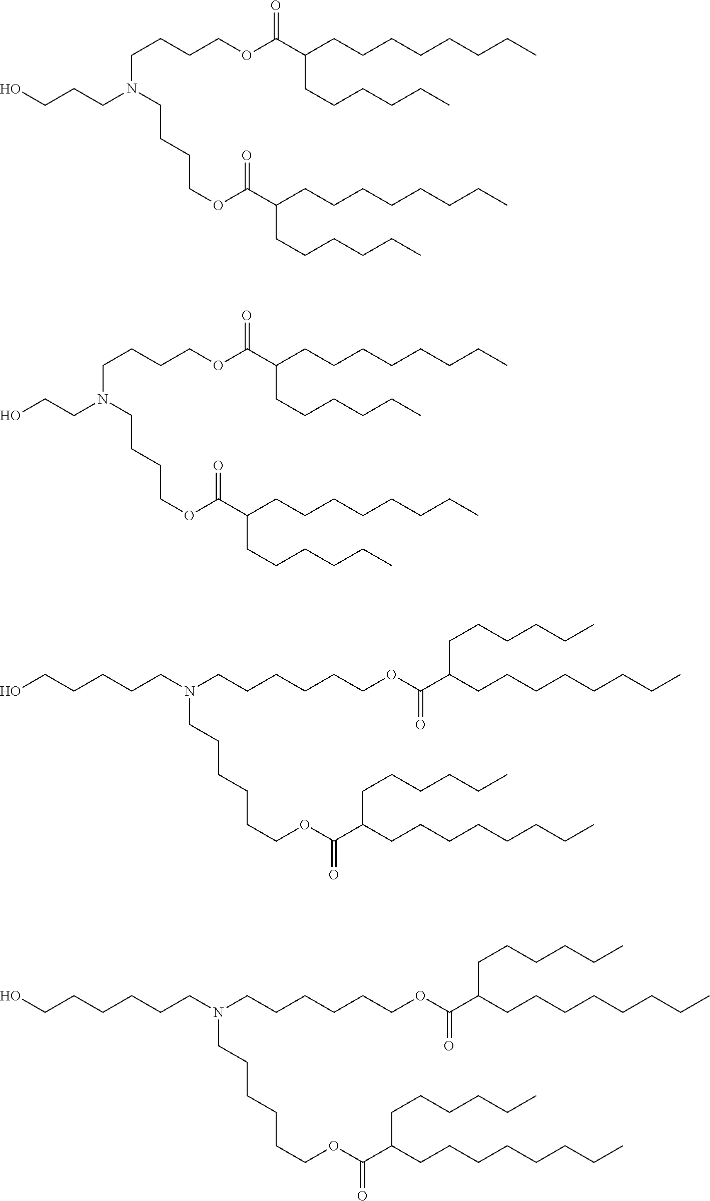 [SMILES: CCCCCCCCC(CCCCCC)C(=O)OCCCCN(CCCO)CCCCOC(=O)C(CCCCCC)CCCCCCCC ; CCCCCCCCC(CCCCCC)C(=O)OCCCCN(CCO)CCCCOC(=O)C(CCCCCC)CCCCCCCC ; CCCCCCCCC(CCCCCC)C(=O)OCCCCCCN(CCCCCO)CCCCCCOC(=O)C(CCCCCC)CCCCCCCC ; CCCCCCCCC(CCCCCC)C(=O)OCCCCCCN(CCCCCCO)CCCCCCOC(=O)C(CCCCCC)CCCCCCCC]

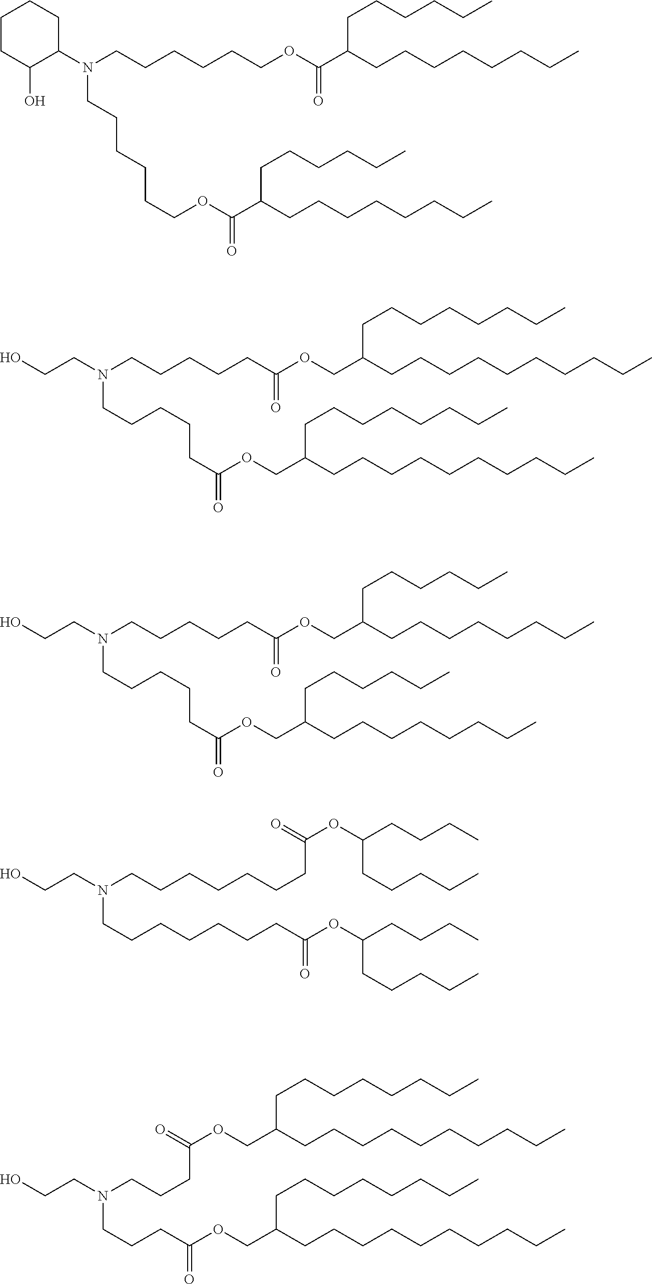 [SMILES: CCCCCCCCC(CCCCCC)C(=O)OCCCCCCN(CCCCCCOC(=O)C(CCCCCC)CCCCCCCC)C1CCCCC1O ; CCCCCCCCCCC(CCCCCCCC)COC(=O)CCCCCN(CCO)CCCCCC(=O)OCC(CCCCCCCC)CCCCCCCCCC ; CCCCCCCCC(CCCCCC)COC(=O)CCCCCN(CCO)CCCCCC(=O)OCC(CCCCCC)CCCCCCCC ; CCCCCC(CCCC)OC(=O)CCCCCCCN(CCO)CCCCCCCC(=O)OC(CCCC)CCCCC ; CCCCCCCCCCC(CCCCCCCC)COC(=O)CCCN(CCO)CCCC(=O)OCC(CCCCCCCC)CCCCCCCCCC]

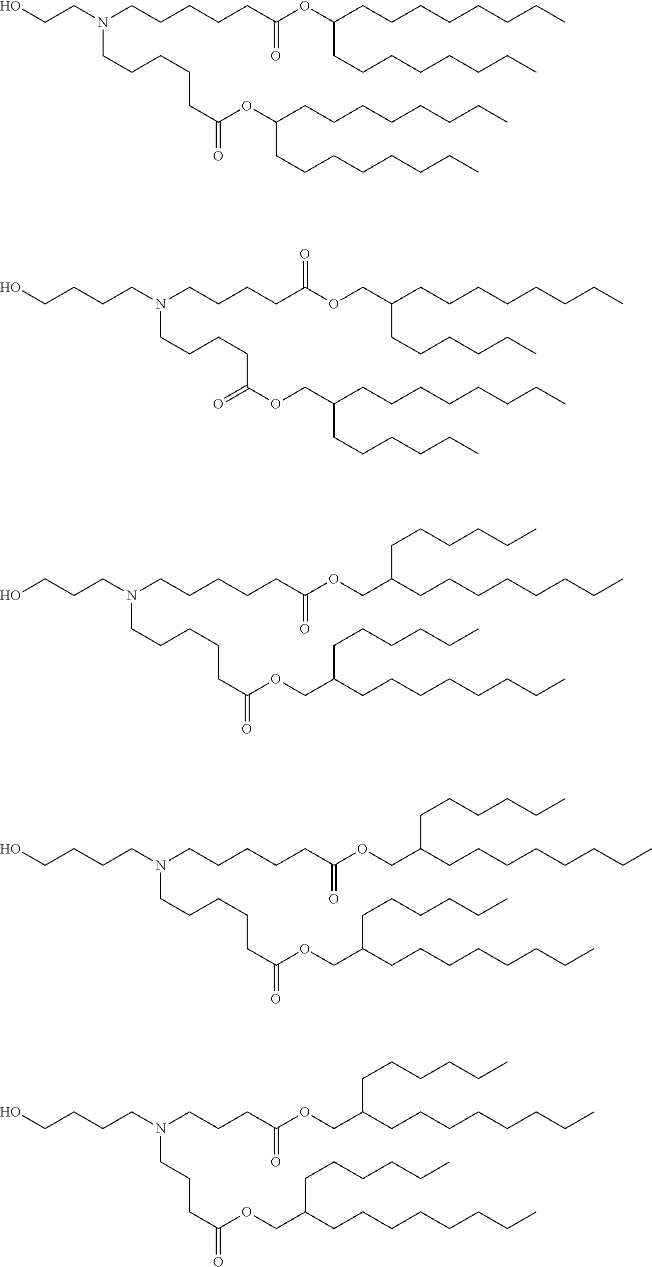 [SMILES: CCCCCCCCC(CCCCCCCC)OC(=O)CCCCCN(CCO)CCCCCC(=O)OC(CCCCCCCC)CCCCCCCC ; CCCCCCCCC(CCCCCC)COC(=O)CCCCN(CCCCO)CCCCC(=O)OCC(CCCCCC)CCCCCCCC ; CCCCCCCCC(CCCCCC)COC(=O)CCCCCN(CCCO)CCCCCC(=O)OCC(CCCCCC)CCCCCCCC ; CCCCCCCCC(CCCCCC)COC(=O)CCCCCN(CCCCO)CCCCCC(=O)OCC(CCCCCC)CCCCCCCC ; CCCCCCCCC(CCCCCC)COC(=O)CCCN(CCCCO)CCCC(=O)OCC(CCCCCC)CCCCCCCC]

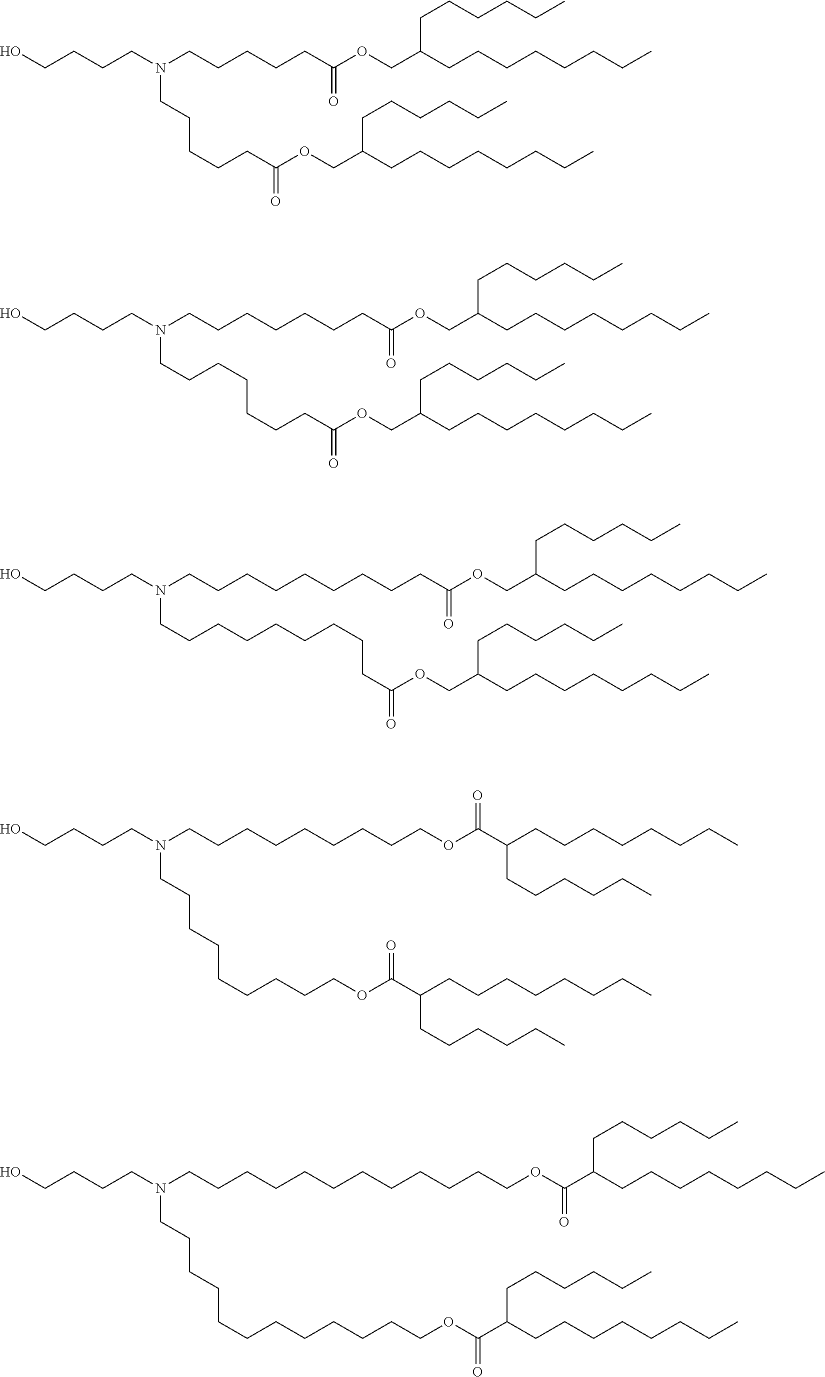 [SMILES: CCCCCCCCC(CCCCCC)COC(=O)CCCCCN(CCCCO)CCCCCC(=O)OCC(CCCCCC)CCCCCCCC ; CCCCCCCCC(CCCCCC)COC(=O)CCCCCCCN(CCCCO)CCCCCCCC(=O)OCC(CCCCCC)CCCCCCCC ; CCCCCCCCC(CCCCCC)COC(=O)CCCCCCCCCN(CCCCO)CCCCCCCCCC(=O)OCC(CCCCCC)CCCCCCCC ; CCCCCCCCC(CCCCCC)C(=O)OCCCCCCCCCN(CCCCO)CCCCCCCCCOC(=O)C(CCCCCC)CCCCCCCC ; CCCCCCCCC(CCCCCC)C(=O)OCCCCCCCCCCCCN(CCCCO)CCCCCCCCCCCCOC(=O)C(CCCCCC)CCCCCCCC]

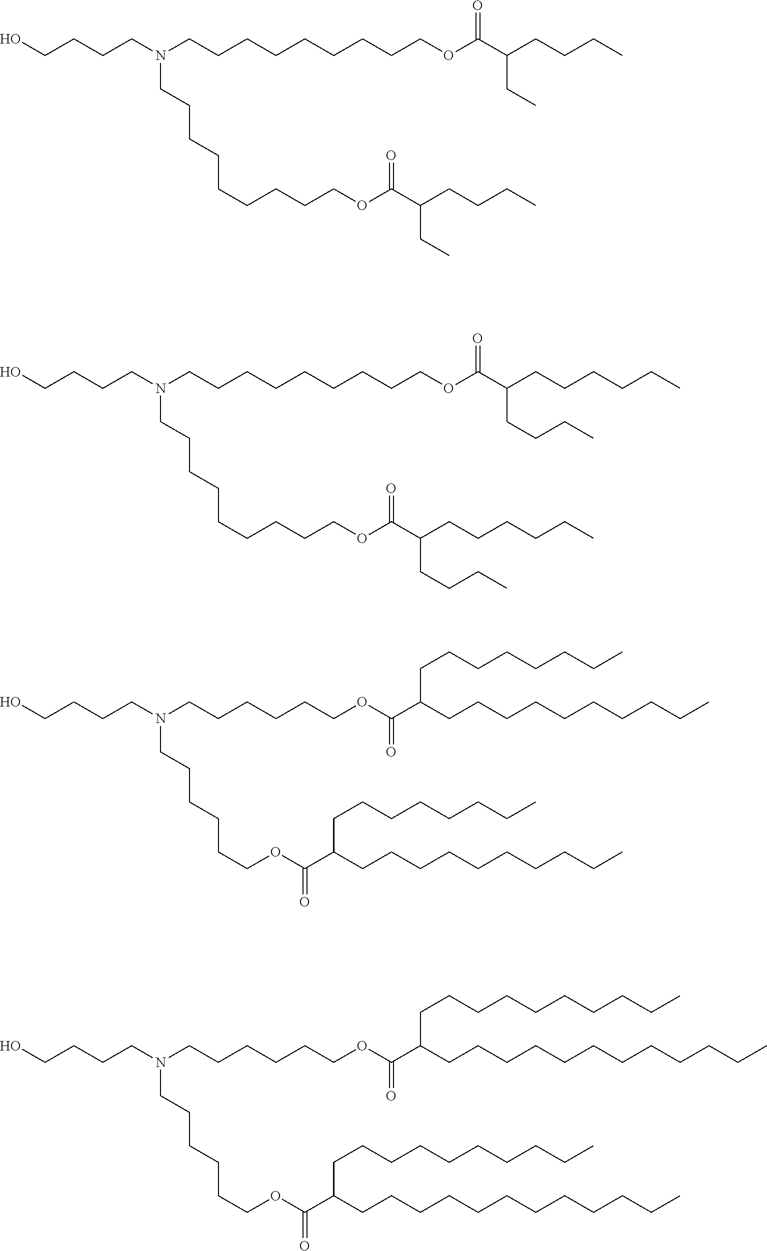 [SMILES: CCCCC(CC)C(=O)OCCCCCCCCCN(CCCCO)CCCCCCCCCOC(=O)C(CC)CCCC ; CCCCCCC(CCCC)C(=O)OCCCCCCCCCN(CCCCO)CCCCCCCCCOC(=O)C(CCCC)CCCCCC ; CCCCCCCCCCC(CCCCCCCC)C(=O)OCCCCCCN(CCCCO)CCCCCCOC(=O)C(CCCCCCCC)CCCCCCCCCC ; CCCCCCCCCCCCC(CCCCCCCCCC)C(=O)OCCCCCCN(CCCCO)CCCCCCOC(=O)C(CCCCCCCCCC)CCCCCCCCCCCC]

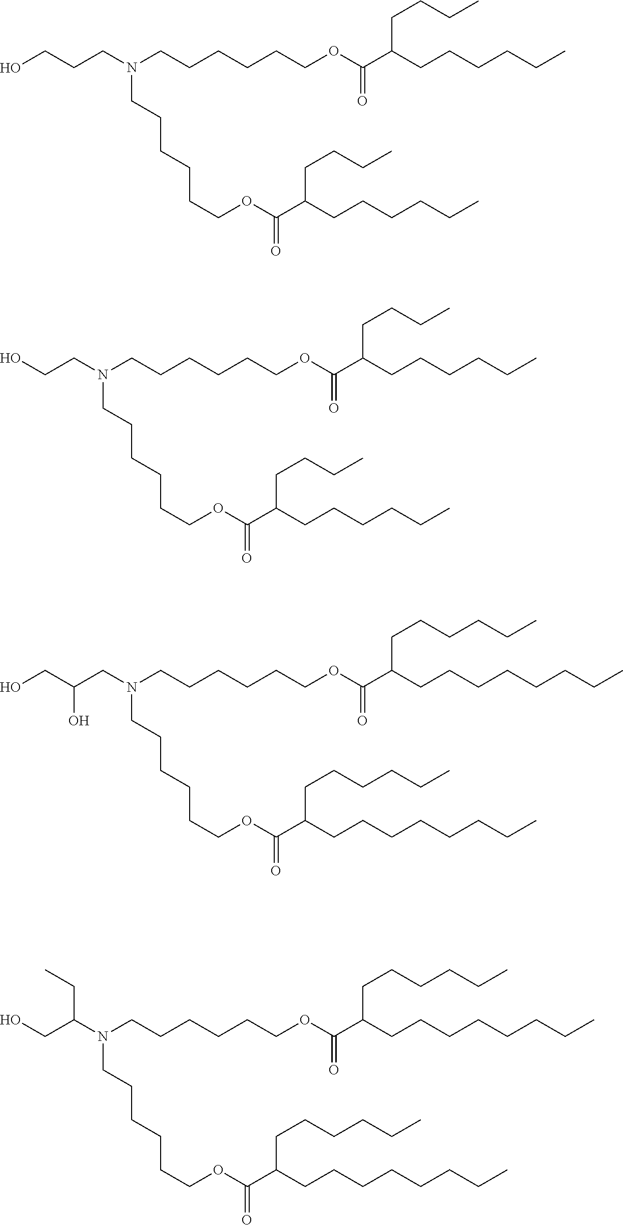 [SMILES: CCCCCCC(CCCC)C(=O)OCCCCCCN(CCCO)CCCCCCOC(=O)C(CCCC)CCCCCC ; CCCCCCC(CCCC)C(=O)OCCCCCCN(CCO)CCCCCCOC(=O)C(CCCC)CCCCCC ; CCCCCCCCC(CCCCCC)C(=O)OCCCCCCN(CCCCCCOC(=O)C(CCCCCC)CCCCCCCC)CC(O)CO ; CCCCCCCCC(CCCCCC)C(=O)OCCCCCCN(CCCCCCOC(=O)C(CCCCCC)CCCCCCCC)C(CC)CO]

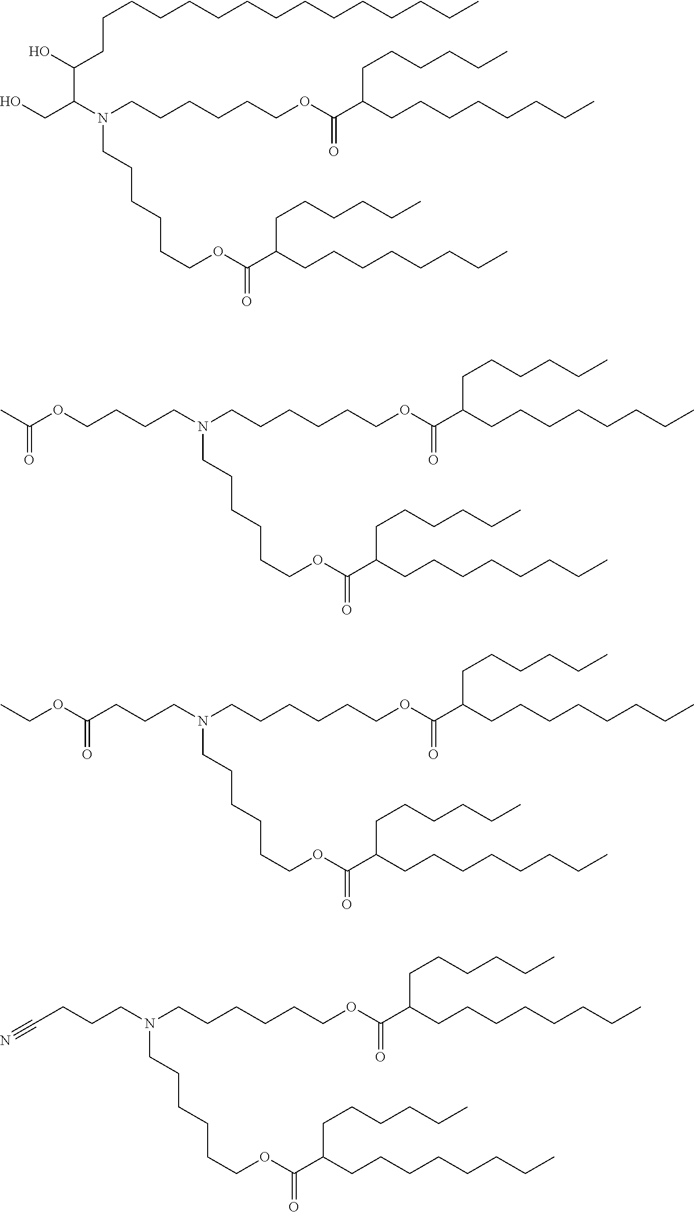

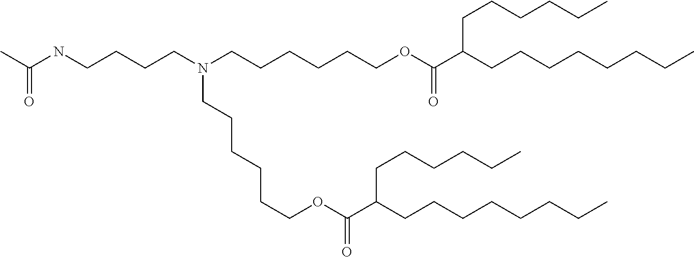 [SMILES: CCCCCCCCC(CCCCCC)C(=O)OCCCCCCN(CCCCCCOC(=O)C(CCCCCC)CCCCCCCC)CCCCNC(C)=O]

In some embodiments, the LNPs comprise a lipid of Formula (III), at least one agent, and one or more excipient selected from neutral lipids, steroids and pegylated lipids. In some embodiments, the lipid of Formula (III) is compound III-3. In some embodiments, the lipid of Formula (III) is compound III-7.

In certain embodiments, the cationic lipid is present in the LNP in an amount from about 30 to about 95 mole percent. In one embodiment, the cationic lipid is present in the LNP in an amount from about 30 to about 70 mole percent. In one embodiment, the cationic lipid is present in the LNP in an amount from about 40 to about 60 mole percent. In one embodiment, the cationic lipid is present in the LNP in an amount of about 50 mole percent. In one embodiment, the LNP comprises only cationic lipids.

In certain embodiments, the LNP comprises one or more additional lipids which stabilize the formation of particles during their formation.

Suitable stabilizing lipids include neutral lipids and anionic lipids.

The term “neutral lipid” refers to any one of a number of lipid species that exist in either an uncharged or neutral zwitterionic form at physiological pH. Representative neutral lipids include diacylphosphatidylcholines, diacylphosphatidylethanolamines, ceramides, sphingomyelins, dihydro sphingomyelins, cephalins, and cerebrosides.

Exemplary neutral lipids include, for example, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoyl-phosphatidyethanol amine (SOPE), and 1,2-dielaidoyl-sn-glycero-3-phophoethanolamine (transDOPE). In one embodiment, the neutral lipid is 1,2-distearoyl-sn-glycero-3-phosphocholine (DSPC).

In some embodiments, the LNPs comprise a neutral lipid selected from DSPC, DPPC, DMPC, DOPC, POPC, DOPE and SM. In various embodiments, the molar ratio of the cationic lipid (e.g., lipid of Formula (I)) to the neutral lipid ranges from about 2:1 to about 8:1.

In various embodiments, the LNPs further comprise a steroid or steroid analogue. A “steroid” is a compound comprising the following carbon skeleton:

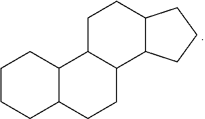 [SMILES: C1CCC2C(C1)CCC1C3CCCC3CCC21]

In certain embodiments, the steroid or steroid analogue is cholesterol. In some of these embodiments, the molar ratio of the cationic lipid (e.g., lipid of Formula (I)) to cholesterol ranges from about 2:1 to 1:1.

The term “anionic lipid” refers to any lipid that is negatively charged at physiological pH. These lipids include phosphatidylglycerol, cardiolipin, diacylphosphatidylserine, diacylphosphatidic acid, N-dodecanoylphosphatidylethanolamines, N-succinylphosphatidylethanolamines, N-glutarylphosphatidylethanolamines, lysylphosphatidylglycerols, palmitoyloleyolphosphatidylglycerol (POPG), and other anionic modifying groups joined to neutral lipids.

In certain embodiments, the LNP comprises glycolipids (e.g., monosialoganglioside GM₁). In certain embodiments, the LNP comprises a sterol, such as cholesterol.

In some embodiments, the LNPs comprise a polymer conjugated lipid. The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-s-DMG) and the like.

In certain embodiments, the LNP comprises an additional, stabilizing-lipid which is a polyethylene glycol-lipid (pegylated lipid). Suitable polyethylene glycol-lipids include PEG-modified phosphatidylethanolamine, PEG-modified phosphatidic acid, PEG-modified ceramides (e.g., PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In one embodiment, the polyethylene glycol-lipid is N-[(methoxy poly(ethylene glycol)₂₀₀₀)carbamyl]-1,2-dimyristyloxlpropyl-3-amine (PEG-c-DMA). In one embodiment, the polyethylene glycol-lipid is PEG-c-DOMG). In other embodiments, the LNPs comprise a pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O-(ω-methoxy(polyethoxy)ethyl)butanedioate (PEG-S-DMG), a pegylated ceramide (PEG-cer), or a PEG dialkoxypropylcarbamate such as ω-methoxy(polyethoxy)ethyl-N-(2,3-di(tetradecanoxy)propyl)carbamate or 2,3-di(tetradecanoxy)propyl-N-(ω-methoxy(polyethoxy)ethyl)carbamate. In various embodiments, the molar ratio of the cationic lipid to the pegylated lipid ranges from about 100:1 to about 25:1.

In some embodiments, the LNPs comprise a pegylated lipid having the following structure (IV):

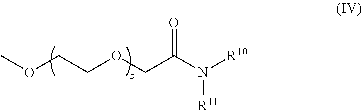

or a pharmaceutically acceptable salt, tautomer or stereoisomer thereof, wherein:
R¹⁰ and R¹¹ are each independently a straight or branched, saturated or unsaturated alkyl chain containing from 10 to 30 carbon atoms, wherein the alkyl chain is optionally interrupted by one or more ester bonds; and
z has mean value ranging from 30 to 60.

In some of the foregoing embodiments of the pegylated lipid (IV), R¹⁰ and R¹¹ are not both n-octadecyl when z is 42. In some other embodiments, R¹⁰ and R¹¹ are each independently a straight or branched, saturated or unsaturated alkyl chain containing from 10 to 18 carbon atoms. In some embodiments, R¹⁰ and R¹¹ are each independently a straight or branched, saturated or unsaturated alkyl chain containing from 12 to 16 carbon atoms. In some embodiments, R¹⁰ and R¹¹ are each independently a straight or branched, saturated or unsaturated alkyl chain containing 12 carbon atoms. In some embodiments, R¹⁰ and R¹¹ are each independently a straight or branched, saturated or unsaturated alkyl chain containing 14 carbon atoms. In other embodiments, R¹⁰ and R¹¹ are each independently a straight or branched, saturated or unsaturated alkyl chain containing 16 carbon atoms. In still more embodiments, R¹⁰ and R¹¹ are each independently a straight or branched, saturated or unsaturated alkyl chain containing 18 carbon atoms. In still other embodiments, R¹⁰ is a straight or branched, saturated or unsaturated alkyl chain containing 12 carbon atoms and R¹¹ is a straight or branched, saturated or unsaturated alkyl chain containing 14 carbon atoms.

In various embodiments, z spans a range that is selected such that the PEG portion of (II) has an average molecular weight of about 400 to about 6000 g/mol. In some embodiments, the average z is about 45.

In other embodiments, the pegylated lipid has one of the following structures:

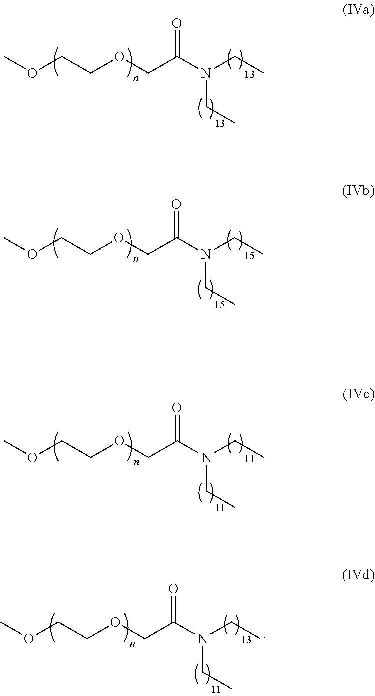

wherein n is an integer selected such that the average molecular weight of the pegylated lipid is about 2500 g/mol.

In certain embodiments, the additional lipid is present in the LNP in an amount from about 1 to about 10 mole percent. In one embodiment, the additional lipid is present in the LNP in an amount from about 1 to about 5 mole percent. In one embodiment, the additional lipid is present in the LNP in about 1 mole percent or about 1.5 mole percent.

In some embodiments, the LNPs comprise a lipid of Formula (I), a nucleoside-modified RNA, a neutral lipid, a steroid and a pegylated lipid. In some embodiments the lipid of Formula (I) is compound I-6. In different embodiments, the neutral lipid is DSPC. In other embodiments, the steroid is cholesterol. In still different embodiments, the pegylated lipid is compound IVa.

In certain embodiments, the LNP comprises one or more targeting moieties that targets the LNP to a stem cell or stem cell population. For example, in one embodiment, the targeting domain is a ligand which directs the LNP to a receptor found on a stem cell surface.

Exemplary LNPs and their manufacture are described in the art, for example in U.S. Patent Application Publication No. US20120276209, Semple et al., 2010, Nat Biotechnol., 28 (2): 172-176; Akinc et al., 2010, Mol Ther., 18 (7): 1357-1364; Basha et al., 2011, Mol Ther, 19 (12): 2186-2200; Leung et al., 2012, J Phys Chem C Nanomater Interfaces, 116 (34): 18440-18450; Lee et al., 2012, Int J Cancer., 131 (5): E781-90; Belliveau et al., 2012, Mol Ther nucleic Acids, 1: e37; Jayaraman et al., 2012, Angew Chem Int Ed Engl., 51 (34): 8529-8533; Mui et al., 2013, Mol Ther Nucleic Acids. 2, e139; Maier et al., 2013, Mol Ther., 21 (8): 1570-1578; and Tam et al., 2013, Nanomedicine, 9 (5): 665-74, each of which are incorporated by reference in their entirety.

The following Reaction Schemes illustrate methods to make lipids of Formula (I), (II) or (III).

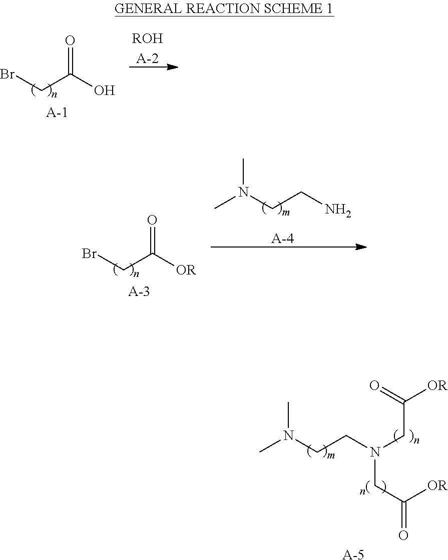

Embodiments of the lipid of Formula (I) (e.g., compound A-5) can be prepared according to General Reaction Scheme 1 (“Method A”), wherein R is a saturated or unsaturated C₁-C₂₄ alkyl or saturated or unsaturated cycloalkyl, m is 0 or 1 and n is an integer from 1 to 24. Referring to General Reaction Scheme 1, compounds of structure A-1 can be purchased from commercial sources or prepared according to methods familiar to one of ordinary skill in the art. A mixture of A-1, A-2 and DMAP is treated with DCC to give the bromide A-3. A mixture of the bromide A-3, a base (e.g., N,N-diisopropylethylamine) and the N,N-dimethyldiamine A-4 is heated at a temperature and time sufficient to produce A-5 after any necessarily workup and or purification step.

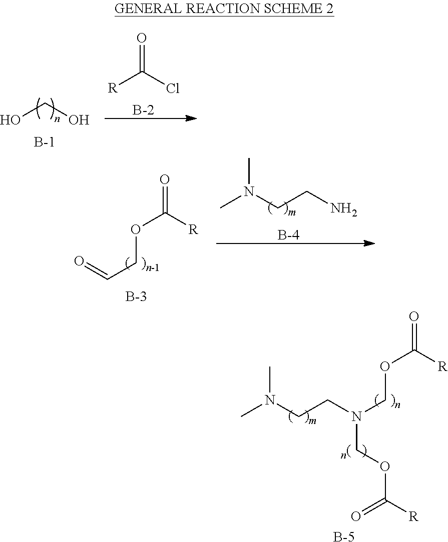

Other embodiments of the compound of Formula (I) (e.g., compound B-5) can be prepared according to General Reaction Scheme 2 (“Method B”), wherein R is a saturated or unsaturated C₁-C₂₄ alkyl or saturated or unsaturated cycloalkyl, m is 0 or 1 and n is an integer from 1 to 24. As shown in General Reaction Scheme 2, compounds of structure B-1 can be purchased from commercial sources or prepared according to methods familiar to one of ordinary skill in the art. A solution of B-1 (1 equivalent) is treated with acid chloride B-2 (1 equivalent) and a base (e.g., triethylamine). The crude product is treated with an oxidizing agent (e.g., pyridinum chlorochromate) and intermediate product B-3 is recovered. A solution of crude B-3, an acid (e.g., acetic acid), and N,N-dimethylaminoamine B-4 is then treated with a reducing agent (e.g., sodium triacetoxyborohydride) to obtain B-5 after any necessary work up and/or purification.

It should be noted that although starting materials A-1 and B-1 are depicted above as including only saturated methylene carbons, starting materials which include carbon-carbon double bonds may also be employed for preparation of compounds which include carbon-carbon double bonds.

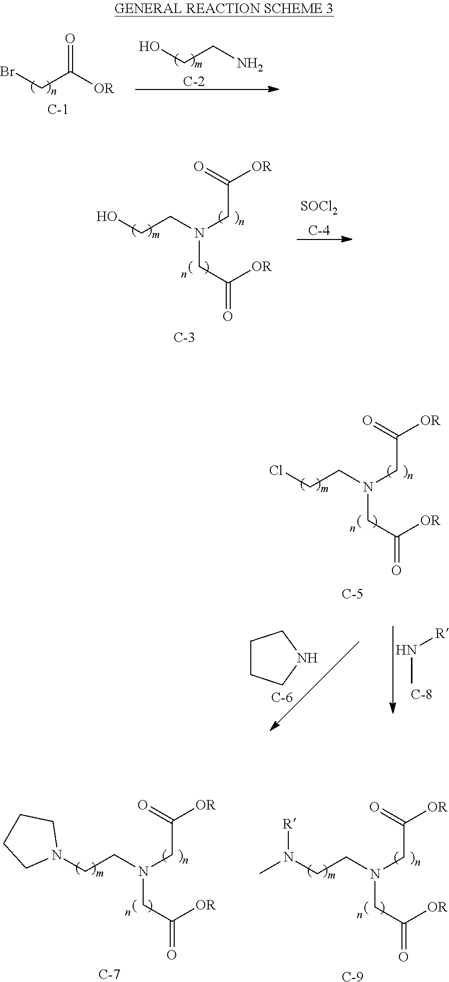

Different embodiments of the lipid of Formula (I) (e.g., compound C-7 or C₉) can be prepared according to General Reaction Scheme 3 (“Method C”), wherein R is a saturated or unsaturated C₁-C₂₄ alkyl or saturated or unsaturated cycloalkyl, m is 0 or 1 and n is an integer from 1 to 24. Referring to General Reaction Scheme 3, compounds of structure C-1 can be purchased from commercial sources or prepared according to methods familiar to one of ordinary skill in the art.

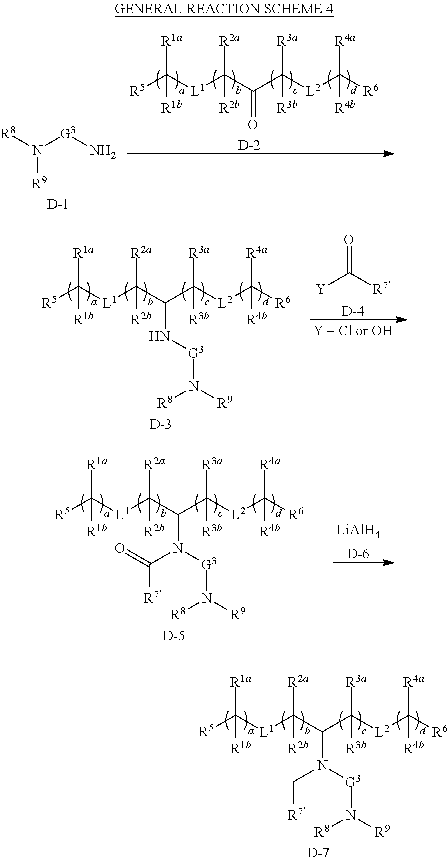

Embodiments of the compound of Formula (II) (e.g., compounds D-5 and D-7) can be prepared according to General Reaction Scheme 4 (“Method D”), wherein R^{1a}, R^{1b}, R^{2a}, R^{2b}, R^{3a}, R^{3b}, R^{4a}, R^{4b}, R⁵, R⁶, R⁸, R⁹, L¹, L², G¹, G², G³, a, b, c and d are as defined herein, and R⁷ represents R⁷ or a C₃-C₁₉ alkyl. Referring to General Reaction Scheme 1, compounds of structure D-1 and D-2 can be purchased from commercial sources or prepared according to methods familiar to one of ordinary skill in the art. A solution of D-1 and D-2 is treated with a reducing agent (e.g., sodium triacetoxyborohydride) to obtain D-3 after any necessary work up. A solution of D-3 and a base (e.g. trimethylamine, DMAP) is treated with acyl chloride D-4 (or carboxylic acid and DCC) to obtain D-5 after any necessary work up and/or purification. D-5 can be reduced with LiAlH4 D-6 to give D-7 after any necessary work up and/or purification.

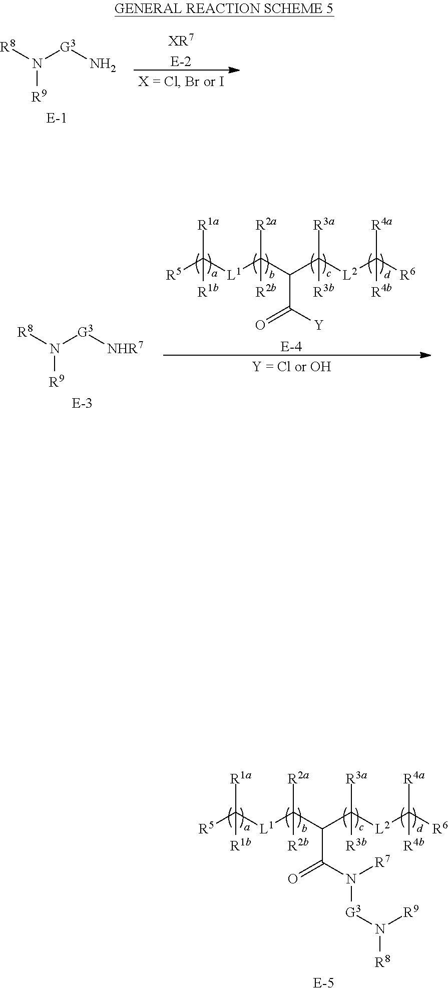

Embodiments of the lipid of Formula (II) (e.g., compound E-5) can be prepared according to General Reaction Scheme 5 (“Method E”), wherein R^{1a}, R^{1b}, R^{2a}, R^{2b}, R^{3a}, R^{3b}, R^{4a}, R^{4b}, R⁵, R⁶, R⁷, R⁸, R⁹, L¹, L², G³, a, b, c and d are as defined herein. Referring to General Reaction Scheme 2, compounds of structure E-1 and E-2 can be purchased from commercial sources or prepared according to methods familiar to one of ordinary skill in the art. A mixture of E-1 (in excess), E-2 and a base (e.g., potassium carbonate) is heated to obtain E-3 after any necessary work up. A solution of E-3 and a base (e.g. trimethylamine, DMAP) is treated with acyl chloride E-4 (or carboxylic acid and DCC) to obtain E-5 after any necessary work up and/or purification.

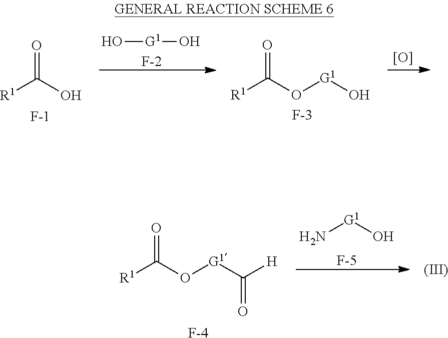

General Reaction Scheme 6 provides an exemplary method (Method F) for preparation of Lipids of Formula (III). G¹, G³, R¹ and R³ in General Reaction Scheme 6 are as defined herein for Formula (III), and G1′ refers to a one-carbon shorter homologue of G1. Compounds of structure F-1 are purchased or prepared according to methods known in the art. Reaction of F-1 with diol F-2 under appropriate condensation conditions (e.g., DCC) yields ester/alcohol F-3, which can then be oxidized (e.g., PCC) to aldehyde F-4. Reaction of F-4 with amine F-5 under reductive amination conditions yields a lipid of Formula (III).

It should be noted that various alternative strategies for preparation of lipids of Formula (III) are available to those of ordinary skill in the art. For example, other lipids of Formula (III) wherein L¹ and L² are other than ester can be prepared according to analogous methods using the appropriate starting material. Further, General Reaction Scheme 6 depicts preparation of a lipids of Formula (III), wherein G¹ and G² are the same; however, this is not a required aspect of the invention and modifications to the above reaction scheme are possible to yield compounds wherein G¹ and G² are different.

It will be appreciated by those skilled in the art that in the process described herein the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (for example, 1-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include-C(O)—R″ (where R″ is alkyl, aryl or arylalkyl), p-methoxybenzyl, trityl and the like. Suitable protecting groups for carboxylic acid include alkyl, aryl or arylalkyl esters. Protecting groups may be added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

### Targeting Domain

In one embodiment, the targeting domain specifically binds to CD117. The targeting domain may comprise a nucleic acid, peptide, antibody, small molecule, organic molecule, inorganic molecule, glycan, sugar, hormone, and the like that targets the particle to a site in particular need of the therapeutic agent. In certain embodiments, the particle comprises multivalent targeting, wherein the particle comprises multiple targeting mechanisms described herein.

In some embodiments, the targeting domain may be co-polymerized with the composition comprising the delivery vehicle. In some embodiments, the targeting domain may be covalently attached to the composition comprising the delivery vehicle, such as through a chemical reaction between the targeting domain and the composition comprising the delivery vehicle. In some embodiments, the targeting domain is an additive in the delivery vehicle. Targeting domains of the instant invention include, but are not limited to, antibodies, antibody fragments, proteins, peptides, and nucleic acids.

In one embodiment, the composition comprises a targeting domain that directs the delivery vehicle to CD117. In some embodiments, the targeting domain is an affinity ligand which specifically binds to CD117.

In one aspect, the present invention relates to composition comprising a delivery vehicle conjugated to a CD117 targeting domain. In certain embodiments, the targeting domain binds to CD117 expressed on the surface of a target stem cell, thereby directing the composition to the target stem cell.

### Peptides

In one embodiment, the targeting domain of the invention comprises a peptide. In certain embodiments, the peptide targeting domain specifically binds to marker of a cell type of interest. In one embodiment, the targeting domain directs the vehicle to an endothelial cell, an immune cell, a stem cell, or another specific cell type of interest. For example, in one embodiment, the targeting domain directs the vehicle to a CD117 expressing stem cell.

The peptide of the present invention may be made using chemical methods. For example, peptides can be synthesized by solid phase techniques (Roberge J Y et al (1995) Science 269:202-204), cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer.

The peptide may alternatively be made by recombinant means or by cleavage from a longer polypeptide. The composition of a peptide may be confirmed by amino acid analysis or sequencing.

The variants of the peptides according to the present invention may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the peptide is an alternative splice variant of the peptide of the present invention, (iv) fragments of the peptides and/or (v) one in which the peptide is fused with another peptide, such as a leader or secretory sequence or a sequence which is employed for purification (for example, His-tag) or for detection (for example, Sv5 epitope tag). The fragments include peptides generated via proteolytic cleavage (including multi-site proteolysis) of an original sequence. Variants may be post-translationally, or chemically modified. Such variants are deemed to be within the scope of those skilled in the art from the teaching herein.

As known in the art the “similarity” between two peptides is determined by comparing the amino acid sequence and its conserved amino acid substitutes of one peptide to a sequence of a second peptide. Variants are defined to include peptide sequences different from the original sequence, preferably different from the original sequence in less than 40% of residues per segment of interest, more preferably different from the original sequence in less than 25% of residues per segment of interest, more preferably different by less than 10% of residues per segment of interest, most preferably different from the original protein sequence in just a few residues per segment of interest and at the same time sufficiently homologous to the original sequence to preserve the functionality of the original sequence. The present invention includes amino acid sequences that are at least 60%, 65%, 70%, 72%, 74%, 76%, 78%, 80%, 90%, or 95% similar or identical to the original amino acid sequence. The degree of identity between two peptides is determined using computer algorithms and methods that are widely known for the persons skilled in the art. The identity between two amino acid sequences is preferably determined by using the BLASTP algorithm [BLAST Manual, Altschul, S., et al., NCBI NLM NIH Bethesda, Md. 20894, Altschul, S., et al., J. Mol. Biol. 215:403-410 (1990)].

The peptides of the invention can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The peptides of the invention may include unnatural amino acids formed by post-translational modification or by introducing unnatural amino acids during translation.

### Nucleic Acids

In one embodiment, the targeting domain of the invention comprises an isolated nucleic acid, including for example a DNA oligonucleotide and a RNA oligonucleotide. In certain embodiments, the nucleic acid targeting domain specifically binds to CD117. For example, in one embodiment, the nucleic acid comprises a nucleotide sequence that specifically binds to CD117.

The nucleotide sequences of a nucleic acid targeting domain can alternatively comprise sequence variations with respect to the original nucleotide sequences, for example, substitutions, insertions and/or deletions of one or more nucleotides, with the condition that the resulting nucleic acid functions as the original and specifically binds to CD117.

In the sense used in this description, a nucleotide sequence is “substantially homologous” to any of the nucleotide sequences describe herein when its nucleotide sequence has a degree of identity with respect to the nucleotide sequence of at least 60%, advantageously of at least 70%, preferably of at least 85%, and more preferably of at least 95%. Other examples of possible modifications include the insertion of one or more nucleotides in the sequence, the addition of one or more nucleotides in any of the ends of the sequence, or the deletion of one or more nucleotides in any end or inside the sequence. The degree of identity between two polynucleotides is determined using computer algorithms and methods that are widely known for the persons skilled in the art. The identity between two amino acid sequences is preferably determined by using the BLASTN algorithm [BLAST Manual, Altschul, S., et al., NCBI NLM NIH Bethesda, Md. 20894, Altschul, S., et al., J. Mol. Biol. 215:403-410 (1990)].

### Antibodies

In one embodiment, the targeting domain of the invention comprises an antibody, or antibody fragment. In certain embodiments, the antibody targeting domain specifically binds to CD117. Such antibodies include polyclonal antibodies, monoclonal antibodies, Fab and single chain Fv (scFv) fragments thereof, bispecific antibodies, heteroconjugates, human and humanized antibodies.

The antibodies may be intact monoclonal or polyclonal antibodies, and immunologically active fragments (e.g., a Fab or (Fab)2 fragment), an antibody heavy chain, an antibody light chain, humanized antibodies, a genetically engineered single chain Fv molecule (Ladner et al, U.S. Pat. No. 4,946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

Such antibodies may be produced in a variety of ways, including hybridoma cultures, recombinant expression in bacteria or mammalian cell cultures, and recombinant expression in transgenic animals. The choice of manufacturing methodology depends on several factors including the antibody structure desired, the importance of carbohydrate moieties on the antibodies, ease of culturing and purification, and cost. Many different antibody structures may be generated using standard expression technology, including full-length antibodies, antibody fragments, such as Fab and Fv fragments, as well as chimeric antibodies comprising components from different species. Antibody fragments of small size, such as Fab and Fv fragments, having no effector functions and limited pharmokinetic activity may be generated in a bacterial expression system. Single chain Fv fragments show low immunogenicity.

### Conjugation

In various embodiments of the invention, the delivery vehicle (e.g., LNP) is conjugated to the CD117 targeting domain. Exemplary methods of conjugation can include, but are not limited to, covalent bonds, electrostatic interactions, and hydrophobic (“van der Waals”) interactions. In one embodiment, the conjugation is a reversible conjugation, such that the delivery vehicle can be disassociated from the targeting domain upon exposure to certain conditions or chemical agents. In another embodiment, the conjugation is an irreversible conjugation, such that under normal conditions the delivery vehicle does not dissociate the targeting domain.

In some embodiments, the conjugation comprises a covalent bond between an activated polymer conjugated lipid and the targeting domain. The term “activated polymer conjugated lipid” refers to a molecule comprising a lipid portion and a polymer portion that has been activated via functionalization of a polymer conjugated lipid with a first coupling group. In one embodiment, the activated polymer conjugated lipid comprises a first coupling group capable of reacting with a second coupling group. In one embodiment, the activated polymer conjugated lipid is an activated pegylated lipid. In one embodiment, the first coupling group is bound to the lipid portion of the pegylated lipid. In another embodiment, the first coupling group is bound to the polyethylene glycol portion of the pegylated lipid. In one embodiment, the second functional group is covalently attached to the targeting domain.

The first coupling group and second coupling group can be any functional groups known to those of skill in the art to together form a covalent bond, for example under mild reaction conditions or physiological conditions. In some embodiments, the first coupling group or second coupling group are selected from the group consisting of maleimides, N-hydroxysuccinimide (NHS) esters, carbodiimides, hydrazide, pentafluorophenyl (PFP) esters, phosphines, hydroxymethyl phosphines, psoralen, imidoesters, pyridyl disulfide, isocyanates, vinyl sulfones, alpha-haloacetyls, aryl azides, acyl azides, alkyl azides, diazirines, benzophenone, epoxides, carbonates, anhydrides, sulfonyl chlorides, cyclooctyne, aldehydes, and sulfhydryl groups. In some embodiments, the first coupling group or second coupling group is selected from the group consisting of free amines (—NH₂), free sulfhydryl groups (—SH), free hydroxide groups (—OH), carboxylates, hydrazides, and alkoxyamines. In some embodiments, the first coupling group is a functional group that is reactive toward sulfhydryl groups, such as maleimide, pyridyl disulfide, or a haloacetyl. In one embodiment, the first coupling group is a maleimide.

In one embodiment, the second coupling group is a sulfhydryl group. The sulfhydryl group can be installed on the targeting domain using any method known to those of skill in the art. In one embodiment, the sulfhydryl group is present on a free cysteine residue. In one embodiment, the sulfhydryl group is revealed via reduction of a disulfide on the targeting domain, such as through reaction with 2-mercaptoethylamine. In one embodiment, the sulfhydryl group is installed via a chemical reaction, such as the reaction between a free amine and 2-iminothilane or N-succinimidyl S-acetylthioacetate (SATA).

In some embodiments, the polymer conjugated lipid and the targeting domain are functionalized with groups used in “click” chemistry. Bioorthogonal “click” chemistry comprises the reaction between a functional group with a 1,3-dipole, such as an azide, a nitrile oxide, a nitrone, an isocyanide, and the link, with an alkene or an alkyne dipolarophiles. Exemplary dipolarophiles include any strained cycloalkenes and cycloalkynes known to those of skill in the art, including, but not limited to, cyclooctynes, dibenzocyclooctynes, monofluorinated cyclcooctynes, difluorinated cyclooctynes, and biarylazacyclooctynone

### Combinations

In one embodiment, the composition of the present invention comprises a combination of agents described herein (e.g., a combination of a CD117-LNP comprising a p53 upregulated modulator of apoptosis (PUMA) mRNA and diphenhydramine or an antihistamine agent). In certain embodiments, a composition comprising a combination of agents described herein has an additive effect, wherein the overall effect of the combination is approximately equal to the sum of the effects of each individual agent. In other embodiments, a composition comprising a combination of agents described herein has a synergistic effect, wherein the overall effect of the combination is greater than the sum of the effects of each individual agent.

A composition comprising a combination of agents comprises individual agents in any suitable ratio. For example, in one embodiment, the composition comprises a 1:1 ratio of two individual agents. However, the combination is not limited to any particular ratio. Rather any ratio that is shown to be effective is encompassed.

### Therapeutic Methods

In some embodiments, the invention provides methods for preconditioning of HSCs. In some embodiments, the invention provides methods for treatment of a disease or disorder in a subject.

In some embodiments, the invention provides methods for delivery of an apoptosis inducing mRNA or protein to patient HSCs to deplete patient HSC for the treatment of a disease or disorder. In some embodiments, the apoptosis inducing mRNA encodes PUMA, Bak-1, Cas-3 or Cas-9, or a functional fragment or variant thereof. In some embodiments, the apoptosis inducing mRNA comprises SEQ ID NO:5, SEQ ID NO: 6, SEQ ID NO:7 or SEQ ID NO:8, or a functional fragment or variant thereof.

In some embodiments, the invention provides methods for delivery of an apoptosis inducing mRNA or protein to patient HSCs to precondition the patient for HSCT therapy. In some embodiments, delivery of the apoptosis inducing mRNA or protein to the HSCs of the patient prior to HSCT promotes engraftment of therapeutic HSCs.

In some embodiments, the invention provides methods for preconditioning a patient for HSCT therapy comprising providing a combination of an apoptosis inducing mRNA or protein, and an antihistamine agent, an anti-inflammatory agent, or a combination of an antihistamine agent and an anti-inflammatory agent to the patient. In some embodiments the apoptosis inducing mRNA or protein, and the antihistamine agent and/or anti-inflammatory agent are provided to a subject substantially concurrently. In some embodiments, the apoptosis inducing mRNA or protein and the antihistamine agent and/or anti-inflammatory agent are provided to a subject individually, but within a time period of 2 weeks or less from the administration of the first agent to the administration of the final agent. In some embodiments, the apoptosis inducing mRNA or protein and the antihistamine agent and/or anti-inflammatory agent are provided to a subject individually, but within a time period of 2 days or less from the administration of the first agent to the administration of the final agent. In some embodiments, the apoptosis inducing mRNA or protein and the antihistamine agent and/or anti-inflammatory agent are provided to a subject individually, but within a time period of 2 hours or less from the administration of the first agent to the administration of the final agent. For example, in some embodiments, the apoptosis inducing mRNA or protein is administered within a time period of 2 hours or less from the administration of the antihistamine agent or anti-inflammatory agent. In some embodiments, the apoptosis inducing mRNA or protein, the antihistamine agent and the anti-inflammatory agent are each administered within a time period of 2 hours. In some embodiments, the antihistamine agent is administered between 1 week and 1 minute prior to administration of the apoptosis inducing mRNA or protein, and the anti-inflammatory agent is administered between 1 minute and 1 week following administration of the apoptosis inducing mRNA or protein. In some embodiments, the antihistamine agent is administered between 1 day and 1 minute prior to administration of the apoptosis inducing mRNA or protein, and the anti-inflammatory agent is administered between 1 minute and 1 day following administration of the apoptosis inducing mRNA or protein. In some embodiments, the antihistamine agent is administered between 1 minutes and 1 hour prior to administration of the apoptosis inducing mRNA, and the anti-inflammatory agent is administered between 1 minute and 1 hour following administration of the apoptosis inducing mRNA or protein. In one embodiment, an antihistamine agent is administered about 15 min before, and an anti-inflammatory agent is administered about 5 min after, administration of an apoptosis inducing mRNA.

Exemplary antihistamine agents that can be administered in combination with the apoptosis inducing mRNA or protein according to the method of the invention include, but are not limited to, Acrivastine, Alimemazine, Amitriptyline, Amoxapine, Aripiprazole, Azelastine, Bilastine, Bromodiphenhydramine, Brompheniramine, Buclizine, Carbinoxamine, Cetirizine, Chlophedianol, Chlorodiphenhydramine, Chlorpheniramine, Chlorpromazine, Chlorprothixene, Chloropyramine, Cinnarizine, Clemastine, Clomipramine, Clozapine, Cyclizine, Cyproheptadine, Desloratadine, Dexbrompheniramine, Dexchlorpheniramine, Dimenhydrinate, Dimetindene, Diphenhydramine, Dosulepin, Doxepin, Doxylamine, Ebastine, Embramine, Fexofenadine, Fluoxetine, Hydroxyzine, Imipramine, Ketotifen, Levocabastine, Levocetirizine, Levomepromazine, Loratadine, Maprotiline, Meclizine, Mianserin, Mirtazapine, Olanzapine, Olopatadine, Orphenadrine, Periciazine, Phenindamine, Pheniramine, Phenyltoloxamine, Promethazine, Pyrilamine, Quetiapine, Rupatadine, Setastine, Setiptiline, Trazodone, Tripelennamine, and Triprolidine.

Exemplary anti-inflammatory agents that can be administered in combination with the apoptosis inducing mRNA or protein according to the method of the invention include, but are not limited to, corticosteroids such as dexamethasone, methylprednisolone, cortisone, hydrocortisone and prednisone and non-steroidal anti-inflammatory drugs (NSAIDs) such as ibuprofen, naproxen, celecoxib, diclofenac, indomethacin, oxaprozin, and piroxicam.

In some embodiments, delivery of the apoptosis inducing mRNA or protein to the HSCs of the patient prior to HSCT promotes engraftment of therapeutic HSCs.

Exemplary diseases or disorders that can be treated using the methods of the invention include, but are not limited to, blood monogenic disorders, genetic defects (e.g., beta thalassemia or sickle cell disease), bone marrow genetic defects, cancers, platelet disorders, red cell disorders, immunodeficiencies, non-hematologic diseases (e.g., cystic fibrosis), metabolic disease and autoimmune diseases.

Exemplary bone marrow genetic defects include, but are not limited to, leukemia, aplastic anemia, myeloproliferative disorders, an inherited bone marrow failure syndrome (IBMFS) such as Fanconi anemia, dyskeratosis congenital, Shwachman-Diamond syndrome, Diamond-Blackfan anemia, severe congenital neutropenia, a primary immunodeficiency such as X1-SCID and Wiskott-Aldrich syndrome, an erythroid disorder such as sickle cell disease (SCD), pyruvate kinase deficiency, or a lysosomal storage diseases such as Fabry disease and Pompe disease.

Exemplary inflammatory conditions and autoimmune diseases include, but are not limited to, rheumatoid arthritis, systemic lupus erythematosus, alopecia areata, anklosing spondylitis, antiphospholipid syndrome, autoimmune Addison's disease, autoimmune hemolytic anemia, autoimmune hepatitis, autoimmune inner ear disease, autoimmune lymphoproliferative syndrome (alps), autoimmune thrombocytopeni purpura (ATP), Behcet's disease, bullous pemphigoid, cardiomyopathy, celiac sprue-dermatitis, chronic fatigue syndrome immune deficiency, syndrome (CFIDS), chronic inflammatory demyelinating polyneuropathy, cicatricial pemphigoid, cold agglutinin disease, Crest syndrome, Crohn's disease, Dego's disease, dermatomyositis, dermatomyositis-juvenile, discoid lupus, essential mixed cryoglobulinemia, fibromyalgia-fibromyositis, Grave's disease, Guillain-Barre, Hashimoto's thyroiditis, idiopathic pulmonary fibrosis, idiopathic thrombocytopenia purpura (ITP), Iga nephropathy, insulin dependent diabetes (Type I), juvenile arthritis, Meniere's disease, mixed connective tissue disease, multiple sclerosis, myasthenia gravis, pemphigus vulgaris, pernicious anemia, polyarteritis nodosa, polychondritis, polyglancular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, sarcoidosis, scleroderma, Sjogren's syndrome, stiff-man syndrome, Takayasu arteritis, temporal arteritis/giant cell arteritis, ulcerative colitis, uveitis, vasculitis, vitiligo, Wegener's granulomatosi, and other organ-specific inflammatory disorders.

To practice the methods of the invention; the skilled artisan would understand, based on the disclosure provided herein, how to formulate and administer the appropriate composition to a subject. The present invention is not limited to any particular method of administration or treatment regimen.

The invention encompasses delivery of a CD117 targeted LNP delivery vehicle, comprising at least one PUMA mRNA further comprising a miR-122 binding site, wherein the delivery vehicle is conjugated to a CD117 targeting domain.

It will be appreciated by one of skill in the art, when armed with the present disclosure including the methods detailed herein, that the invention is not limited to treatment of diseases or disorders that are already established. Particularly, the disease or disorder need not have manifested to the point of detriment to the subject; indeed, the disease or disorder need not be detected in a subject before treatment is administered. That is, significant signs or symptoms of diseases or disorders do not have to occur before the present invention may provide benefit. Therefore, the present invention includes a method for preventing diseases or disorders, in that a composition, as discussed previously elsewhere herein, can be administered to a subject prior to the onset of diseases or disorders, thereby preventing diseases or disorders.

One of skill in the art, when armed with the disclosure herein, would appreciate that the prevention of a disease or disorder, encompasses administering to a subject a composition as a preventative measure against the development of, or progression of, a disease or disorder.

One of skill in the art will appreciate that the compositions of the invention can be administered singly or in any combination. Further, the compositions of the invention can be administered singly or in any combination in a temporal sense, in that they may be administered concurrently, or before, and/or after each other. One of ordinary skill in the art will appreciate, based on the disclosure provided herein, that the compositions of the invention can be used to prevent or to treat a disease or disorder, and that a composition can be used alone or in any combination with another composition to affect a therapeutic result. In various embodiments, any of the compositions of the invention described herein can be administered alone or in combination with other modulators of other molecules associated with diseases or disorders.

In one embodiment, the invention includes a method comprising administering a combination of compositions described herein. In certain embodiments, the method has an additive effect, wherein the overall effect of the administering a combination of compositions is approximately equal to the sum of the effects of administering each individual inhibitor. In other embodiments, the method has a synergistic effect, wherein the overall effect of administering a combination of compositions is greater than the sum of the effects of administering each individual composition.

The method comprises administering a combination of composition in any suitable ratio. For example, in one embodiment, the method comprises administering two individual compositions at a 1:1 ratio. However, the method is not limited to any particular ratio. Rather any ratio that is shown to be effective is encompassed.

### Pharmaceutical Compositions

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, intravenous, intracerebroventricular, intradermal, intramuscular, or another route of administration. Other contemplated formulations include projected nanoparticles, liposomal preparations, resealed erythrocytes containing the active ingredient, and immunogenic-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

In addition to the active ingredient, a pharmaceutical composition of the invention may further comprise one or more additional adjuvants. Exemplary adjuvants include, but are not limited to, aluminum-based adjuvant and monophosphoryl lipid A.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intradermal, intrasternal injection, intratumoral, intravenous, intracerebroventricular and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations that are useful include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, PA), which is incorporated herein by reference.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: In Vivo Hematopoietic Stem Cell Modification by mRNA Delivery

Hematopoietic stem cells (HSC) are the source of all blood cells over the course of an individual's lifetime. Effecting changes in bona fide long-term hematopoietic stem cells (LT-HSC) offers the ability to permanently change blood cells, despite the constant turnover of lineage committed progeny. A novel lipid nanoparticle (LNP) targeted to stem cell factor receptor (CD117) was developed (CD117/LNP-mRNA), which are capable of delivering nucleoside-modified messenger RNA (mRNA) to LT-HSC. The efficacy of this LT-HSC targeted LNP was evaluated in cell specific genomic editing and mRNA delivery. Efficient genome editing utilizing Cre recombinase was observed both ex vivo and in vivo, respectively, in ~99% and ~64% of Lin−Scal+cKit+bone marrow (BM) cells. Targeting of multi-potent and self-renewing progenitors was shown through primary and secondary BM transplantation. Furthermore, in vivo delivery of pro-apoptotic mRNA by i.v. administration of a single dose of CD117/LNP pro-apoptotic mRNA at 0.05 mg/kg (1 μg per animal) in mice effected HSC function and permitted non-genotoxic conditioning for HSC transplant (HSCT).

These results indicate that LNP loaded with diverse mRNA cargos can access HSC in the BM niche in vivo, with a single-dose systemic injection. Delivery efficacy to bona fide long-term HSC in the BM niche is greatly increased by conjugation with targeting agents, like anti-CD117 antibody. Here the data showed that LNP loaded with a Cre mRNA cargo can induce durable genome edits in self renewing and multipotent long-term HSC ex vivo and in vivo and at, or above, the level required for cure of most non-malignant hematopoietic disorders. Additionally, it is demonstrated for the first time that a genetic medicine, targeted LNP-mRNA, can leverage the understanding of HSC biology (Mcl-1 pathway dependence) to effect cellular state change in vivo with physiologic effects. This system was utilized to deplete HSC in vivo without additional genotoxic conditioning regimens such as alkylating chemotherapy or radiation, thus bypassing the acute and chronic toxicity seen clinically, which include pulmonary disease, liver disease, and sterility (Campbell et al., 2010, Blood, 116:1433-1442; Leiper et al., 2002, Br J Haematol, 118:23-43; Cohen et al., 2008, Bone Marrow Transplant, 41 Suppl 2: S43-48).

These findings will revolutionize medicine in two ways. First, the cure of monogenic disorders, such as hemoglobinopathies and other red cell disorders, platelet disorders, immunodeficiencies, and non-hematologic diseases such as cystic fibrosis and metabolic disease with a simple i.v. infusion. Novel delivery systems will yield the promise of decades of concerted genetic and biomedical research. Second, effecting cell-type specific state changes in vivo with minimal risk will allow previously impossible manipulations of physiology.

The ability to target HSC in vivo offers a non-genotoxic conditioning regiment for hematopoietic stem cell transplantation (HSCT) and could be the basis for future in vivo genome editing of genetic disorders, abrogating the need for HSCT. Such delivery systems may help translate the promise of decades of concerted genetic and biomedical research to treat a wide array of human diseases.

The materials and methods used for the experiments are now described.

### RNA Synthesis and Preparation of Targeted LNP-mRNA

Gene sequences for Firefly Luciferase (luc2), Cre Recombinase (cre), and enhanced Green Fluorescent Protein (eGFP) were sourced from SnapGene software plasmids pGL4.10 [luc2], pCMV6-Entry-Cre, and pEGFP, respectively, then codon-optimized. The PUMA gene was codon-optimized from the NCBI reference sequence NM_133234.3. Sequences for murine BAK1, CASP3, and CASP7 were codon-optimized from NCBI reference sequences NM_007523.3, NM_009810.3, and NM 007611 respectively. Constructs incorporating three miR-122 binding sites in the 3′UTR were directly reproduced from Jain et al (Jain et al., 2018, Nucleic Acid Ther. 28, 285-296). The ABE sequence (ABE8e-NRCH) and HBBS-targeting sgRNA sequences were replicated from Newby et al (Newby et al., Nature 595, 295-302) and the sgRNA was purchased from Synthego (Redwood City, CA, USA). All sequences are in Table 1.

Each coding sequence was cloned into an IVT template plasmid carrying a T7 promoter, 5′ and 3′ UTR elements, Kozak consensus sequence, and 101 poly(A) tail. DNA synthesis, cloning and industrial grade endotoxin-free plasmid preparation service was provided by GenScript (Piscataway, NJ, USA). IVT-mRNA was produced using linearized IVT template plasmid and the MEGAScript T7 kit (Invitrogen AMB13345) and formulated with nucleoside-modified m1 P-5′-triphosphate (TriLink N-1081, San diego, CA) instead of UTP. 5′ Capping of the IVT-mRNAs were performed co-transcriptionally using the trinucleotide cap1 analog, CleanCap® Reagent AG (3′ OMe) (TriLink N-7413, San Diego, CA, USA). Single-stranded IVT-mRNA was the purified by cellulose purification, as previously described (Baiersdorfer, et al., 2019, Mol Ther Nucleic Acids, 15:26-35). All mRNAs were analyzed by agarose gel electrophoresis and were stored at −20° C. Cellulose purified m 1W-containing RNAs were encapsulated in LNP using a self-assembly process as previously described (Maier, et al., 2013, Mol Ther, 21 (8): 1570-8), briefly an ethanolic lipid mixture of ionizable cationic lipid, phosphatidylcholine, cholesterol, and polyethylene glycol-lipid was rapidly mixed with an aqueous solution containing the mRNA at acidic pH. The RNA-loaded particles were characterized by dynamic light scattering using a Zetasizer Nano ZS (Malvern Instruments, Malvern, UK) and a Ribogreen assay. The mean hydrodynamic diameter of these LNP-mRNAs was approximately 80 nm with a polydispersity index of 0.02-0.06 and an encapsulation efficiency of ~95%.

To prepare antibody-targeted LNP-mRNA, LNP-mRNA were conjugated with purified rat anti-mouse CD117 (c-kit), clone 2B8 (BioLegend 93235, San Diego, CA, USA) or mouse anti-human CD117 (c-kit), clone 104D2 (BioLegend 95747), and control isotype-matched IgG via SATA-maleimide chemistry, as described previously (Parhiz, et al., 2018, J Control Release, 291:106-115). Briefly, LNP was modified with maleimide functioning groups (DSPE-PEG-mal) by a post-insertion technique. The antibody was functionalized with SATA (N-succinimidyl S-acetylthioacetate) (Millipore Sigma, 26102) to introduce sulfhydryl groups allowing conjugation to maleimide. SATA was deprotected using 0.5 M hydroxylamine followed by removal of the unreacted components by G-25 Sephadex Quick Spin Protein columns (Roche Applied Science, Indianapolis, IN, USA). The reactive sulfhydryl group on the antibody was then conjugated to maleimide moieties using thioether conjugation chemistry. Purification was carried out using Sepharose CL-4B gel filtration columns (Millipore Sigma). mRNA content was calculated by performing a modified Quant-iT RiboGreen RNA assay (Invitrogen). After addition of the targeting ligand, all the targeted and non-targeted LNP preparations were kept at 4° C. and were used within three days of preparation.

### In Vitro Cell Transfection Studies and Luciferase Assay

LNPs carrying reporter luciferase IVT-mRNA were added at increasing concentrations to the cells and incubated for 24 h. Plates were then washed with PBS, lysed in luciferase cell culture lysis reagent (Promega, Madison, WI, USA), and the luciferase activity as luminescence (Luciferase assay system, Promega) was measured.

### Bioluminescence Imaging

C57BL/6J mice were i.v. injected with control IgG/LNP-Luc, CD117/LNP-Luc or CD117/LNP-Luc-miRts formulations. At 5 hours post-injection, bioluminescence imaging was carried out as described previously (Parhiz, et al., 2018, J Control Release, 291:106-115) using an IVIS Spectrum imaging system (Caliper Life Sciences, Waltham, MA, USA). D-luciferin sodium salt (Regis Technologies, Morton Grove, IL, USA) dissolved in PBS was administered to mice intraperitoneally at a dose of 150 mg/kg. After 5 min, the mice were euthanized; desired tissues were harvested, washed with PBS, and immediately placed on the imaging platform. Harvested femurs were slightly crushed by spatula to expose the bone marrow for imaging. Tissue luminescence was measured on the IVIS imaging system using an exposure time of 5 seconds or longer to ensure that the signal obtained was within operative detection range.

### In Vitro Cell Treatment

Bone marrow cells were isolated from femurs of animals, after removal of muscle and connective tissues, by mechanical crushing, which maximizes cell recovery. BM cells were resuspended in 4% FBS PBS and RBC were lysed using ACK lysis buffer at room temperature, according to manufacturer protocol, filtered through a 40 μM sterile strainer (Corning) and washed with 4% FBS PBS solution.

After lysis, cells were counted and assessed for viability by AOPI staining, using a Nexcelom Cellometer cell viability counter (Perkin Elmer) and seeded at a 1.5E+06/mL concentration in Stemspan (Stem Cell Technologies) supplemented with 50 ng/ml mSCF, 6 ng/ml mIL3, and 10 ng/ml mIL6 (all from Peprotech), LNP formulations were added at the time of seeding and for up to 18 hours, depending upon assay.

### Human Erythroid Progenitor Cells (ErPC) Editing and Differentiation

Human CD34+ cells were isolated from blood products by immunomagnetic-separation using the CD34 MicroBead Kit (130-046-702) from Miltenyi Biotec Inc. (Auburn, CA). ErPC were obtained through expansion of CD34+ cells, using a culture system previously described (Leiper, 2002, Br. J. Haematol. 118, 23-43) and frozen after 6-10 days. Upon thawing cells were let recover for 48 hrs and exposed to anti-human CD117/LNP-ABE and CD117/LNP-sgRNA formulation (3 to 10 pg/cells dose, at 1:1 weight ratio) at a 1.5E+06/mL cell concentration for 6 hours. Cell viability was measured before and after treatment by AOPI staining. ErPC were let expand 24 prior to inducing differentiation. Differentiated erythroblasts were collected after 7 days.

### Sickling Assay

The degree of cell sickling was measured using a modified version of a method already described (28). Briefly, 1E+06 differentiated erythroblasts were suspended in 100 L of isotonic TES, supplemented with 10 mM glucose and 0.2% bovine serum albumin, in individual wells of a Costar polystyrene 96-well microplate (No 9017; Corning, Corning, NY). The microplate was then transferred to a Thermomixer R shaker-incubator (Eppendorf, Enfield, CT), and maintained under hypoxia (2.5% Oxygen gas, balance Nitrogen gas), with continuous agitation at 900 rpm, at 37 C for 2 hours. At conclusion, aliquots (~20 μL) of each sample were collected in 2% glutaraldehyde solution for immediate fixation without exposure to air. Subsequently, fixed cell suspensions were introduced into specialized glass microslides (Dawn Scientific, Inc., Newark, NJ) (29) for acquisition of bright field images (at 20× magnification) of single layer cells on a Zeiss Axiovert 200M inverted microscope (Carl Zeiss Microscopy, LLC, White Plains, NY), fitted with an Infinity 2 camera (Teledyne Luminera, Ottawa, ON, Canada) and the coupled Image Capture software.

### Quantification of HbbG-Makassar Protein

Single chain quantification of individual globins was assessed by reverse-phase HPLC on clarified cell lysates obtained by disrupting cell pellets in water. Hemoglobin samples were injected in a Nexera apparatus from Shimadzu Scientific Instruments, Inc. (Columbia, MD) using a 250 mm×4.6 mm Aeris 3.6 μm C4 200 Å column from Phenomenex (Torrance, CA) and a gradient from 32% to 47% of 0.1% trifluoroacetic acid in acetonitrile in water over 60 minutes, with UV detection at 215 nm. Serial dilutions of a solution with known concentrations of Hbb A-F—S—C (Helena Laboratories, Beaumont, TX) were used to generate a calibration curve, where the peak areas were plotted against the concentration values. Types and relative quantity of Hbs in samples were assessed by comparison to standard hemoglobin controls.

### Quantification of Base Editing

Genomic DNA was extracted with QIAamp DNA Mini Kit (56304) from Qiagen (Hilden, Germany) or QuickExtract™ DNA Extraction Solution (QE09050 and QE0905T), from Lucigen (Middelton, WI). Quantification of base editing was performed on 50 ng of genomic DNA upon amplification of the region that includes the binding site of the gRNA using KAPA2G Fast ReadyMix from Kapa Biosystem (Wilmington, MA, USA). After Sanger sequencing (Azenta), the editing percentage was calculated using EditR (for A>G conversion, Adenine Base editor targeting), following a workflow previously described (30).

### CFU Assay

Bone marrow harvested from animals was seeded using a at 30,000 cells/well in complete Methocult media (#M3434, Stem Cell Technologies), on meniscus-free 6-well SmartDish plates (Stem Cell Technologies), using 16 gouge blunt end needles (Stem Cell Technologies). Colonies were incubated for 2 weeks at 37° C. in CO2 incubators. Colonies were imaged using Evos FL Auto (Life Technologies) microscope and analyzed using superimposed images of bright field and Texas Red filter pictures.

Animal Treatments for Ex Vivo/Competitive Transplants, PUMA Preconditioned Transplants, i.v. Injections, and Perfusion

C57BL/6 CD45.1 recipients were lethally irradiated with 2 consecutive cycles (5 Gy each, 4 hours apart) of high intensity X-ray irradiation source. Each recipient received 2-3 million donor RBC lysed bone marrow cells after ex-vivo treatment with LNP formulations (18 hr). CD117/LNP-PUMA pre-conditioning was provided by i.v. injection 6.5 days prior to bone marrow transplant of 10E+06 RBC-depleted GFP+C57BL/6 CD45.2 BM cells. In vivo injections of LNP formulations and BM infusions were provided by retroorbital vein on mice under isoflurane-induced anesthesia, using a precision vaporizer.

### Lung and Liver Perfusion

In preparation for organ perfusion mice were induced to general anesthesia by injected by IP injection of a 200 mg/kg ketamine and 20 mg/kg xylazine solution in PBS. Upon reaching complete loss of footpad reflexes mouse abdominal cavity was cut open and the ribcage was dissected to open the chest cavity. The heart was slowly infused with 10 mL of 1% FBS PBS solution using a 27 Gx ½″-0.3χ13 mm Hypodermic Needle (Beckman Dickinson) upon interruption of portal vein flow. Lungs and liver were rapidly removed and first incubated in 1× Buffer S (provided with GentleMACS Lung Dissociation Kit, Miltenyi) solution or DMEM (Cellgro) media, respectively. After initial homogenization, lung and liver were process following manufacturing instructions for GentleMACS Lung or Liver Dissociation Kits, Miltenyi. Tissues were homogenized using GentleMACs Dissociator system (Miltenyi), following the recommended programs. Cell pellets underwent 1-2 cycles of RBC lysis with ACK buffer, followed by ice cold PEB (PBS phosphate-buffered saline (PBS), pH 7.2, 0.5% bovine serum albumin (BSA), and 2 mM EDTA) buffer washes. Cell number and viability was assessed by AOPI staining, using a Nexcelom Cellometer cell viability counter (Perkin Elmer).

### Flow Cytometry for Analyses for BM and Peripheral Blood

Monthly assessments of peripheral blood cell Td tomato marking were carried out by direct measurement of Td tomato expression in whole blood for the RBC compartment, or after RBC lysis, using ACK lysis buffer at room temperature, for the WBCs analyses. Td tomato expression in WBC was assessed using the following antibodies: violetFluor™ 450 CD3 (clone 17A2, Tonbo Biosciences), CD45R/B220-FITC (clone RA3-6B2, BioLegend), Ly-6G, Gr1 PE-Cyanine7 (clone RB6-8C₅, BD Biosciences), for detection of T, B cells and Granulocytes, respectively, while CD45.2 PerCP-Cyanine5.5 (clone 104, eBioscience), CD45.1 APC (clone A20, BioLegend) antibodies were used to discriminate proportion of donor versus recipient chimerism, respectively. Bone marrow samples obtained after crashing were treated with ACK lysis buffer prior to analyses to remove RBC. The following biotinylated antibodies were used to discriminate lineage committed cells: CD45R/CD89/CD127/CD4/Ly6G-Ly6C/Ter119 (e Biosciences). An APC eFluor 780 conjugated streptavidin antibody (Invitrogen) was used to bind lineage committed cells pre-incubated with biotinylated Abs. To discriminate LSK cells a CD117-APC Ab (clone 2B8, Invitrogen) and a Ly6A/E (Scal)-PE-Cyanine7 Ab (clone D7, e Bioscience) were utilized. Peripheral blood samples were acquired on a CytoFLEX S analyzer (4 lasers: 405, 488, 561, 638 nm; 13 colors, Beckman Coulter), while bone marrow cells were acquired on a CytoFLEX LX analyzer (6 lasers: 375, 405, 488, 561, 638, 808 nm; 21 colors, Beckman Coulter). All acquisition data was analyzed using FlowJo software (Tree Star Inc).

The Experimental Results are now described

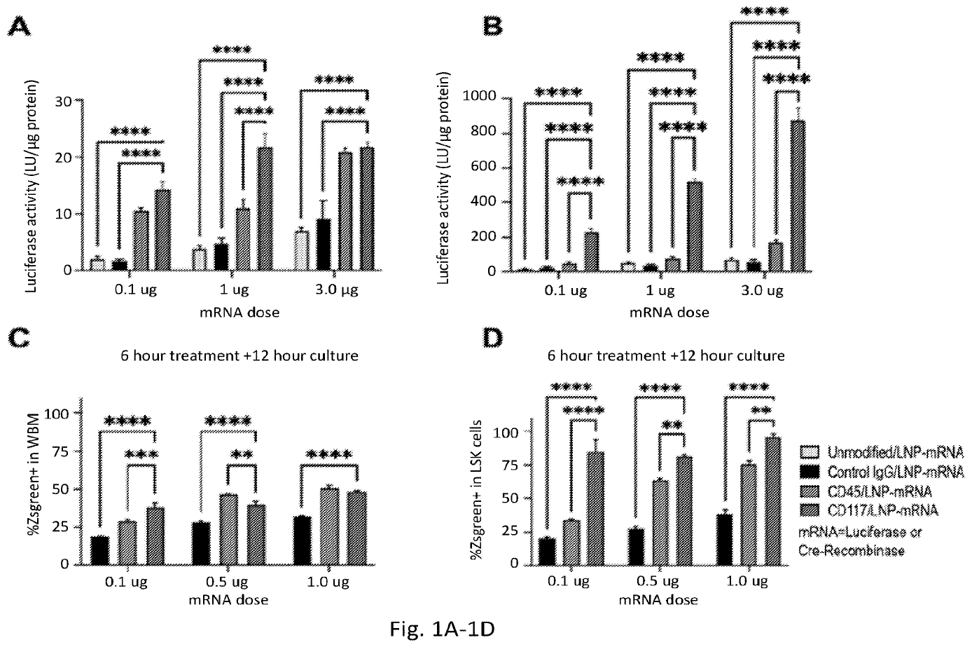

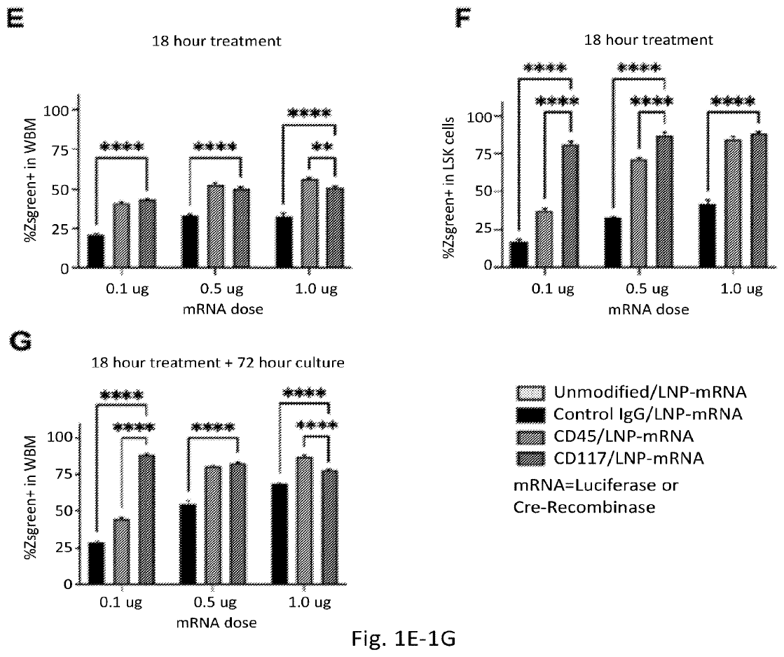

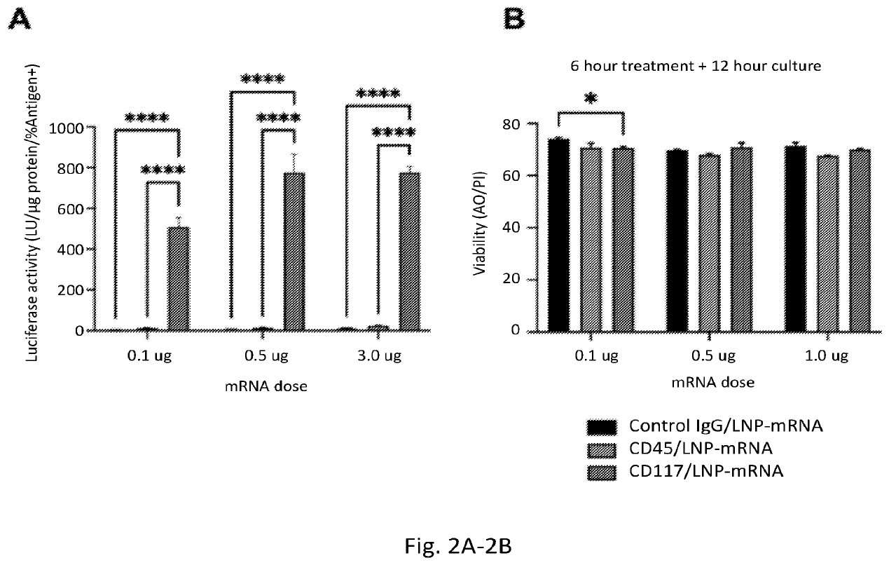

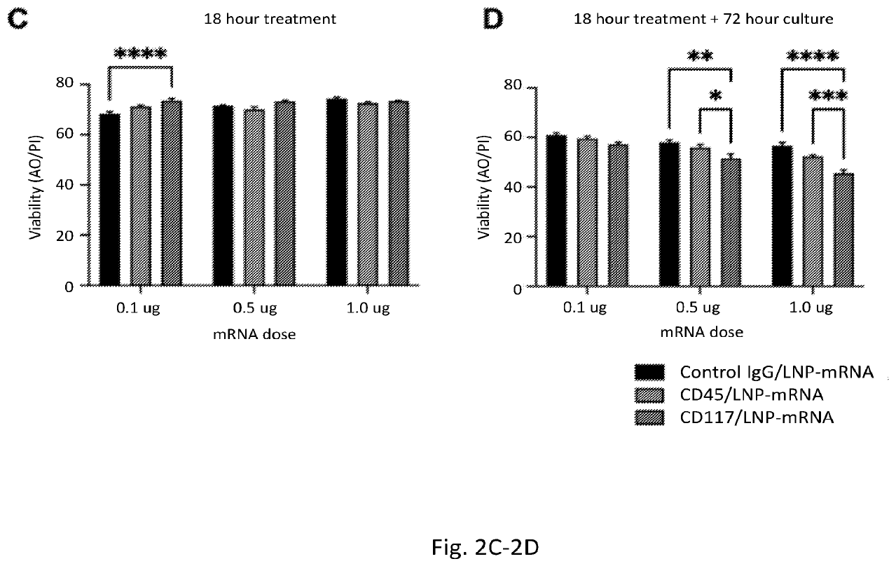

C57BL/6 whole BM (WBM) or lineage depleted BM cells (Lin−) was incubated in vitro with either unconjugated LNP encapsulating 0.1, 1, or 3 μg of nucleoside-modified luciferase mRNA (unmodified LNP-Luc), anti-CD45-conjugated LNP (CD45/LNP-Luc), anti-CD117-conjugated LNP (CD117/LNP-Luc), or isotype control IgG-conjugated LNP (control IgG/LNP-Luc). CD45/LNP and CD117/LNP were hypothesized to bind all hematopoietic-derived cells or stem and progenitor cells, respectively. Control IgG/LNP and unconjugated LNP were utilized as controls. The highest levels of luciferase activity in WBM were detected with CD117/LNP-Luc (FIG. 1A). Luciferase activity observed was further increased when Lin− cells were treated with CD117/LNP-Luc (FIG. 1B). Increased activity of CD117/LNP-Luc in Lin− cells was consistent with a 23-fold increase in the proportion of CD117+ in Lin− selected cells (2.8% CD117+ in WBM cells vs. 65% CD117+ in Lin-cells). CD117/LNP luciferase activity was 500 and 700-fold higher than CD45/LNP luciferase activity in WBM and Lin−, respectively, when normalized to the frequency of CD45 and CD117 positive cells in WBM and Lin− as measured by flow cytometry (FIG. 2A). Normalized luciferase activity suggests that CD117 mediated targeting and delivery is superior to CD45 mediated targeting in vitro. This demonstrated efficient targeting and functional delivery of mRNA with CD117/LNP.

CD117/LNP encapsulating Cre recombinase mRNA (CD117/LNP-Cre) was used to test LNP-mediated genetic recombination in HSCs and persistence of the editing in conjunction with three reporter murine models. These murine models (Ai6, Ai9 and Ai14) are engineered with a Cre-responsive reporter allele comprised of a loxP-flanked STOP cassette preventing transcription of a CAG promoter-driven green or red fluorescent reporter gene (ZsGreen1 for Ai6 and tdTomato for Ai9 and Ai14, respectively) inserted into the Gt (ROSA) 26Sor locus (14). The fraction of edited WBM cells (FIG. 1C), and the subset of edited Lin−Scal+cKit+ (LSK) within the BM (FIG. 1D), exhibited a dose dependency (0.1 to 1 μg mRNA) when incubated with CD45/LNP-Cre and control IgG/LNP-Cre. The majority of LNP mediated transfection occurred within 6 hours (FIGS. 1C and 1D), compared to 18 hours of treatment (FIGS. 1E and 1F). Targeting rates in the LSK subset of WBM cells were consistently significantly higher with CD117/LNP-Cre than with CD45/LNP-Cre or Control IgG/LNP-Cre, suggesting saturation of cKit+ cells by CD117/LNP-Cre at the lowest dose tested (FIGS. 1C and 1D). CD117/LNP-Cre showed higher efficacy in LSK cells at lower concentrations: treatment with 0.1 μg CD117/LNP-Cre was 2.5-fold more effective at targeting LSK cells compared to treatment with CD45/LNP-Cre (FIG. 1D). There was no significant difference between targeted cell frequency in the LSK subset between the 0.1 μg and 0.5 μg dose or 0.5 μg and 1 μg dose (p>0.05, unpaired, two-sided t-test). Media of cells treated for 18 h with LNP-Cre was replaced and they were kept for 3 additional days in culture to assess the maximum targeting achieved after exposing WBM to LNP. The rate of targeted cells increased over 3 days without additional LNP exposure (FIG. 1G): at a dose of 0.1 μg CD117/LNP-Cre, 88.5% WBM cells were tdTomato+ vs. 43.5% at 18 h (FIG. 1G and FIG. 1E), indicating additional mRNA translation, cre-mediated recombination, and tdTomato transcription/translation occurred beyond the 18 h LNP exposure. Importantly, LNP-Cre treatment had no effect on cell viability across formulations, regardless of the targeting antibody (FIG. 2B-D). Hence, it was determined that the use of CD117/LNP-Cre was superior to that of CD45/LNP-Cre to modify HSCs and CD117-LNP-Cre was selected for subsequent experiments.

### Anti-CD117 LNPs Edit Multipotent and Self-Renewing Long-Term HSCs Ex Vivo

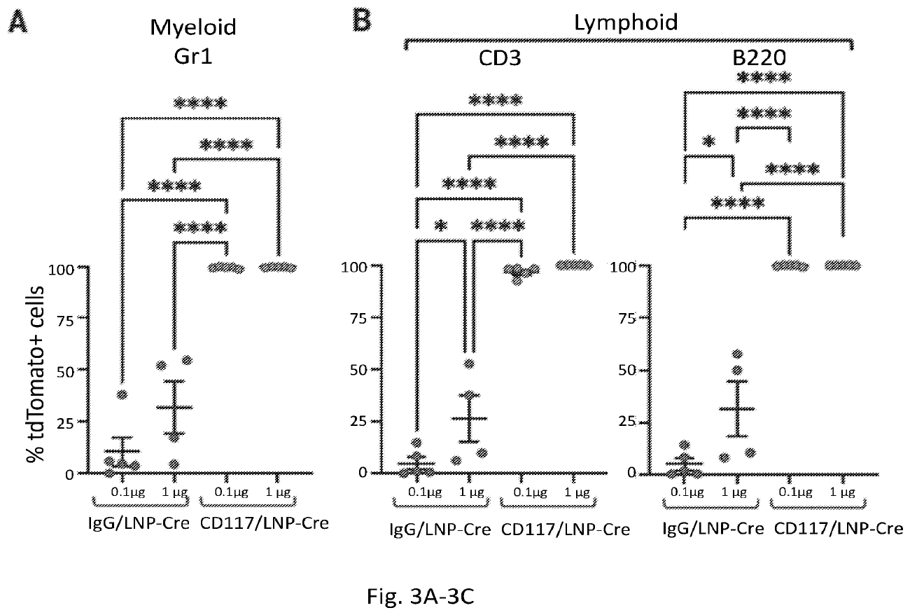

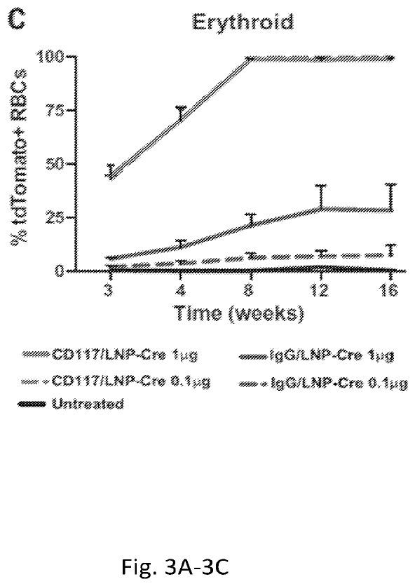

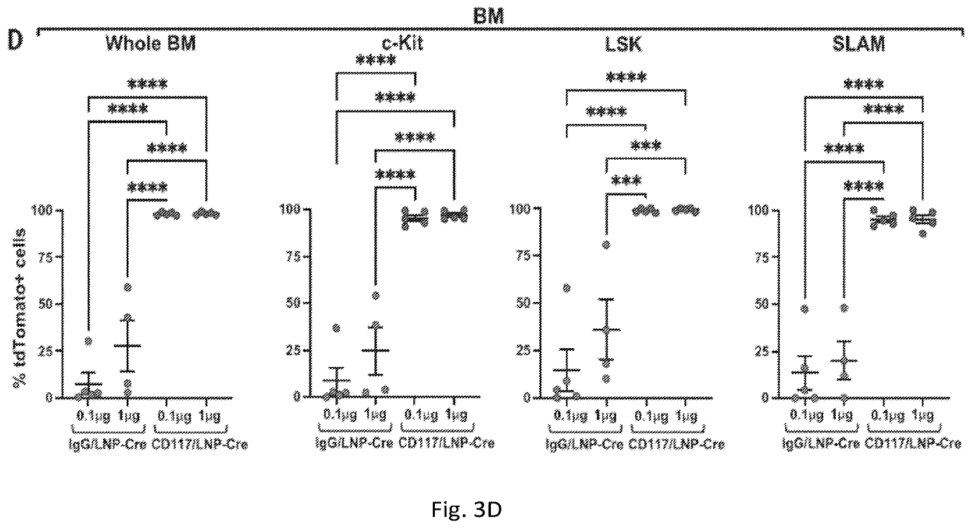

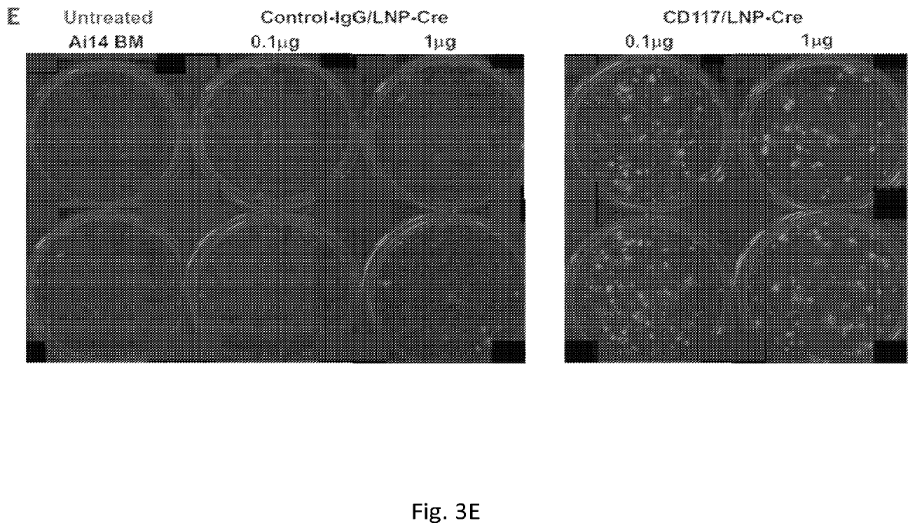

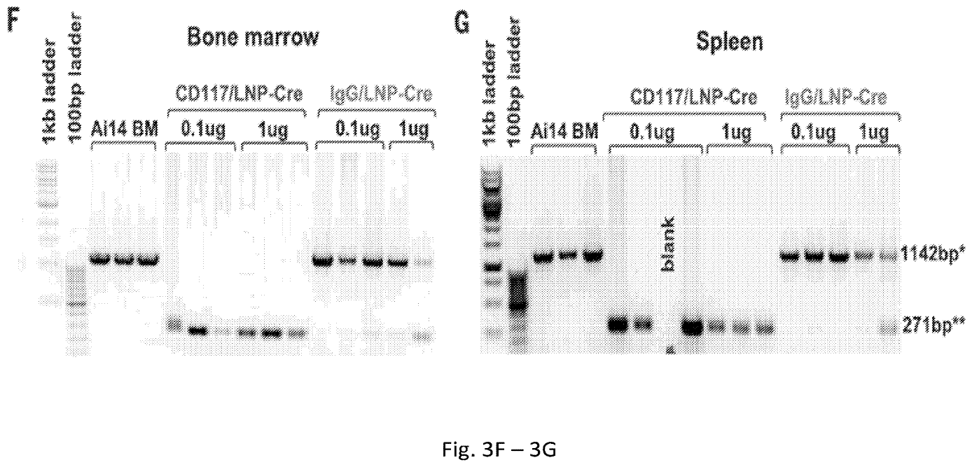

To evaluate multipotency in cells edited by use of CD117/LNP-Cre, lethally irradiated congenic C57BL/6 CD45.1-recipient mice were transplanted with Ai14 BM cells treated ex vivo with increasing doses of CD117/LNP-Cre and control IgG/LNP-Cre. Because HSCs give rise to all blood cell lineages, reporter gene expression was followed in peripheral blood cells over time and analyzed the BM at the 4-month endpoint (FIG. 3A through FIG. 3G). The percentage of CD117/LNP-Cre-mediated tdTomato-positive Ai14 erythroid cells in recipient mice increased with time after HSCT, which is consistent with the engraftment of donor HSC (FIG. 3C). Mice had durable editing in all lineages, specifically myeloid cells (Gr1+, FIG. 3A), lymphoid cells (CD3+ and B220+, FIG. 3B), and erythroid cells (FIG. 3C) at 4 months after HSCT, which is consistent with genome editing of multipotent HSCs.

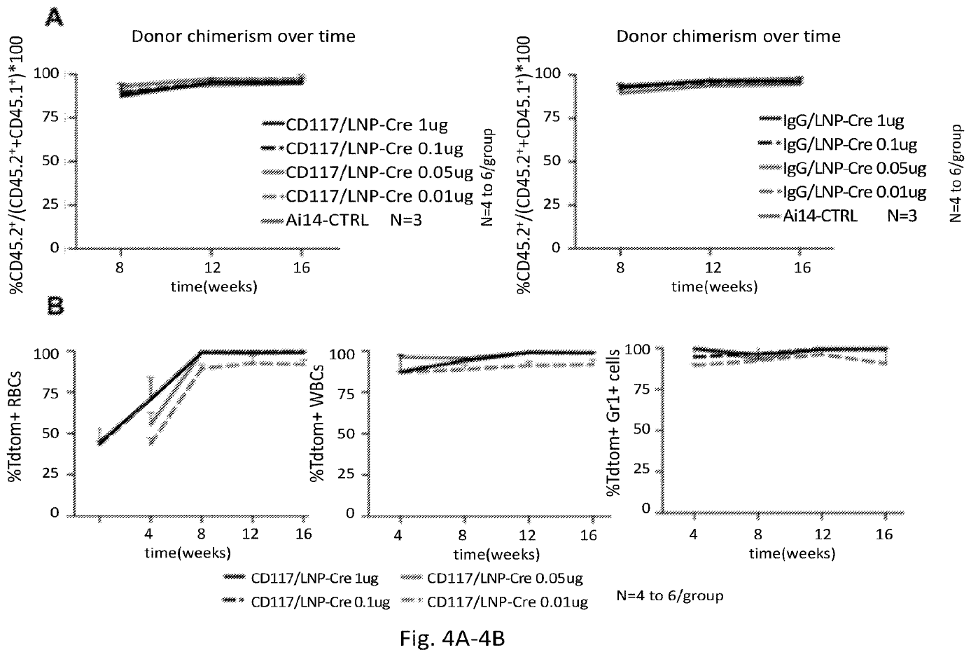

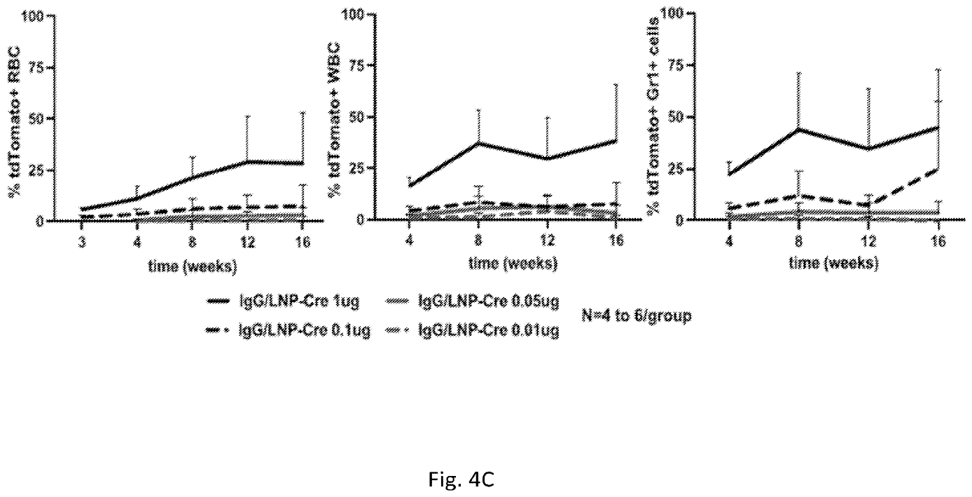

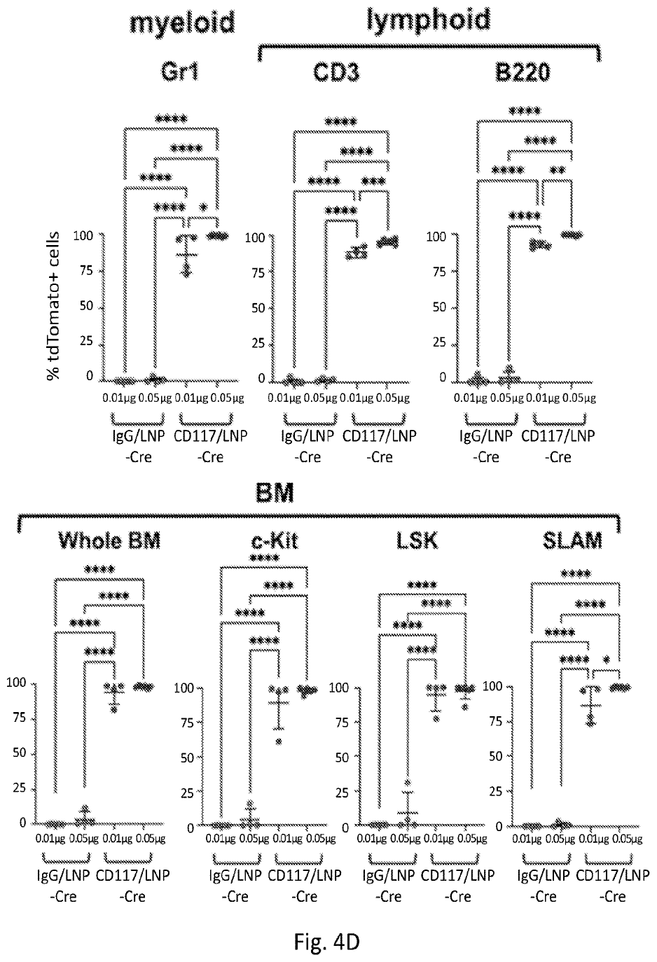

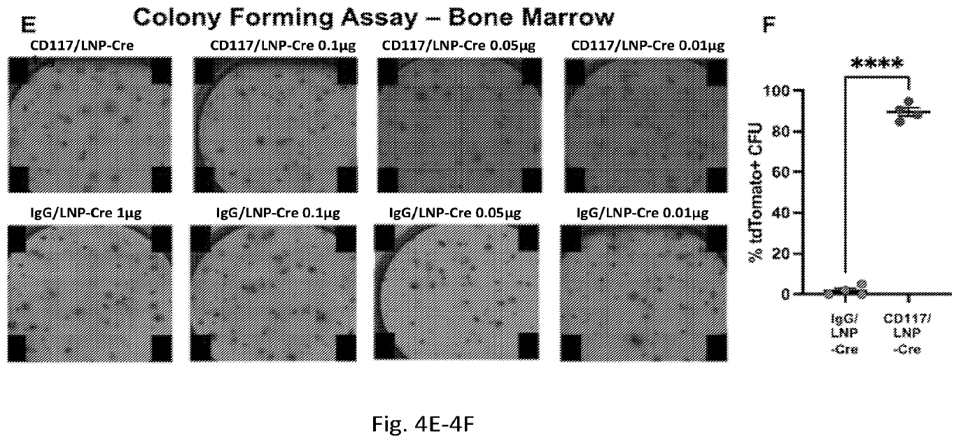

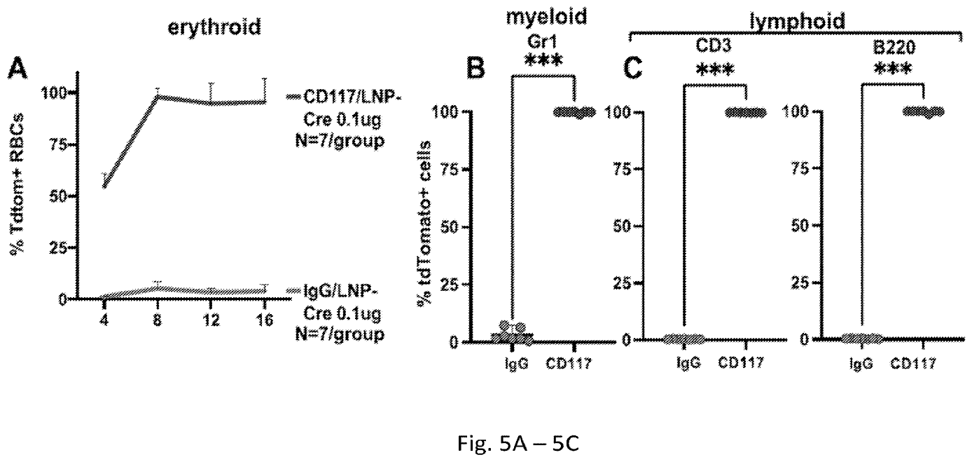

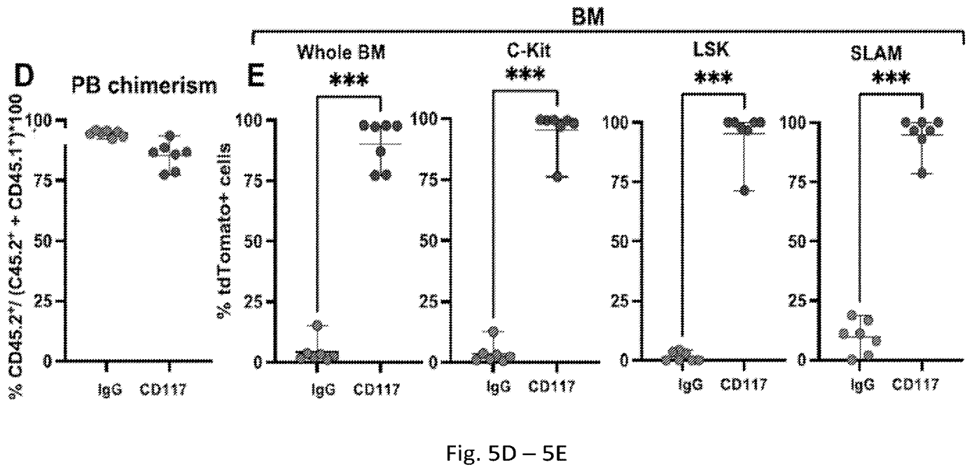

Editing rates in long-term HSCs (LT-HSC, LSK CD150+CD48−, aka SLAM) was 95% at the 0.1- and 1-mg mRNA dose with CD117/LNP-Cre compared with 13.5 and 20% with control IgG/LNP-Cre, respectively (FIG. 3D), which was similar to that seen in the WBM, the c-Kit+, and the LSK cell subsets. Donor chimerism was consistently high among all groups (>94% at 4 months) (FIG. 4A). The gene-editing rates of ex vivo-treated BM cells were dose dependent (FIG. 4B and FIG. 4C). Red blood cell (RBC)- and leukocyte-editing rates with CD117/LNP-Cre were ≥99% at 0.05-, 0.1-, and 1-mgmRNA doses and 91.8% at the 0.01-mg dose (FIG. 3A to FIG. 3C, and FIG. 4B to FIG. 4D). By comparison, targeting mediated by control IgG/LNP-Cre was near 0% at 0.01 mg (FIG. 4C and FIG. 4D). tdTomato+ Gr1+ cells had the fastest rise (FIG. 4B and FIG. 4C), which is expected given their rapid turnover of 2 to 3 days. BM cells harvested from these animals showed similar editing rates in colony-forming assays, a functional assay for clonogenic potential, and thus corroborated the flow cytometry results of LT-HSCs (FIG. 3E and FIG. 4E and FIG. 4F). At 4 months after HSCT, splenocytes had genome-editing levels comparable with those in the WBM (FIG. 3F, FIG. 3G), which is consistent with the migration of edited BM-derived cells to the spleen. To assess the stem cell potential of ex vivo-edited BM cells, secondary transplants were performed using the BM from two primary chimeras that were recipients of Ai14 BM cells treated ex vivo with either CD117/LNP-Cre or control IgG/LNP-Cre (0.1-mg dose of mRNA). Editing levels in secondary chimeras phenocopied those observed in the primary transplantation, which included sustained editing in the LT-HSC subset and editing in multiple hematopoietic lineages (FIG. 5A through FIG. 5E).

### In Vivo Editing of Multipotent and Self-Renewing Long-Term HSCs

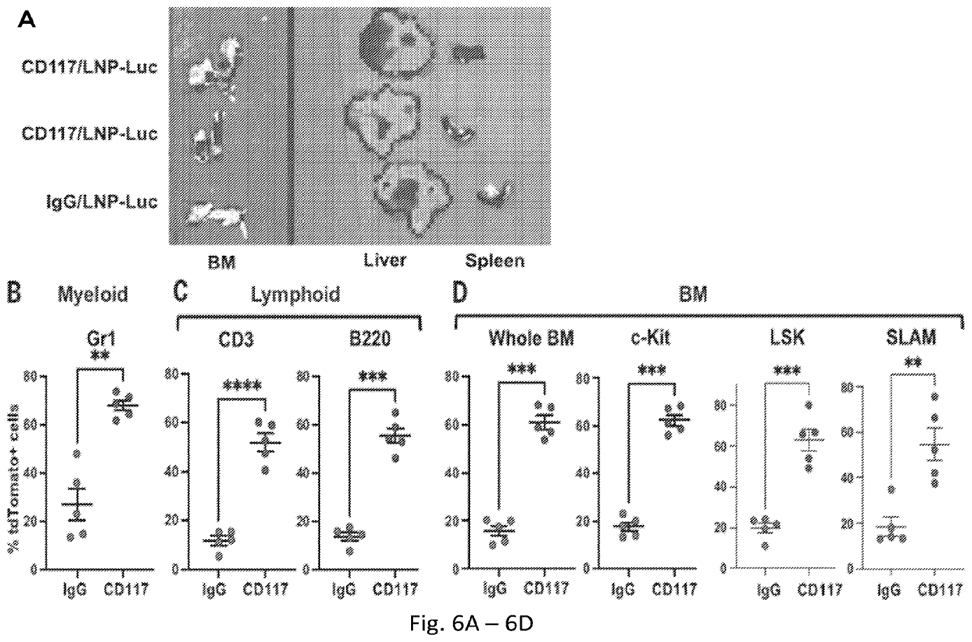

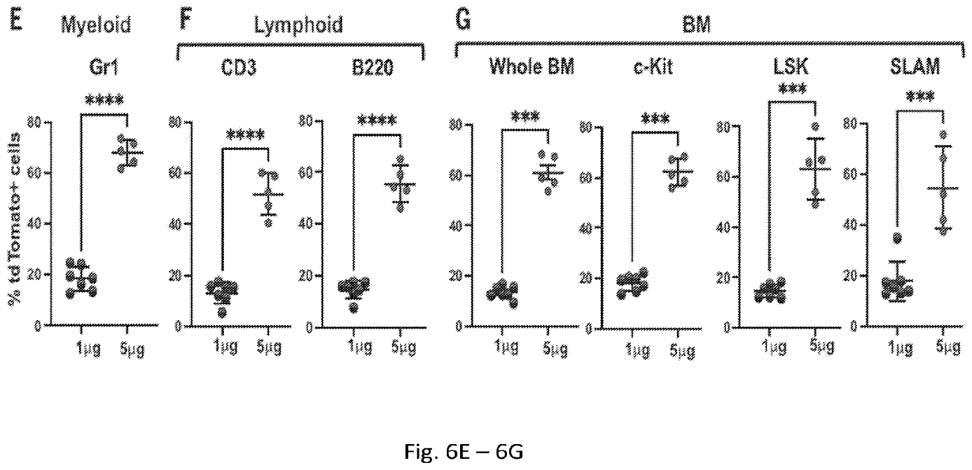

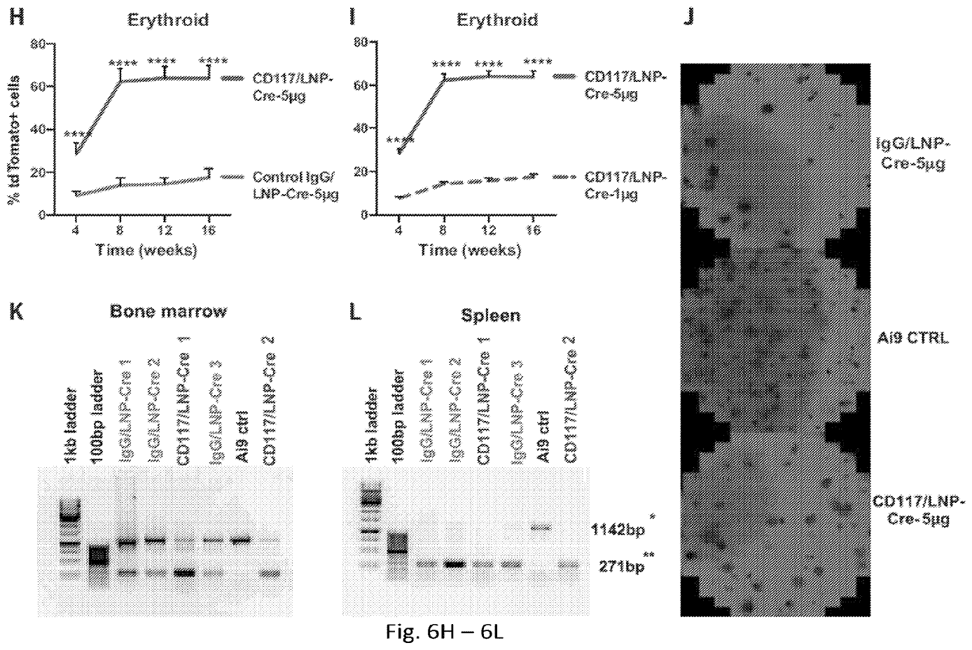

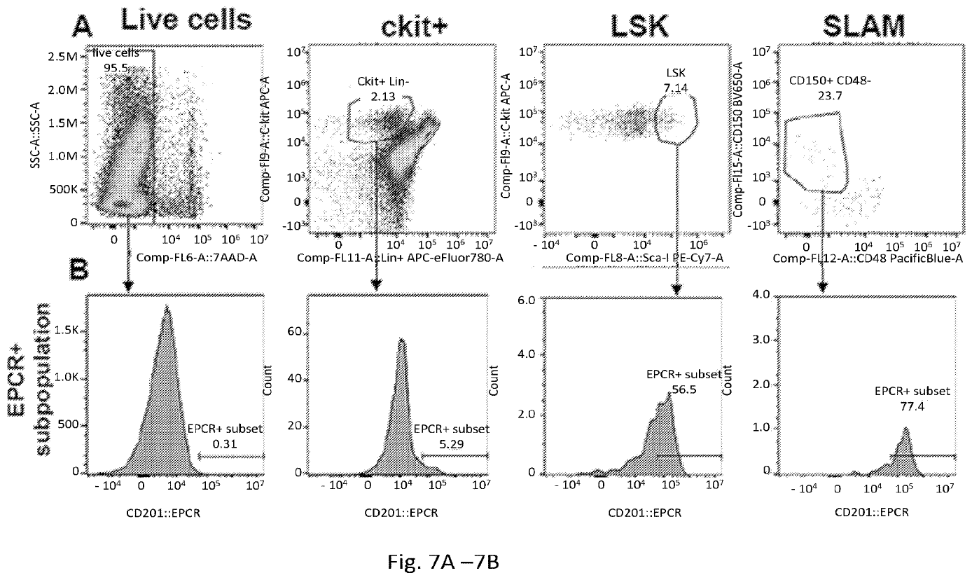

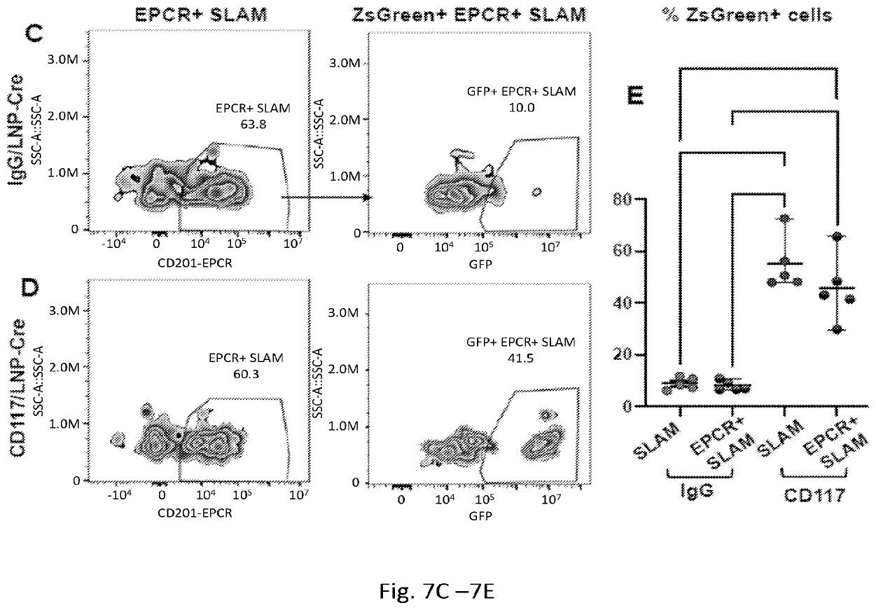

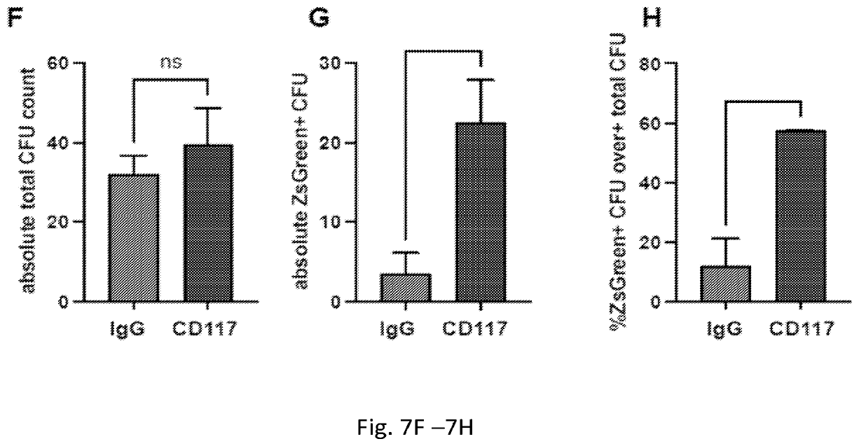

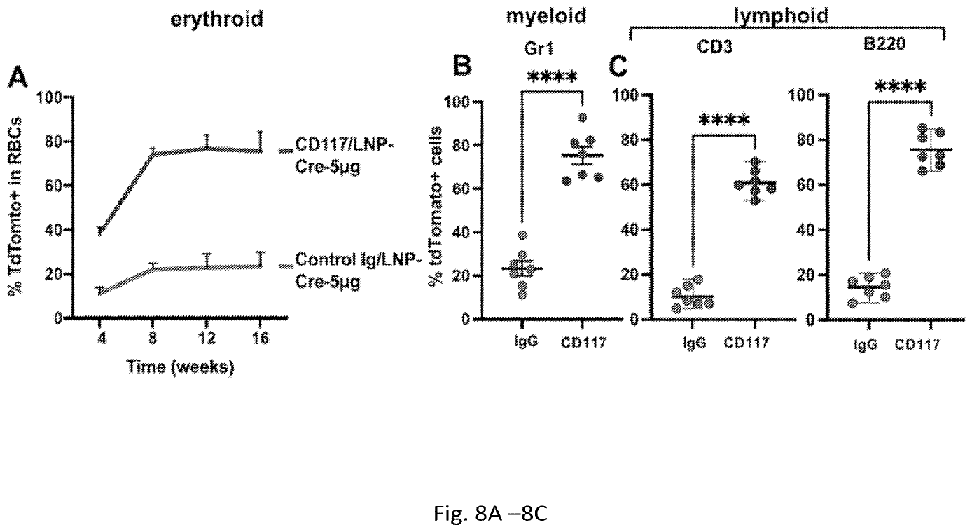

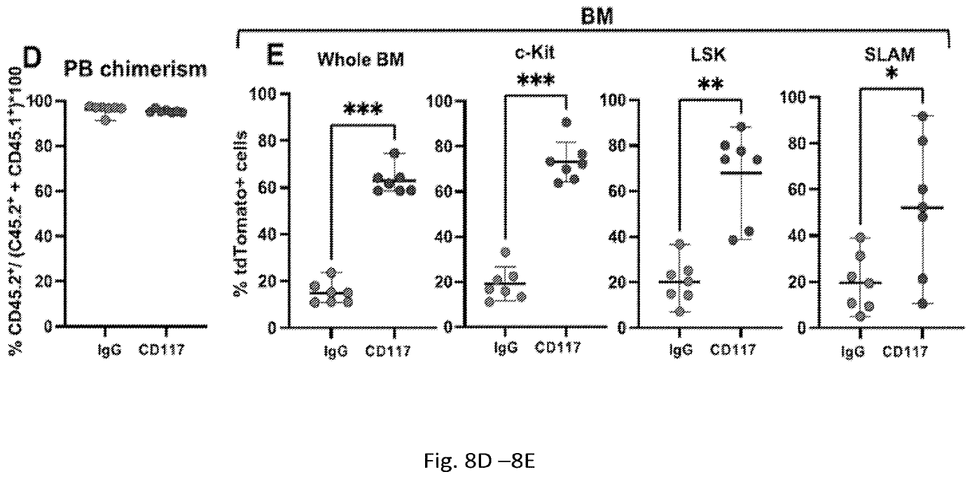

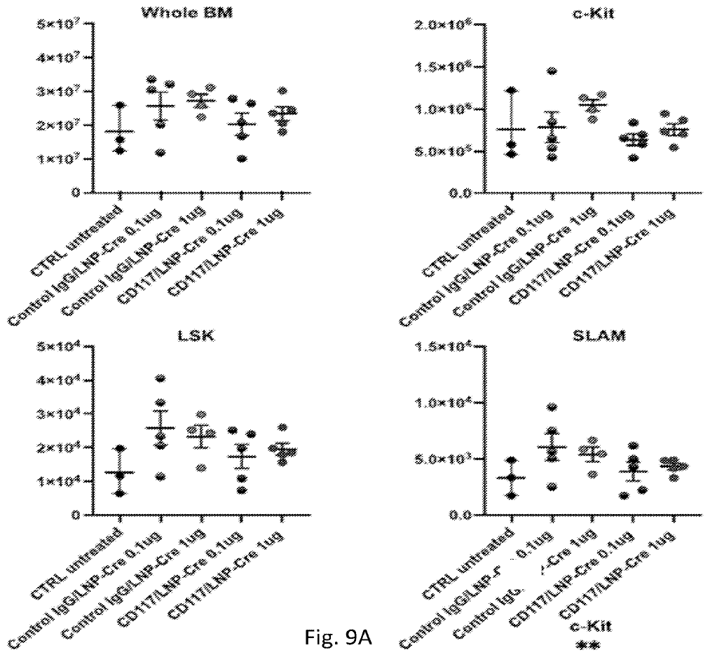

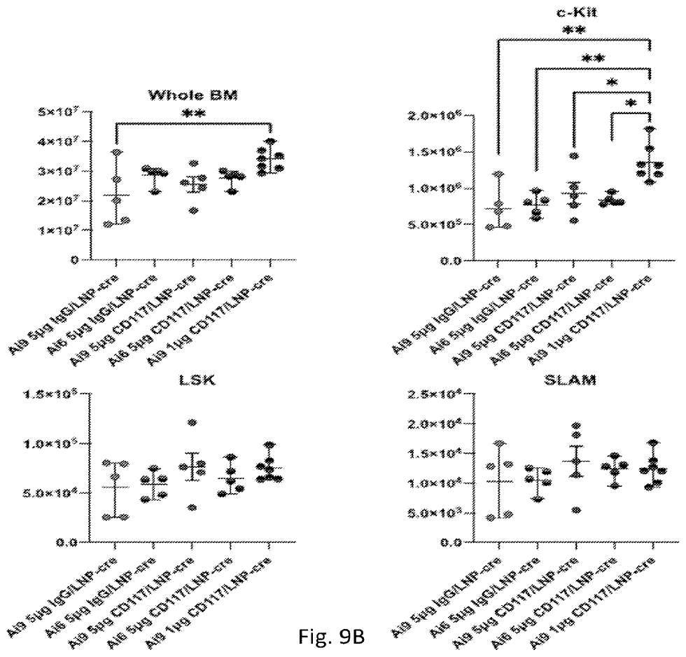

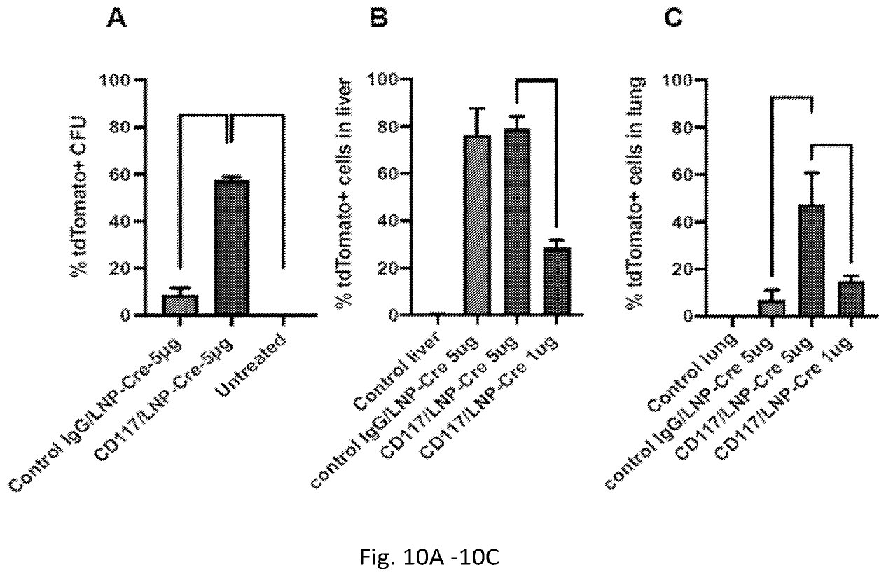

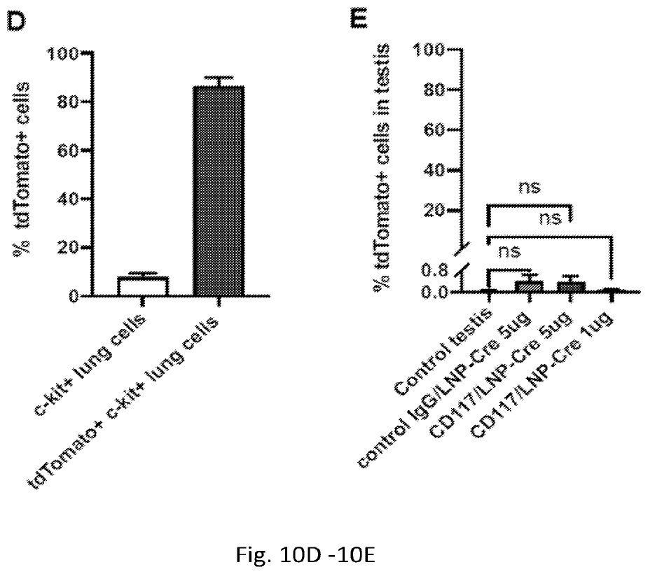

Given the near complete targeting of LT-HSC ex vivo with CD117/LNP-Cre, and the ability to target lung endothelial and T cells in vivo (Parhiz et al., 2018, J Control Release, 291:106-115; Marcos-Contreras et al., 2020, Proc Natl Acad Sci USA, 117:3405-3414; Tombacz et al., 2021, Mol Ther, 29:3293-3304; Rurik et al., 2022, Science, 375:91-96), without being bound by theory, it was hypothesized that LT-HSC could be targeted in vivo as well. Intravenous (i.v.) administration of CD117/LNP-luc, generated luciferase activity in the femur at 24 hours, whereas IgG/LNP-Luc did not (FIG. 6A). Both Control IgG/LNP-Luc and CD117/LNP-Luc showed comparable luciferase activity in the liver, as LNP bind ApoE and are non-specifically targeted to the LDL receptor, which is expressed on hepatocytes (Akinc et al., 2010, Mol Ther, 18:1357-1364). In vivo multilineage editing was tested by quantifying tdTomato expression in peripheral blood cells of intravenously IgG/or CD117/LNP-Cre-treated animals over time (up to 4 months) and tdTomato expression in BM, and specifically the LT-HSCs, at 4 months. At the same dose (5 mg), CD117/LNP-Cre-treated mice had significantly higher editing in all peripheral blood lineages (FIG. 6B and FIG. 6C) and threefold more editing in LT-HSCs (55% versus 19%, respectively) compared with that observed in control IgG/LNP-Cre-treated mice (FIG. 6D). HSC editing after in vivo treatment with CD117/LNP-Cre was dose dependent in peripheral blood and BM at 16 weeks, with a 5.5-fold increase in the percentage of gene-edited LT-HSCs with 5 versus 1 mg (FIG. 6E to FIG. 6G). LNP-Cre in vivo editing led to the appearance of edited RBCs with kinetics similar to that of the transplantation of ex vivo-treated BM (FIG. 6H and FIG. 6I). At 4 months after treatment with CD117/LNP-Cre, marking of HSCs was confirmed with visual inspection of tdTomato+ colony-forming units (CFUs) (FIG. 6J and FIG. 10A), and Cre-mediated genomic deletion in the BM and splenic DNAs was confirmed with polymerase chain reaction (PCR) (FIG. 6K and FIG. 6L). To further confirm in vivo LT-HSC targeting, editing of the endothelial protein C receptor (EPCR)+LT-HSC SLAM subpopulation (Balazs et al., 2006, Blood 107, 2317-2321), whose self-renewal properties are enriched compared with that of the LT-HSC SLAM population (Kent et al., 2009, Blood 113, 6342-6350), was investigated using the Ai6 model (FIG. 7A through FIG. 7H). Editing rates in the SLAM LT-HSC population and the EPCR+LT-HSC subpopulation were comparable within each cohort (CD117/LNP-Cre and Control IgG/LNP-Cre) (FIG. 7E). Mice injected with CD117/LNP-Cre bad 55%+10% edited SLAM LT-HSCs versus 46%+14% edited EPCR+LT-HSCs, whereas mice in the control group had 9%+2.3% edited SLAM LT-HSCs versus 8%+1.9% edited EPCR+SLAM LT-HSCs (FIG. 7E). CFUs from the BM of primary chimeras generated from in vivo-treated donors confirmed the editing differences between the two cohorts and yielded no difference in the number of colonies (FIG. 7F to FIG. 7H). To demonstrate that LNP-mediated editing targeted bona fide HSCs, chimeras from the initial in vivo experiment (Ai9 strain) were generated by transplanting irradiated congenic (C57BL/6 CD45.1) recipients with BM from mice 4 months after in vivo treatment with a 5-mg dose of CD117 or control IgG/LNP-Cre. Assessment of the hematopoietic-derived lineages, which included LT-HSCs in the BM, in these chimeras recapitulated editing found in the donor cells (FIG. 8A through FIG. 8E). LT-HSC editing in secondary chimeras was 52% for those derived from the CD117/LNP-Cre-treated primary and 19% for those derived from the control IgG/LNP Cre-treated primary. The absolute count of viable LT-HSCs was comparable among cohorts in both primary ex vivo transplants and in mice injected in vivo (FIG. 9A and FIG. 9B).

Nonhematopoietic Targeting after Targeted LNP Treatment

To quantify non-specific cellular uptake, tdTomato expression levels were compared in lung and liver cells at 4 month after in vivo treatment with a single dose of CD117/LNP-Cre (1 and 5 μg dose) or control IgG/LNP-Cre (5 μg dose). At 5 μg, liver editing was high (76-79% of cells), and editing was comparable between the two treatments (FIG. 10B), consistent with known non-specific ApoE and LDL receptor axis mediated LNP mediated uptake (Akinc et al., 2010, Mol Ther., 18 (7): 1357-1364). In the lung, tdTomato expression mediated by CD117/LNP-Cre delivery was significantly higher (7-fold) than that of mice injected with control IgG/LNP-Cre (FIG. 10C). Editing observed in the perfused lung was 3-fold higher with 5 μg of CD117/LNP-Cre compared to 1 μg. This effect was partly “on-target” editing: ~8% of lung cells were cKit+, and ~90% of cKit+ cells were edited (FIG. 10D). Cells collected from the testis were also analyzed and did not show significant variations from baseline levels in control mice (FIG. 10E). Additionally, none of 50 offspring sired by male mice treated with CD117/LNP-Cre in vivo (n=4) or 39 offspring sired by male mice treated with control IgG/LNP-Cre (n=3) in vivo expressed tdTomato.

Efficient In Vitro Editing of Primary Sickle Cell Disease Hematopoietic Stem and Progenitor Cells with Anti-Human CD117

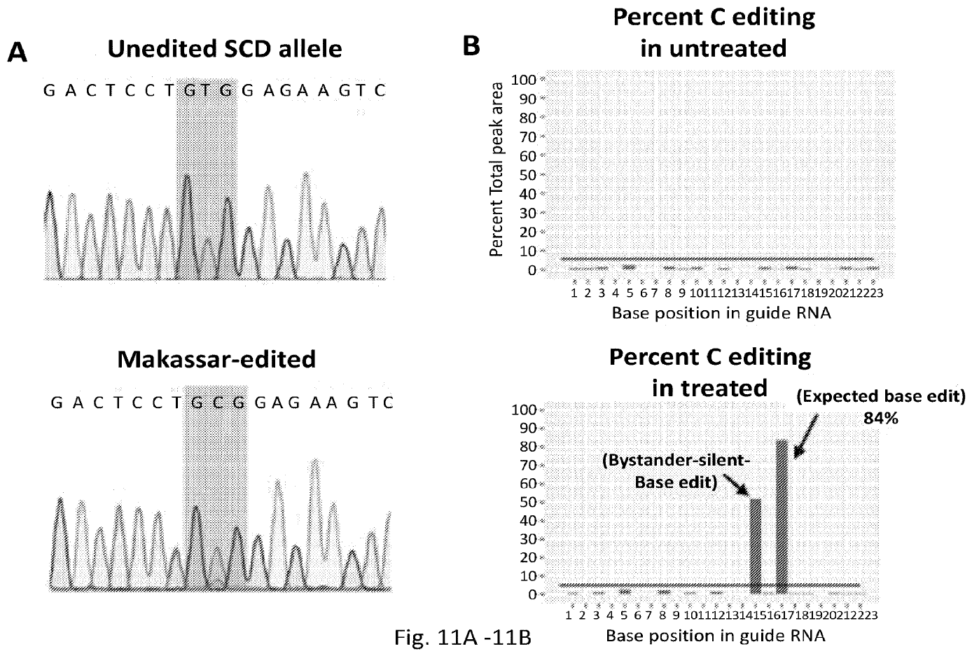

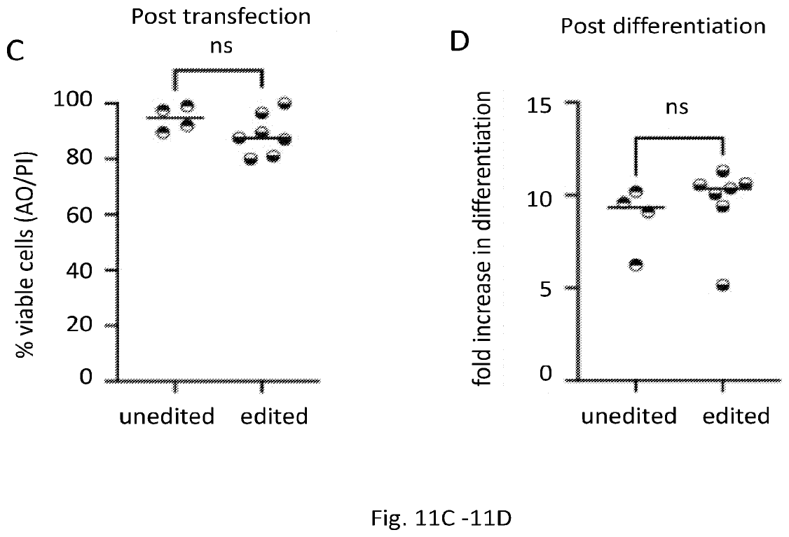

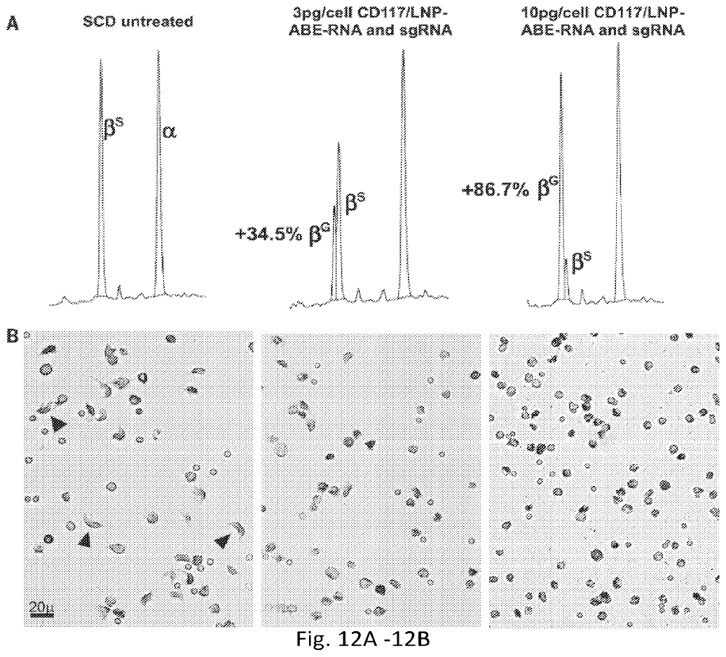

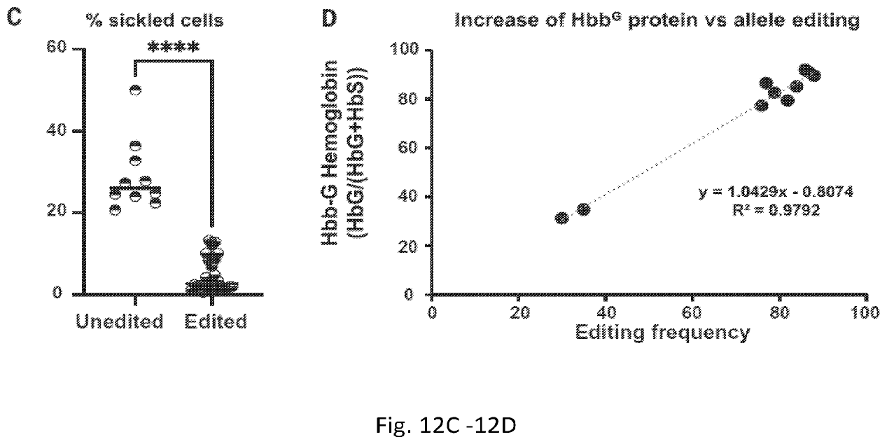

To assess the feasibility of using this platform for therapeutic human genome editing, the targeting was adapted to human CD117 and LNPs that contained mRNA and encoded a Cas9 adenine base editor (ABE) fusion and LNPs that carried a single-guide RNA (sgRNA) targeted to the b-globin sickle cell mutation were used. Adenine base editing of the A to G leads to conversion of the pathogenic E6V (HBBS) mutation to a nonpathogenic E6A variant (HBBG-Makassar) (Newby et al., Nature 595, 295-302). This therapeutic strategy was applied to convert pathogenic sickle hemoglobin (HBBS) to nonpathogenic G-Makassar hemoglobin (HBBG) on four sickle cell specimens from separate donors (FIG. 11A and FIG. 11B). A molecular excess of sgRNA to ABE mRNA-containing LNPs led to efficient editing with the highest rates (88%) at 10 μg/cell dose (FIG. 12A). This led to a corresponding increase in HBBG protein (up to 91.7% of b-like globin) and HBBS decrease after in vitro erythroid differentiation, as well as a nearly complete absence of sickled cells upon exposure of the erythroblasts to hypoxic conditions (FIG. 12B and FIG. 12C). Editing levels and the increase of HBBG were directly correlated (FIG. 12D). LNP doses from 3 μg/cell up to 10 μg/cell did not alter the viability and proliferation rate of erythroid progenitor cells in vitro (FIG. 11C and FIG. 11D).

PUMA mRNA Depletes HSCs from Mouse BM In Vitro

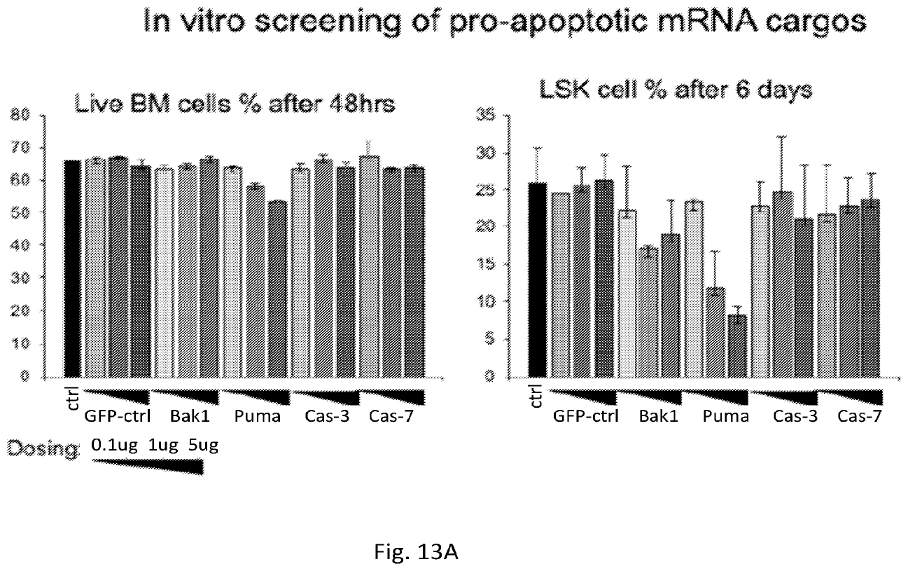

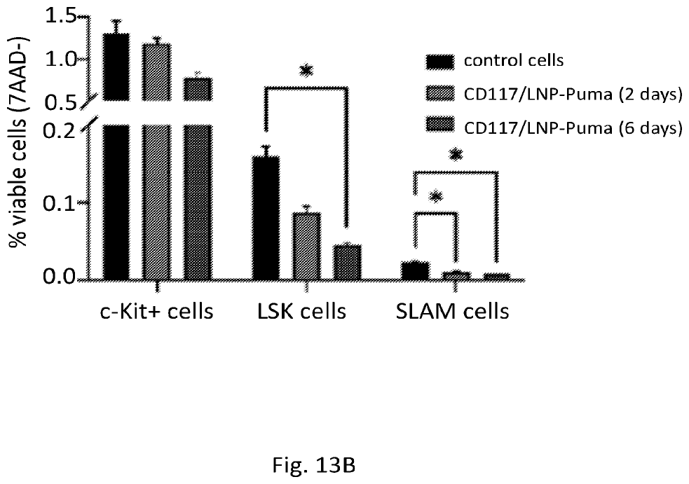

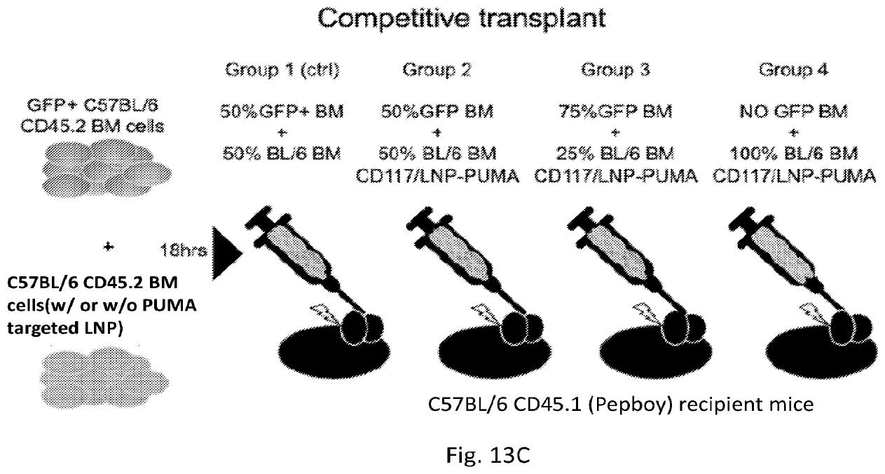

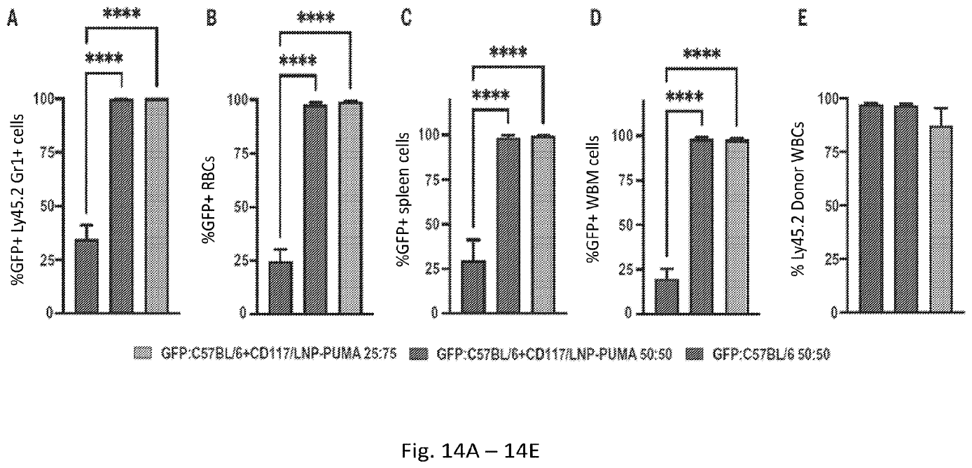

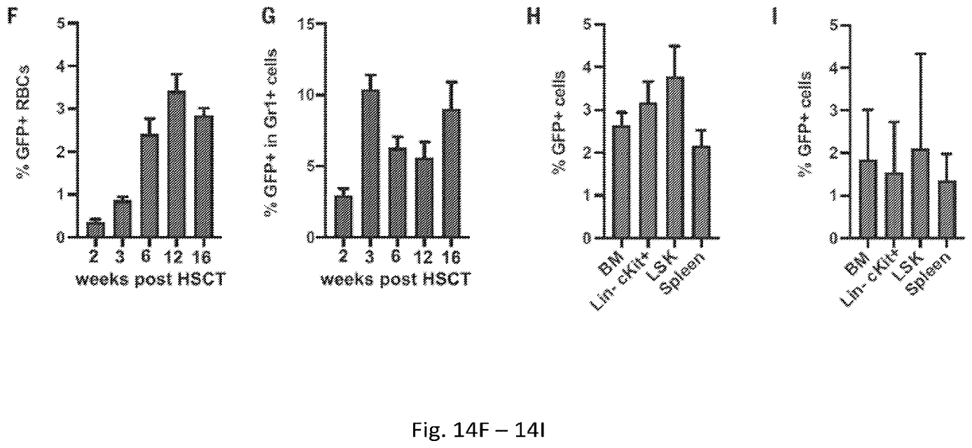

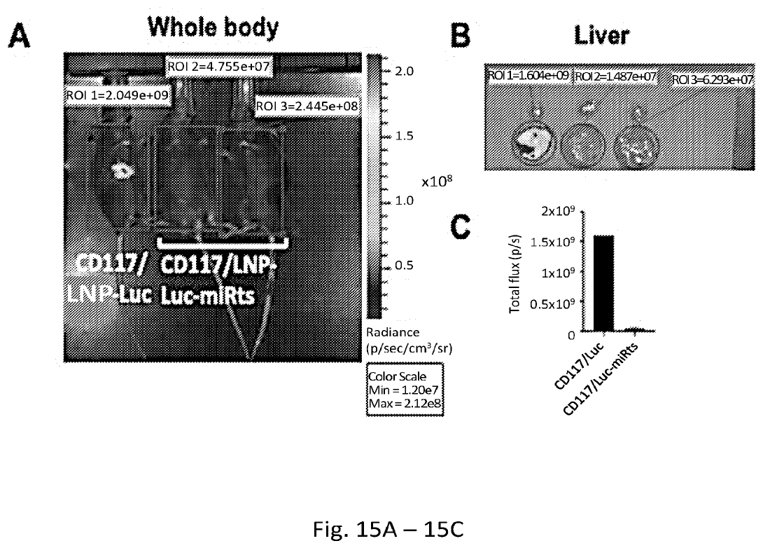

The survival of human and mouse HSCs depends on the anti-apoptotic geneMcl-1 (Opferman et al., 2005, Science 307, 1101-1104; Campbell et al., 2010, Blood 116, 1433-1442); thus, the ability of CD117/LNP to deplete BM cells using pro-apoptotic mRNA was tested. A variety of pro-apoptotic mRNAs were tested that act within this pathway. Among those genes tested on mouse C57BL/6 BM cells, treatment with PUMA mRNA reduced BM and LSK viability after 48 hours and 6 days in culture, respectively (FIG. 13A). To confirm that LNP-PUMA mRNA treatment depleted multilineage hematopoietic stem and progenitor cell (HSPCs), competitive HSCT was performed in which C57BL/6 CD45.2 BM was treated with CD117/LNP-PUMA ex vivo (5 mg) and transplanted at equal or increasing ratios against untreated green fluorescent protein (GFP+) C57BL/6 CD45.2 BM cells into lethally irradiated congenic C57BL/6 CD45.1 recipients (FIG. 13C, schema). If CD117/LNP-PUMA efficiently depletes HSCs, mice receiving only CD117/LNP PUMA-treated BM (C57BL/6 CD45.2) would experience BM failure from the depletion of HSCs, and those receiving competitive BM would have an overrepresentation of untreated GFP+BM. The results were consistent with our expectations: Mice injected with only CD117/LNP-PUMA-treated GFP-BM cells died within 2 weeks from the HSCT, which indicates that HSCs were not viable and did not engraft. Mice who received 50 or 75% CD117/LNP-PUMA-treated GFP-BM had <0.5% donor GFP−Gr1+ cells or RBCs (FIG. 14A and FIG. 14B) at 4 months (endpoint) versus the expected 50 to 75%. The remainder of donor cells (CD45.2) were GFP+ (untreated) cells. This is consistent with the essentially complete depletion of engrafting, multilineage HSCs with ex vivo treatment of CD117/LNP-PUMA. By comparison, mice injected with control untreated GFP+/−C57BL/6 CD45.2 BM at a 1:1 ratio had 25% GFP+ cells (FIG. 14A to FIG. 14D). At endpoint, all groups had similar donor chimerism (>94% C57BL/6 CD45.2) (FIG. 14E).

HSC Depletion with CD117/LNP-PUMA Allows for BM Engraftment

HSC depletion in vivo was confirmed with intravenous injection of CD117/LNP-PUMA at 0.05 mg/kg in C57BL/6 mice, which showed a 71 and 58% decrease in the frequency of LSK cells and LT-HSCs in BM isolates 6 days after treatment, respectively (FIG. 13B). A 0.05 mg/kg mRNA dose was found to be the maximum tolerated dose. Animals treated with 0.15 mg/kg or more CD117/LNP-PUMA displayed decreased activity, elevations in the alanine transaminase/aspartate transaminase (AST/ALT) ratio, venous congestion of the lungs and liver, and mortality. In vivo CD117/LNP-PUMA HSC depletion as conditioning for HSCT was tested. After it was confirmed that a liver-specific microRNA (miRNA) binding site (mir-122) could decrease expression in the liver (Jain et al., 2018, Nucleic Acid Ther. 28, 285-296) (FIG. 15), liver-specific miRNA binding sites for mir-122 were incorporated into the 3′ untranslated region of our PUMA mRNA cargo.mir-122 is expressed in vertebrate hepatocytes and can decrease the expression of transgenes in hepatocytes. C57BL/6 recipients received 0.05 mg/kg mRNA CD117/LNP-PUMA miRNA intravenously 7 days before the infusion of 10×10⁶ GFP+C57BL/6 BM cells. The level of engraftment was evaluated after 2 weeks and up to 16 weeks (endpoint) and confirmed progressive increase and stabilization of GFP+Gr1+ cells and RBCs, as well as hematopoietic cells in the spleen (CD45+) and BM (FIG. 14F to FIG. 14H); 3.8% of BMLSK cells were donor. By comparison, C57BL/6-recipient mice not treated with CD117/LNP PUMA conditioning failed to engraft donor cells. Secondary transplantation of the cells that engrafted with PUMA conditioning phenocopied the donors (FIG. 14I). This shows that in vivo targeting with CD117/LNP-PUMA effectively depleted HSCs, which allowed GFP+BM cells to successfully engraft without need of chemotherapy or irradiation. These engraftment rates are consistent with those reported to be sufficient for the cure of SCID with healthy donor BM (Cavazzana, et al., 2016, Hum. Gene Ther. 27, 108-116; Cavazzana-Calvo et al., 2007, Blood 109, 4575-4581; Dvorak et al., 2019, Biol. Blood Marrow Transplant. 25, 1355-1362) and may overcome BM failure syndromes.

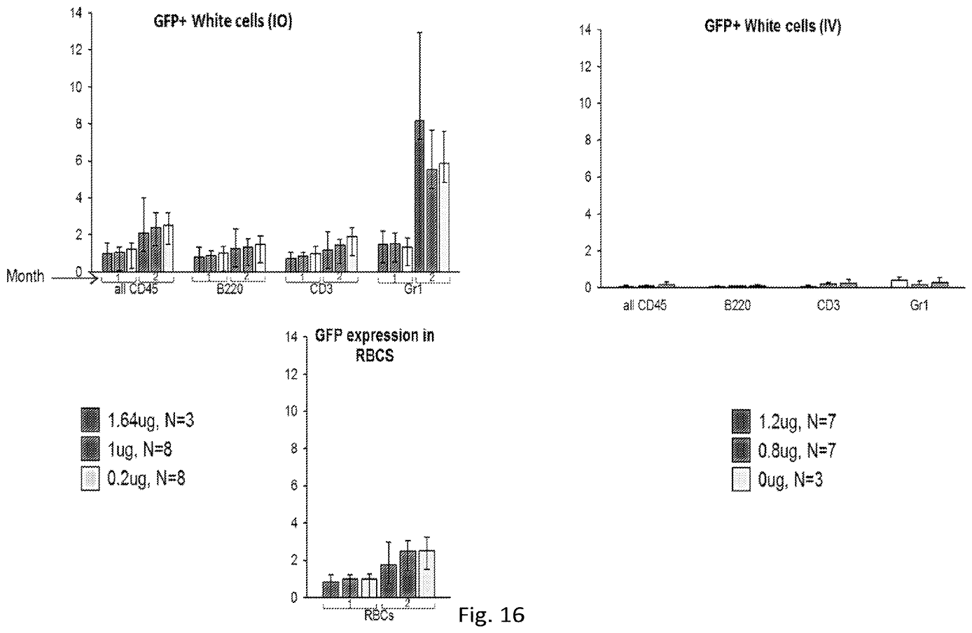

FIG. 16 demonstrates intravenous (IV) versus intraosseous infusion (IO) preconditioned mice.

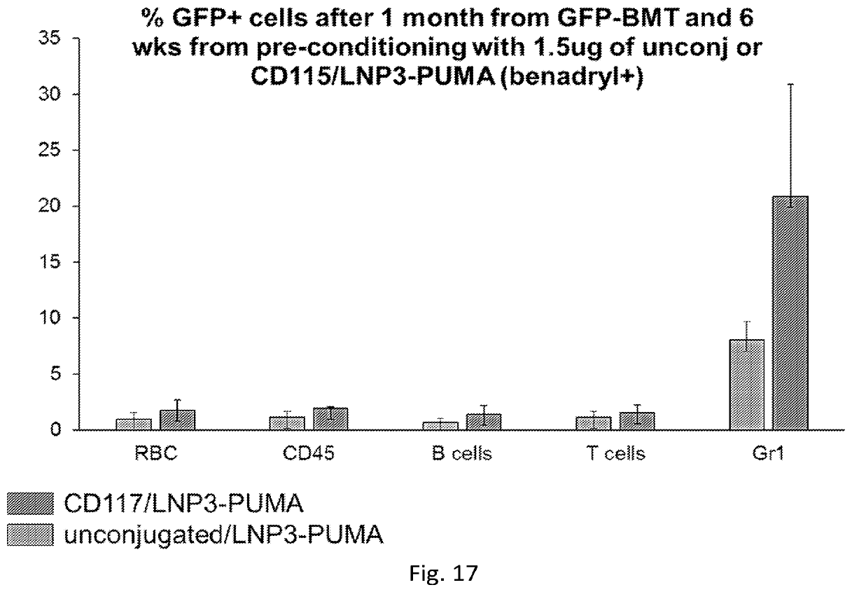

In recent preliminary experiments (FIG. 17), it was observed that intraperitoneal (IP) pre-treatment with Benadryl (20 mg/kg) allows for increased tolerated conditioning (0.075 mg/kg) with CD117/LNP-PUMA and increased level of engraftment at 1 month post HSCT (n=3, with 20.88%+10 of donor granulocytes). Benadryl was utilized on the assumption that mast cells express the CD117 receptor and, upon treatment with CD117/LNP-PUMA, release histamine, causing toxicity and interfering with engraftment of donor bone marrow cells. Benadryl acts as an inverse agonist at the H receptors, thereby reversing the effects of histamine which dilates blood vessels and constrict bronchial air passages, hampers neurological functions and induces inflammation reaction symptoms.

TABLE 1
Sequences

| SEQ ID NO | Description | Sequence |
|---|---|---|
| 2 | eGFP | ATGGTGTCTAAGGGCGAGGAATTGTTTACAGGTGTGGT |
| | | GCCCATCCTGGTGGAGCTTGATGGCGATGTAAATGGAC |
| | | ACAAATTCTCCGTTAGTGGGGAAGGCGAAGGGGATGCC |
| | | ACCTACGGTAAGCTTACGCTGAAATTCATCTGCACCAC |
| | | TGGTAAACTCCCCGTGCCATGGCCAACCCTGGTCACGA |
| | | CCCTTACTTATGGGGTGCAGTGTTTTTCAAGGTACCCCG |
| | | ACCATATGAAACAACATGATTTCTTCAAGTCCGCCATG |
| | | CCGGAGGGGTACGTCCAGGAGAGAACAATCTTTTTCAA |
| | | AGATGACGGGAACTACAAGACTCGCGCAGAAGTCAAG |
| | | TTTGAGGGAGACACTCTCGTAAACCGAATTGAACTGAA |
| | | AGGAATTGACTTTAAGGAAGACGGTAATATACTGGGCC |
| | | ACAAGCTGGAGTATAATTATAACAGCCATAATGTGTAT |
| | | ATCATGGCAGACAAGCAAAAGAACGGCATTAAAGTGA |
| | | ACTTCAAGATCCGTCACAATATCGAGGATGGCAGCGTC |
| | | CAGCTGGCTGACCACTACCAGCAGAACACACCTATTGG |
| | | AGACGGCCCAGTTTTACTACCTGACAACCACTATCTCA |
| | | GTACACAGAGCGCCCTCTCTAAGGACCCTAATGAAAAG |
| | | CGGGATCATATGGTTTTACTGGAGTTTGTCACAGCTGC |
| | | GGGAATAACCTTGGGCATGGATGAGTTGTACAAATGA |
| 3 | Luc2 | ATGGAAGACGCCAAGAACATTAAGAAGGGCCCTGCAC |
| | | CCTTCTACCCACTGGAAGACGGTACTGCAGGGGAGCAG |
| | | CTGCACAAGGCCATGAAGCGGTATGCCCTCGTTCCTGG |
| | | CACTATCGCCTTCACAGATGCCCACATCGAAGTAGATA |
| | | TCACCTATGCTGAGTACTTTGAGATGAGTGTGAGACTG |
| | | GCAGAGGCAATGAAACGTTATGGACTGAACACCAACC |
| | | ATAGAATCGTAGTGTGCTCTGAGAACAGCTTGCAGTTC |
| | | TTCATGCCTGTCTTAGGAGCACTGTTCATAGGCGTCGCC |
| | | GTGGCACCAGCCAATGACATTTACAATGAGAGGGAGCT |
| | | CCTGAATAGTATGGGCATAAGCCAGCCAACAGTGGTTT |
| | | TCGTTTCCAAGAAAGGGCTTCAAAAAATCCTGAATGTG |
| | | CAAAAGAAGCTCCCTATCATCCAGAAGATCATTATAAT |
| | | GGACTCAAAGACTGATTACCAGGGCTTCCAGTCCATGT |
| | | ATACATTTGTCACCAGCCACCTTCCACCAGGCTTCAATG |
| | | AATATGATTTCGTGCCTGAGTCATTTGACAGGGACAAA |
| | | ACAATTGCACTCATTATGAATTCTTCCGGGTCAACAGG |
| | | TTTGCCCAAGGGGGTGGCGCTACCACACAGAACGGCGT |
| | | GTGTGCGCTTTAGCCATGCTCGAGACCCCATCTTCGGG |
| | | AATCAGATCATTCCCGACACTGCCATCTTGTCTGTCGTC |
| | | CCTTTTCACCATGGTTTCGGTATGTTCACCACGTTGGGC |
| | | TACCTGATCTGTGGTTTCCGGGTAGTACTGATGTACAG |
| | | GTTTGAAGAAGAGCTCTTCCTGCGGAGCCTACAGGACT |
| | | ACAAGATCCAGAGCGCACTGCTGGTGCCCACCCTTTTT |
| | | TCGTTCTTTGCCAAATCCACCCTGATTGATAAATATGAC |
| | | CTATCCAACCTTCATGAGATAGCATCTGGAGGTGCTCC |
| | | TCTGAGTAAAGAAGTCGGAGAAGCTGTAGCCAAGAGG |
| | | TTCCACCTGCCAGGCATTCGCCAAGGATATGGCCTGAC |
| | | AGAGACTACAAGTGCCATTTTAATAACTCCAGAGGGAG |
| | | ATGACAAGCCTGGGGCTGTGGGCAAAGTTGTTCCGTTC |
| | | TTCGAAGCTAAGGTGGTTGACCTGGACACAGGAAAAAC |
| | | CCTGGGCGTCAACCAGCGTGGGGAACTCTGCGTCCGAG |
| | | GGCCCATGATCATGTCTGGCTACGTGAACAACCCCGAG |
| | | GCCACCAATGCCCTCATTGACAAGGATGGCTGGCTCCA |
| | | TTCAGGAGACATTGCCTACTGGGACGAGGATGAACACT |
| | | TTTTTATTGTGGACAGGCTCAAGTCGCTTATCAAGTACA |
| | | AAGGCTACCAGGTGGCTCCTGCTGAATTGGAATCCATC |
| | | TTACTTCAGCACCCCAACATATTTGATGCGGGTGTGGC |
| | | CGGTCTACCGGATGATGATGCAGGAGAGCTGCCCGCTG |
| | | CTGTTGTTGTGCTGGAGCATGGTAAGACCATGACTGAG |
| | | AAGGAGATTGTGGACTATGTAGCGTCTCAAGTCACGAC |
| | | CGCTAAAAAACTAAGAGGGGGTGTGGTCTTTGTGGATG |
| | | AGGTCCCAAAAGGATTGACTGGGAAGCTGGATGCTCGC |
| | | AAAATAAGAGAAATCCTCATCAAAGCAAAGAAGGGAG |
| | | GGAAAATTGCTGTCTGA |
| 4 | Cre | ATGTCTAATCTCCTCACTGTGCATCAGAATCTTCCAGCT |
| | | TTACCGGTAGACGCCACGTCTGATGAAGTGCGCAAAAA |
| | | TCTCATGGACATGTTCAGGGACCGGCAAGCCTTCAGTG |
| | | AGCACACATGGAAGATGTTGTTGTCTGTGTGTCGCTCCT |
| | | GGGCTGCCTGGTGCAAACTTAACAACAGGAAGTGGTTC |
| | | CCTGCAGAGCCTGAGGACGTCAGAGACTATCTGCTCTA |
| | | CTTGCAAGCACGAGGACTCGCGGTAAAGACCATCCAGC |
| | | AGCACCTGGGCCAGCTGAACATGCTGCACAGGAGGTCT |
| | | GGGCTGCCCCGACCAAGTGACTCAAATGCTGTGTCTCT |
| | | GGTCATGAGACGCATCCGCAAGGAGAATGTGGATGCC |
| | | GGAGAACGAGCCAAGCAGGCTCTGGCTTTTGAACGGAC |
| | | AGACTTTGATCAGGTGAGATCCCTGATGGAGAACTCAG |
| | | ATAGATGCCAGGACATCCGGAACCTGGCCTTTCTTGGG |
| | | ATAGCCTACAACACCTTGCTGAGGATAGCAGAAATTGC |
| | | CAGAATTCGGGTCAAGGACATTAGCAGGACAGATGGG |
| | | GGCAGGATGCTCATCCACATTGGCCGGACTAAAACCCT |
| | | TGTTTCAACTGCAGGCGTGGAAAAAGCCTTGAGCTTAG |
| | | GTGTCACCAAGCTGGTGGAGAGATGGATCAGCGTCTCC |
| | | GGAGTTGCAGACGACCCAAATAATTATCTCTTCTGTCG |
| | | TGTTCGGAAGAACGGAGTTGCAGCGCCCTCGGCTACCA |
| | | GCCAACTAAGCACGAGAGCTCTGGAGGGCATTTTTGAG |
| | | GCCACTCATCGCCTGATCTATGGAGCAAAAGATGACTC |
| | | CGGGCAGAGATACCTGGCATGGAGTGGTCATAGTGCTC |
| | | GTGTCGGTGCTGCAAGAGATATGGCCCGGGCTGGGGTT |
| | | TCCATACCTGAAATCATGCAGGCTGGTGGCTGGACAAA |
| | | CGTGAACATTGTGATGAACTACATCAGGAATCTAGATT |
| | | CTGAGACAGGAGCCATGGTGCGATTACTGGAAGATGGC |
| | | GATTGA |
| 5 | mPUMA | ATGGCCAGAGCAAGGCAAGAAGGCAGCAGTCCAGAAC |
| | | CTGTGGAAGGATTGGCCAGGGACTCCCCGAGACCATTT |
| | | CCTCTCGGCCGGCTCATGCCCAGTGCTGTCAGCTGCTCA |
| | | TTGTGTGAGCCAGGCCTTCCAGCAGCACCAGCTGCACC |
| | | CGCCCTGTTGCCAGCAGCTTACCTGTGCGCCCCTACTGC |
| | | CCCCCCTGCCGTGACCGCGGCACTTGGTGGACCCCGCT |
| | | GGCCTGGTGGGCACAGAAGCAGACCGCGAGGCCCCAG |
| | | ACCAGATGGCCCCCAGCCTTCCCTGTCTCCCGCGCAGC |
| | | AGCACCTGGAGTCCCCTGTACCATCAGCCCCGGAAGCT |
| | | CTGGCAGGGGGTCCTACACAAGCCGCTCCTGGGGTTCG |
| | | GGTGGAGGAAGAAGAGTGGGCCCGAGAGATCGGGGCT |
| | | CAGCTGCGTAGGATGGCTGATGACCTCAATGCTCAGTA |
| | | TGAACGCCGGCGTCAGGAGGAGCAGCATAGGCATCGC |
| | | CCCTCTCCCTGGAGGGTCATGTACAACCTATTCATGGG |
| | | ATTACTGCCTCTCCCTCGGGACCCAGGAGCCCCAGAGA |
| | | TGGAGCCCAACTGA |
| 6 | mBAK1 | ATGGCCTCCGGCCAGGGCCCCGGCCCCCCCAAGGTGGG |
| | | CTGCGACGAGTCCCCCTCCCCCTCCGAGCAGCAGGTGG |
| | | CCCAGGACACCGAGGAGGTGTTCCGCTCCTACGTGTTC |
| | | TACCTGCACCAGCAGGAGCAGGAGACCCAGGGCGCCG |
| | | CCGCCCCCGCCAACCCCGAGATGGACAACCTGCCCCTG |
| | | GAGCCCAACTCCATCCTGGGCCAGGTGGGCCGCCAGCT |
| | | GGCCCTGATCGGCGACGACATCAACCGCCGCTACGACA |
| | | CCGAGTTCCAGAACCTGCTGGAGCAGCTGCAGCCCACC |
| | | GCCGGCAACGCCTACGAGCTGTTCACCAAGATCGCCTC |
| | | CTCCCTGTTCAAGTCCGGCATCTCCTGGGGCCGCGTGGT |
| | | GGCCCTGCTGGGCTTCGGCTACCGCCTGGCCCTGTACG |
| | | TGTACCAGCGCGGCCTGACCGGCTTCCTGGGCCAGGTG |
| | | ACCTGCTTCCTGGCCGACATCATCCTGCACCACTACATC |
| | | GCCCGCTGGATCGCCCAGCGCGGCGGCTGGGTGGCCGC |
| | | CCTGAACTTCCGCCGCGACCCCATCCTGACCGTGATGG |
| | | TGATCTTCGGCGTGGTGCTGCTGGGCCAGTTCGTGGTG |
| | | CACCGCTTCTTCCGCTCCTAG |
| 7 | mCASP3 | ATGGAGAACAACAAGACCTCCGTGGACTCCAAGTCCAT |
| | | CAACAACTTCGAGGTGAAGACCATCCACGGCTCCAAGT |
| | | CCGTGGACTCCGGCATCTACCTGGACTCCTCCTACAAG |
| | | ATGGACTACCCCGAGATGGGCATCTGCATCATCATCAA |
| | | CAACAAGAACTTCCACAAGTCCACCGGCATGTCCTCCC |
| | | GCTCCGGCACCGACGTGGACGCCGCCAACCTGCGCGAG |
| | | ACCTTCATGGGCCTGAAGTACCAGGTGCGCAACAAGAA |
| | | CGACCTGACCCGCGAGGACATCCTGGAGCTGATGGACT |
| | | CCGTGTCCAAGGAGGACCACTCCAAGCGGTCCTCCTTC |
| | | GTGTGCGTGATCCTGTCCCACGGCGACGAGGGCGTGAT |
| | | CTACGGCACCAACGGCCCCGTGGAGCTGAAGAAGCTG |
| | | ACCTCCTTCTTCCGCGGCGACTACTGCCGCTCCCTGACC |
| | | GGCAAGCCCAAGCTGTTCATCATCCAGGCCTGCCGCGG |
| | | CACCGAGCTGGACTGCGGCATCGAGACCGACTCCGGCA |
| | | CCGACGAGGAGATGGCCTGCCAGAAGATCCCCGTGGA |
| | | GGCCGACTTCCTGTACGCCTACTCCACCGCCCCCGGCT |
| | | ACTACTCCTGGCGCAACTCCAAGGACGGCTCCTGGTTC |
| | | ATCCAGTCCCTGTGCTCCATGCTGAAGCTGTACGCCCA |
| | | CAAGCTGGAGTTCATGCACATCCTGACCCGCGTGAACC |
| | | GCAAGGTGGCCACCGAGTTCGAGTCCTTCTCCCTGGAC |
| | | TCCACCTTCCACGCCAAGAAGCAGATCCCCTGCATCGT |
| | | GTCCATGCTGACCAAGGAGCTGTACTTCTACCACTAG |
| | | ATGACCGACGACCAGGACTGCGCCGCCGAGCTGGAGA |
| 8 | mCASP7 | AGGTGGACTCCTCCTCCGAGGACGGCGTGGACGCCAAG |
| | | CCCGACCGCTCCTCCATCATCTCCTCCATCCTGCTGAAG |
| | | AAGAAGCGCAACGCCTCCGCCGGCCCCGTGCGCACCGG |
| | | CCGCGACCGCGTGCCCACCTACCTGTACCGCATGGACT |
| | | TCCAGAAGATGGGCAAGTGCATCATCATCAACAACAAG |
| | | AACTTCGACAAGGCCACCGGCATGGACGTGCGCAACG |
| | | GCACCGACAAGGACGCCGGCGCCCTGTTCAAGTGCTTC |
| | | CAGAACCTGGGCTTCGAGGTGACCGTGCACAACGACTG |
| | | CTCCTGCGCCAAGATGCAGGACCTGCTGCGCAAGGCCT |
| | | CCGAGGAGGACCACTCCAACTCCGCCTGCTTCGCCTGC |
| | | GTGCTGCTGTCCCACGGCGAGGAGGACCTGATCTACGG |
| | | CAAGGACGGCGTGACCCCCATCAAGGACCTGACCGCCC |
| | | ACTTCCGCGGCGACCGCTGCAAGACCCTGCTGGAGAAG |
| | | CCCAAGCTGTTCTTCATCCAGGCCTGCCGCGGCACCGA |
| | | GCTGGACGACGGCATCCAGGCCGACTCCGGCCCCATCA |
| | | ACGACATCGACGCCAACCCCCGCAACAAGATCCCCGTG |
| | | GAGGCCGACTTCCTGTTCGCCTACTCCACCGTGCCCGG |
| | | CTACTACTCCTGGCGCAACCCCGGCAAGGGCTCCTGGT |
| | | TCGTGCAGGCCCTGTGCTCCATCCTGAACGAGCACGGC |
| | | AAGGACCTGGAGATCATGCAGATCCTGACCCGCGTGAA |
| | | CGACCGCGTGGCCCGCCACTTCGAGTCCCAGTCCGACG |
| | | ACCCCCGCTTCAACGAGAAGAAGCAGATCCCCTGCATG |
| | | GTGTCCATGCTGACCAAGGAGCTGTACTTCTCCCGCTA |
| | | G |
| 9 | miRts | TGATAATAGCAAACACCATTGTCACACTCCAGCTGGAG |
| | | CCTCGGTGGCCATGCTTCTTGCCCCTTGGGCCCAAACAC |
| | | CATTGTCACACTCCATCCCCCCAGCCCCTCCTCCCCTTC |
| | | CTGCACCCGTACCCCCCAAACACCATTGTCACACTCCA |
| | | GTGGTCTTTGAATAAAGTCTGAGTGGGCGGC |
| 10 | ABE8e- | ATGAAACGGACAGCCGACGGAAGCGAGTTCGAGTCAC |
| | NRCH | CAAAGAAGAAGCGGAAAGTCTCTGAGGTGGAGTTTTCC |
| | | CACGAGTACTGGATGAGACATGCCCTGACCCTGGCCAA |
| | | GAGGGCACGGGATGAGAGGGAGGTGCCTGTGGGAGCC |
| | | GTGCTGGTGCTGAACAATAGAGTGATCGGCGAGGGCTG |
| | | GAACAGAGCCATCGGCCTGCACGACCCAACAGCCCATG |
| | | CCGAAATTATGGCCCTGAGACAGGGCGGCCTGGTCATG |
| | | CAGAACTACAGACTGATTGACGCCACCCTGTACGTGAC |
| | | ATTCGAGCCTTGCGTGATGTGCGCCGGCGCCATGATCC |
| | | ACTCTAGGATCGGCCGCGTGGTGTTTGGCGTGAGGAAC |
| | | TCAAAAAGAGGCGCCGCAGGCTCCCTGATGAACGTGCT |
| | | GAACTACCCCGGCATGAATCACCGCGTCGAAATTACCG |
| | | AGGGAATCCTGGCAGATGAATGTGCCGCCCTGCTGTGC |
| | | GATTTCTATCGGATGCCTAGACAGGTGTTCAATGCTCA |
| | | GAAGAAGGCCCAGAGCTCCATCAACTCCGGAGGATCTA |
| | | GCGGAGGCTCCTCTGGCTCTGAGACACCTGGCACAAGC |
| | | GAGAGCGCAACACCTGAAAGCAGCGGGGGCAGCAGCG |
| | | GGGGGTCAGACAAGAAGTACAGCATCGGCCTGACCAT |
| | | CGGCACCAACTCTGTGGGCTGGGCCGTGATCACCGACG |
| | | AGTACAAGGTGCCCAGCAAGAAATTCAAGGTGCTGGG |
| | | CAACACCGACCGGCACAGCATCAAGAAGAACCTGATC |
| | | GGAGCCCTGCTGTTCGACAGCGGCGAAACAGCCGAGG |
| | | CCACCCGGCTGAAGAGAACCGCCAGAAGAAGATACAC |
| | | CAGACGGAAGAACCGGATCTGCTATCTGCAAGAGATCT |
| | | TCAGCAACGAGATGGCCAAGGTGGACGACAGCTTCTTC |
| | | CACAGACTGGAAGAGTCCTTCCTGGTGGAAGAGGATAA |
| | | GAAGCACGAGCGGCACCCCATCTTCGGCAACATCGTGG |
| | | ACGAGGTGGCCTACCACGAGAAGTACCCCACCATCTAC |
| | | CACCTGAGAAAGAAACTGGTGGACAGCACCGACAAGG |
| | | CCGACCTGCGGCTGATCTATCTGGCCCTGGCCCACATG |
| | | ATCAAGTTCCGGGGCCACTTCCTGATCGAGGGCGACCT |
| | | GAACCCCGACAACAGCGACGTGGACAAGCTGTTCATCC |
| | | AGCTGGTGCAGACCTACAACCAGCTGTTCGAGGAAAAC |
| | | CCCATCAACGCCAGCGGCGTGGACGCCAAGGCCATCCT |
| | | GTCTGCCAGACTGAGCAAGAGCAGACGGCTGGAAAAT |
| | | CTGATCGCCCAGCTGCCCGGCGAGAAGAAGAATGGCCT |
| | | GTTCGGAAACCTGATTGCCCTGAGCCTGGGCCTGACCC |
| | | CCAACTTCAAGAGCAACTTCGACCTGGCCGAGGATGCC |
| | | AAACTGCAGCTGAGCAAGGACACCTACGACGACGACC |
| | | TGGACAACCTGCTGGCCCAGATCGGCGACCAGTACGCC |
| | | GACCTGTTTCTGGCCGCCAAGAACCTGTCCGACGCCAT |
| | | CCTGCTGAGCGACATCCTGAGAGTGAACACCGAGATCA |
| | | CCAAGGCCCCCCTGAGCGCCTCTATGGTGAAGAGATAC |
| | | GACGAGCACCACCAGGACCTGACCCTGCTGAAAGCTCT |
| | | CGTGCGGCAGCAGCTGCCTGAGAAGTACAAAGAGATTT |
| | | TCTTCGACCAGAGCAAGAACGGCTACGCCGGCTACATT |
| | | GACGGCGGAGCCAGCCAGGAAGAGTTCTACAAGTTCAT |
| | | CAAGCCCATCCTGGAAAAGATGGACGGCACCGAGGAA |
| | | CTGCTCGTGAAGCTGAACAGAGAGGACCTGCTGCGGAA |
| | | GCAGCGGACCTTCGACAACGGCATTATCCCCCACCAGA |
| | | TCCACCTGGGAGAGCTGCACGCCATTCTGCGGCGGCAG |
| | | GGCGATTTTTACCCATTCCTGAAGGACAACCGGGAAAA |
| | | GATCGAGAAGATCCTGACCTTCCGCATCCCCTACTACG |
| | | TGGGCCCTCTGGCCAGGGGAAACAGCAGATTCGCCTGG |
| | | ATGACCAGAAAGAGCGAGGAAACCATCACCCCCTGGA |
| | | ACTTCGAGGAAGTGGTGGACAAGGGCGCTTCCGCCCAG |
| | | AGCTTCATCGAGCGGATGACCAACTTCGATAAGAACCT |
| | | GCCCAACGAGAAGGTGCTGCCCAAGCACAGCCTGCTGT |
| | | ACGAGTACTTCACCGTGTATAACGAGCTGACCAAAGTG |
| | | AAATACGTGACCGAGGGAATGAGAAAGCCCGCCTTCCT |
| | | GAGCGGCGAGCAGAAAAAGGCCATCGTGGACCTGCTG |
| | | TTCAAGACCAACCGGAAAGTGACCGTGAAGCAGCTGA |
| | | AAGAGGACTACTTCAAGAAAATCGAGTGCTTCGACTCC |
| | | GTGGAAATCTCCGGCGTGGAAGATCGGTTCAACGCCTC |
| | | CCTGGGCACATACCACGATCTGCTGAAAATTATCAAGG |
| | | ACAAGGACTTCCTGGACAATGAGGAAAACGAGGACAT |
| | | TCTGGAAGATATCGTGCTGACCCTGACACTGTTTGAGG |
| | | ACAGAGAGATGATCGAGGAACGGCTGAAAACCTATGC |
| | | CCACCTGTTCGACGACAAAGTGATGAAGCAGCTGAAGC |
| | | GGCTGAGATACACCGGCTGGGGCAGGCTGAGCCGGAA |
| | | GCTGATCAACGGCATCCGGGACAAGCAGTCCGGCAAG |
| | | ACAATCCTGGATTTCCTGAAGTCCGACGGCTTCGCCAA |
| | | CAGAAACTTCATGCAGCTGATCCACGACGACAGCCTGA |
| | | CCTTTAAAGAGGACATCCAGAAAGCCCAGGTGTCCGGC |
| | | CAGGGCGATAGCCTGCACGAGCACATTGCCAATCTGGC |
| | | CGGCAGCCCCGCCATTAAGAAGGGCATCCTGCAGACAG |
| | | TGAAGGTGGTGGACGAGCTCGTGAAAGTGATGGGCGG |
| | | CCACAAGCCCGAGAACATCGTGATCGAAATGGCCAGA |
| | | GAGAACCAGACCACCCAGAAGGGACAGAAGAACAGCC |
| | | GCGAGAGAATGAAGCGGATCGAAGAGGGCATCAAAGA |
| | | GCTGGGCAGCCAGATCCTGAAAGAACACCCCGTGGAA |
| | | AACACCCAGCTGCAGAACGAGAAGCTGTACCTGTACTA |
| | | CCTGCAGAATGGGCGGGATATGTACGTGGACCAGGAA |
| | | CTGGACATCAACCGGCTGTCCGACTACGATGTGGACCA |
| | | TATCGTGCCTCAGAGCTTTCTGAAGGACGACTCCATCG |
| | | ACAACAAGGTGCTGACCAGAAGCGACAAGAACCGGGG |
| | | CAAGAGCGACAACGTGCCCTCCGAAGAGGTCGTGAAG |
| | | AAGATGAAGAACTACTGGCGGCAGCTGCTGAACGCCA |
| | | AGCTGATTACCCAGAGAAAGTTCGACAATCTGACCAAG |
| | | GCCGAGAGAGGCGGCCTGAGCGAACTGGATAAGGCCG |
| | | GCTTCATCAAGAGACAGCTGGTGGAAACCCGGCAGATC |
| | | ACAAAGCACGTGGCACAGATCCTGGACTCCCGGATGAA |
| | | CACTAAGTACGACGAGAATGACAAGCTGATCCGGGAA |
| | | GTGAAAGTGATCACCCTGAAGTCCAAGCTGGTGTCCGA |
| | | TTTCCGGAAGGATTTCCAGTTTTACAAAGTGCGCGAGA |
| | | TCAACAACTACCACCACGCCCACGACGCCTACCTGAAC |
| | | GCCGTCGTGGGAACCGCCCTGATCAAAAAGTACCCTAA |
| | | GCTGGAAAGCGAGTTCGTGTACGGCGACTACAAGGTGT |
| | | ACGACGTGCGGAAGATGATCGCCAAGAGCGAGCAGGA |
| | | AATCGGCAAGGCTACCGCCAAGTACTTCTTCTACAGCA |
| | | ACATCATGAACTTTTTCAAGACCGAGATTACCCTGGCC |
| | | AACGGCGAGATCCGGAAGCGGCCTCTGATCGAGACAA |
| | | ACGGCGAAACCGGGGAGATCGTGTGGGATAAGGGCCG |
| | | GGATTTTGCCACCGTGCGGAAAGTGCTGAGCATGCCC |
| | | CAAGTGAATATCGTGAAAAAGACCGAGGTGCAGACAG |
| | | GCGGCTTCAGCAAAGAGTCTATCCTGCCCAAGGGCAAC |
| | | AGCGATAAGCTGATCGCCAGAAAGAAGGACTGGGACC |
| | | CTAAGAAGTACGGCGGCTTCAACAGCCCCACCGTGGCC |
| | | TATTCTGTGCTGGTGGTGGCCAAAGTGGAAAAGGGCAA |
| | | GTCCAAGAAACTGAAGAGTGTGAAAGAGCTGCTGGGG |
| | | ATCACCATCATGGAAAGAAGCAGCTTCGAGAAGAATCC |
| | | CATCGACTTTCTGGAAGCCAAGGGCTACAAAGAAGTGA |
| | | AAAAGGACCTGATCATCAAGCTGCCTAAGTACTCCCTG |
| | | TTCGAGCTGGAAAACGGCCGGAAGAGAATGCTGGCCTC |
| | | TGCCGGCGTGCTGCAGAAGGGAAACGAACTGGCCCTGC |
| | | CCTCCAAATATGTGAACTTCCTGTACCTGGCCAGCCACT |
| | | ATGAGAAGCTGAAGGGCTCCCCCGAGGATAATGAGCA |
| | | GAAACAGCTGTTTGTGGAACAGCACAAGCACTACCTGG |
| | | ACGAGATCATCGAGCAGATCAGCGAGTTCTCCAAGAGA |
| | | GTGATCCTGGCCGACGCTAATCTGGACAAAGTGCTGTC |
| | | CGCCTACAACAAGCACCGGGATAAGCCCATCAGAGAG |
| | | CAGGCCGAGAATATCATCCACCTGTTTACCCTGACCAA |
| | | TCTGGGAGCCCCTGCCGCCTTCAAGTACTTTGACACCA |
| | | CCATCAACCGGAAGCAATACAACACGACCAAAGAGGT |
| | | GCTGGACGCCACCCTGATCCGTCAGAGCATCACCGGCC |
| | | TGTACGAGACACGGATCGACCTGTCTCAGCTGGGAGGT |
| | | GACTCTGGCGGCTCAAAAAGAACCGCCGACGGCAGCG |
| | | AATTCGAGCCCAAGAAGAAGAGGAAAGTCTAA |
| 11 | HBBG-Makassar | UUCUCCACAGGAGUCAGGUG |
| | sgRNA | |

### Example 2: Treatment Protocol for Improved Stem Cell Engraftment

The research team has developed a new preconditioning protocol to further improve the stem cell engraftment by treating animals with Benadryl and Dexamethasone before and after PUMA mRNA treatment to reduce the histamine induced toxicity and secondary inflammation. This approach allows for higher PUMA mRNA dosage which leads to more efficient pre-conditioning and better donor cell engraftment.

### Initial Protocol:

1× dose of 1 μg/mouse w/o additional treatment, 10E+06 GFP bone marrow cells injected after 6.5 days resulted in engraftment levels: 2-3% in all hematopoietic tissues, maintained in primary HSCT.

New protocols and experimental cohorts using Hbbth3/+ Thalassemia model:

Increased dosing (up to 4.5 μg PUMA particles/mouse) w/20 mg/g Benadryl 15 min before and 2 mg/Kg Dexamethasone 5 min after ^{CD117}LNP/PUMA^{miR122} pre-conditioning leads to greater donor erythroid engraftment (almost 8 times higher at 4 mo).

1× thal mouse ^{CD117}LNP/PUMA^{miR122} preconditioning with (2 μg/mouse) resulted in increasing donor RBC over time (13.3% at 3 mo) and improved erythropoiesis.

2× thal mice ^{CD117}LNP/PUMA^{miR122} preconditioning (2.5 μg/mouse) resulted in 1 died and 1 shows increasing donor RBC over time (21.2% at 3 mo) and improved erythropoiesis (↑Red Blood Cell count, Hemoglobin, Hematocrit and ↓ Reticulocyte %.

The Hbbth3/+ model lacks both adult beta globin genes in one allele and present features comparable to those of BT intermedia patients. Hb is lower than in normal mice as well as Hematocrit (HCT) and RBC counts. The reticulocyte count instead is elevated. The outcome measures in mice treated with GFP-BM after ^{CD117}LNPPUMA^{miR122} pre-conditioning includes flow analysis to determine donor chimerism (% GFP cells in peripheral blood) over time and CBC analyses to follow erythroid parameters, along with RBC morphology analyses by Giemsa staining.

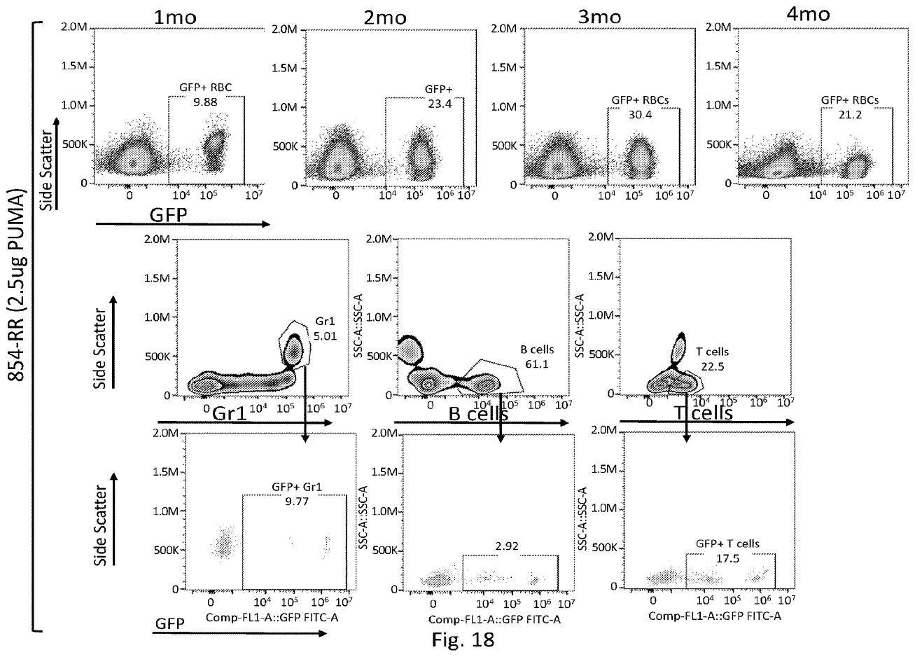

FIG. 18 shows Top: Percentage of GFP+ red blood cells (Flow analyses) in a representative mouse (854-RR) preconditioned with ^{CD117}LNP-PUMA^{miR122} at 2.5 ug/mouse, using new protocol (pre/post treatments with Benadryl/Dexamethasone), followed by injection of healthy GPB BM. Middle and Bottom: Percentage of GFP+ white blood cells, including myeloid (Gr1), and lymphoid (B and T cells) (Flow analyses) in a representative mouse (854-RR) preconditioned with ^{CD117}LNP-PUMA^{miR122} at 2.5 ug/mouse (data at 4 month post BMT), using new protocol (pre/post treatments with Benadryl/Dexamethasone).

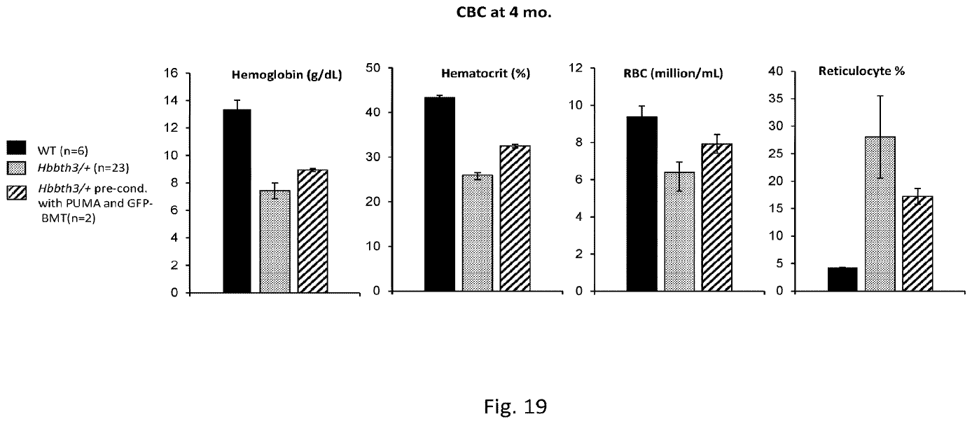

FIG. 19 shows that there is an improvement of BT features in thalassemic mice 854-RR and RL treated with GFP BM after ^{CD117}LNP-PUMA^{miR122} preconditioning: Hemoglobin, Hematocrit and RBC counts are increased, while reticulocyte % (a hallmark of BT) are reduced.

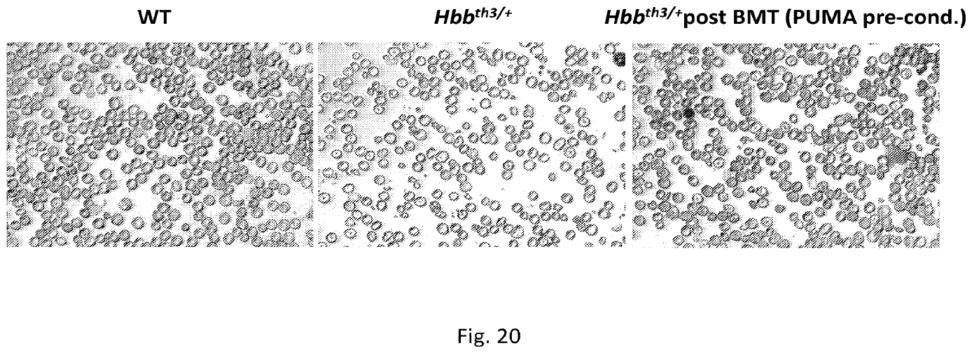

FIG. 20 shows that there is amelioration of morphology in RBC analyzed after erythroid Giemsa staining. Comparison between WT, BT and BT mice transplanted with GFP-BM after ^{CD117}LNP-PUMA^{miR122} preconditioning. In treated mouse (854RL), RBC's size, shape, color and number are improved, resembling features similar to those seeing in WT RBC

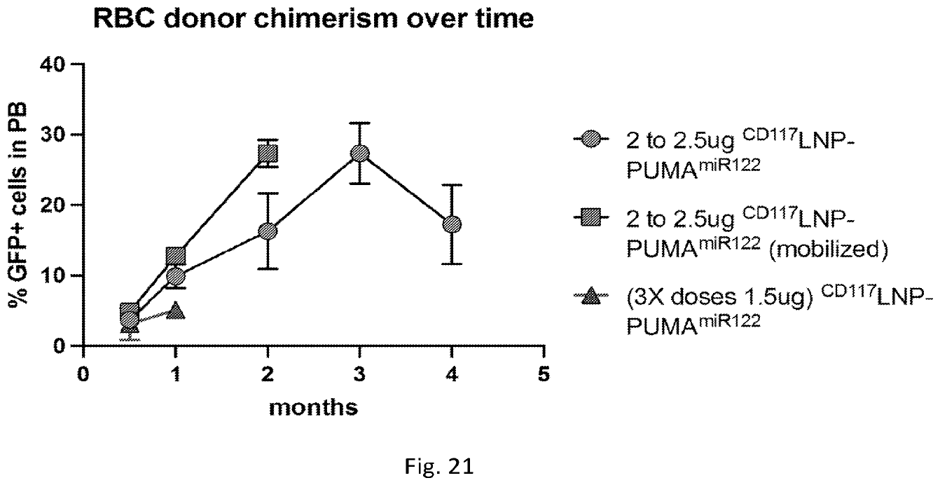

FIG. 21 shows a summary of % GFP+ red blood cells (Flow analyses) over 4 months in 3 cohorts: 1) mice preconditioned with ^{CD117}LNP-PUMA^{miR122} at 2.5 μg/mouse, using new protocol (pre/post treatments with Benadryl/Dexamethasone) (n=5, represented by solid circles, with 3 out of 5 animals up to 2 months); 2) mice preconditioned mobilized with a combination of bortezomib and plerixafor 18 hours prior to preconditioning with ^{CD117}LNP-PUMA^{miR122} at 2.5 μg/mouse, using new protocol (pre/post treatments with Benadryl/Dexamethasone) (n=2, represented by solid squares, 3 additional mice are in progress); 3) mice preconditioned with ^{CD117}LNP-PUMA^{miR122} at 3 consecutive doses of 1.5 μg/mouse, over 3 days (injected 24 hours apart) using new protocol (pre/post treatments with Benadryl/Dexamethasone) (n=3, represented by solid triangles, with 2 out of 3 animals followed up to 2 weeks), All animals have been injected with 10E+06 GFP+BM cells from donor healthy mice 6.5 days after ^{CD117}LNP-PUMA^{miR122} preconditioning.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A composition for targeted delivery of an apoptosis-inducing agent to a target hematopoietic stem cell (HSC), the composition comprising an agent and a delivery vehicle, wherein the delivery vehicle comprises a CD117 targeting moiety specific for binding to the HSC, and wherein the agent comprises an mRNA molecule encoding a pro-apoptotic protein.

2. The composition of claim 1, wherein the proapoptotic protein is p53 upregulated modulator of apoptosis (PUMA), BCL2 Antagonist/Killer 1 (Bak-1), Caspase 3 (Cas-3) or Caspase 9 (Cas-9).

3. The composition of claim 1, wherein the mRNA comprises SEQ ID NO: 5, SEQ ID NO:6, SEQ ID NO:7, SEQ ID NO:8 or a fragment or variant thereof.

4. The composition of claim 1, wherein the mRNA molecule is an isolated nucleoside-modified mRNA molecule.

5. The composition of claim 4, wherein the at least one isolated nucleoside-modified RNA comprises at least one selected from the group consisting of pseudouridine and 1-methyl-pseudouridine

6. The composition of claim 1, wherein the mRNA molecule comprises a binding site for miR-122.

7. The composition of claim 6, wherein the miR-122 binding site is in the 3′UTR of the mRNA molecule.

8. The composition of any one of claims 1-7, wherein the delivery vehicle comprises a lipid nanoparticle (LNP).

9. The composition of claim 8, wherein the mRNA is encapsulated within the LNP.

10. A method of treating a disease or disorder in a subject in need thereof, the method comprising administering a composition of any one of claims 1-9 to the subject.

11. The method of claim 10, wherein the disease or disorder is selected from the group consisting of blood monogenic disorders, genetic defects, bone marrow genetic defects, cancers, platelet disorders, red cell disorders, immunodeficiencies, non-hematologic diseases, metabolic disease and autoimmune diseases.

12. The method of claim 11, wherein the composition is administered by a delivery route selected from the group consisting of intravenous (IV), intraosseous infusion (IO), intraperitoneal (IP), intradermal, subcutaneous, inhalation, intranasal, and intramuscular.

13. A method of preconditioning a subject for hematopoietic stem cell therapy (HSCT), the method comprising administering a composition of any one of claims 1-9 to the subject.

14. The method of claim 13, wherein the HSCT is autologous HSCT.

15. The method of claim 13, wherein the composition is administered by a delivery route selected from the group consisting of intravenous (IV), intraosseous infusion (IO), intraperitoneal (IP), intradermal, subcutaneous, inhalation, intranasal, and intramuscular.

16. The method of claim 13, wherein the method further comprises administering at least one of an antihistamine agent and an anti-inflammatory agent in combination with the composition of any one of claims 1-9.

17. The method of claim 13, wherein the method comprises administering an antihistamine agent and an anti-inflammatory agent in combination with the composition of any one of claims 1-9.

18. The method of claim 13, wherein the antihistamine agent is administered 1 day to 1 minute prior to administration of the composition of any one of claims 1-9, and the anti-inflammatory agent is administered 1 minute to 1 day following administration of the composition of any one of claims 1-9.

19. A method of depleting HSCs in a subject in need thereof, the method comprising administering a composition of any one of claims 1-9 to the subject.

20. The method of claim 19, wherein the subject has a disease or disorder selected from the group consisting of blood monogenic disorders, genetic defects, bone marrow genetic defects, cancers, platelet disorders, red cell disorders, immunodeficiencies, non-hematologic diseases, metabolic disease and autoimmune diseases.

21. The method of claim 19, wherein the composition is administered by a delivery route selected from the group consisting of intravenous (IV), intraosseous infusion (IO), intraperitoneal (IP), intradermal, subcutaneous, inhalation, intranasal, and intramuscular.

22. The method of claim 19, wherein the method further comprises administering at least one of an antihistamine agent and an anti-inflammatory agent in combination with the composition of any one of claims 1-9.

23. The method of claim 19, wherein the method comprises administering an antihistamine agent and an anti-inflammatory agent in combination with the composition of any one of claims 1-9.

24. The method of claim 23, wherein the antihistamine agent is administered 1 day to 1 minute prior to administration of the composition of any one of claims 1-9, and the anti-inflammatory agent is administered 1 minute to 1 day following administration of the composition of any one of claims 1-9.
